(19) United States
(12) Patent Application Publication
(10) Pub. No.: US 2023/0270718 A1
(43) Pub. Date: Aug. 31, 2023

(54) METHODS OF TREATING DIABETIC KIDNEY DISEASE

(71) Applicants: Chinook Therapeutics, Inc., Seattle, WA (US); AbbVie Inc., North Chicago, IL (US)

(72) Inventors: Joel Z. Melnick, North Chicago, IL (US); Michael G. Miller, North Chicago, IL (US); Tingting YI, North Chicago, IL (US); Hiddo Lambers Heerspink, North Chicago, IL (US); Andrew James King, Seattle, WA (US); Sarah B. Noonberg, Seattle, WA (US)

(21) Appl. No.: 17/995,798

(22) PCT Filed: Apr. 12, 2021
(86) PCT No.: PCT/US2021/026803

§ 371 Date: Oct. 7, 2022

Related U.S. Application Data
(60) Provisional application No. 63/119,806, filed on Dec. 1, 2020.
(60) Provisional application No. 63/008,099, filed on Apr. 10, 2020.

(51) Int. Cl. A61K 31/4025; A61K 45/06; A61K 31/7042; A61K 31/7048; A61K 31/70; A61K 31/382; A61P 3/10; A61P 13/12

(52) U.S. Cl. CPC A61K 31/4025; A61K 45/06; A61K 31/7042; A61K 31/7048; A61K 31/70; A61K 31/382; A61P 3/10; A61P 13/12

(57) ABSTRACT

Provided herein are methods of treating diabetic kidney disease comprising administering a therapeutically effective amount of atrasentan, or a pharmaceutically acceptable salt thereof, and a SGLT-2 inhibitor to a subject in need thereof.

### CROSS-REFERENCE TO RELATED APPLICATIONS

This application is a U.S. National Stage Entry of International Patent Application No. PCT/US2021/026803, filed on Apr. 12, 2021, which claims priority to U.S. Provisional Patent Application No. 63/008,099, filed on Apr. 10, 2020; and U.S. Provisional Patent Application No. 63/119,806, filed Dec. 1, 2020, both of which are herein incorporated by reference in their entireties.

### BACKGROUND

Diabetic kidney disease (DKD), also referred to as diabetic nephropathy, is a kidney disease associated with diabetes, and is one of the leading causes of kidney disease. DKD is considered a secondary glomerular disease, where kidney disease develops secondarily to an identified systemic cause, in the case of DKD as a microvascular complication to long-standing diabetes. See, e.g. Dattani and McAdoo, Medicine, 47(10), pp. 644-648 (2019). The pathogenesis of DKD involves chronically elevated blood glucose levels that can cause glucose toxicity to renal cells, especially kidney endothelial cells, and systemic and renal hemodynamic factors associated with hypertension that result in shear stress being transmitted to resident glomerular cells are the key pathogenic drivers of DKD. See, e.g. Thomas et al., Nat. Rev. Disease Primers. 1, pp. 15018-15026 (2015). Multiple factors dysregulated in diabetic subjects, including metabolic components such as hyperglycemia, dyslipidemia and oxidative stress, and hemodynamic factors such as vasoactive substances associated with hypertension, all stimulate renal endothelin-1 (ET-1) formation. In addition, DKD is frequently observed in the elderly population due to a history of diabetes prior to manifestation of DKD, and aging is also associated with increased ET-1 production in the kidney, which is a risk factor for developing DKD. See, e.g. Kohan, Kidney Int., 86(5), pp. 896-904 (2014).

DKD patients present with a range of symptoms, such as microalbuminuria in the early stages, albuminuria or macroalbuminuria in the later stages, and finally end stage renal disease (ESRD). Patients with DKD often exhibit significantly increased expression of endothelin 1 (ET-1) and endothelin-1 receptor type A (ET-RA) in the kidney. Increased expression of endothelins positively correlates with albuminuria, one of the hallmark indicators of DKD.

Current treatments for DKD are directed to managing glucose levels and managing blood pressure, typically with diuretics, angiotensin-converting enzyme (ACE) inhibitors, and angiotensin II receptor blockers (ARBs). Treatment with ACE inhibitors and ARBs, alone or in combination with diuretics, is not curative, however, and often fails to provide complete disease management. As a result, endothelin A receptor antagonists have been investigated as an additional treatment option for patients with DKD. One selective endothelin A receptor antagonist, atrasentan, when administered in combination with ACE inhibitors and ARBs has been shown to provide an additional option for disease management (by reducing albuminuria without increased sodium retention). Heerspink et al., Lancet 393:1937-1947 (2019) (published online Apr. 14, 2019). Atrasentan is a selective endothelin A (ETA) receptor antagonist (ETA Ki˜34 pM; ETB Ki˜63 nM, ETA selectivity ˜1800×). See, e.g. Wu-Wong et al., Clin. Sci. (Lond.), 103(48), pp. 107s-111s (2002). Selective ETA receptor antagonists block ETA function, while minimally effecting the ETB receptor, providing beneficial renal effects including vasodilation and reduction of inflammation, while still enabling ET-1 clearance. See e.g., Jandeleit-Dahm and Watson, Curr. Opin. Nephrol. Hypertens., 21(1), pp. 66-71 (2012); see also, Nakamura, et al., Nephron, Vol. 72, pp. 454-460 (1996). While ETA receptor antagonists increase sodium and water retention by the kidney, this is typically clinically manageable. See, e.g., Saleh, et al., J. Pharm. Exp. Ther., 338(1), pp. 263-270 (2011).

In the SONAR study, patients with type 2 diabetes receiving a low dose of atrasentan in combination with an ACE inhibitor or ARB and a diuretic exhibited a significant reduction in the risk of doubling of serum creatinine (indicative of kidney disease progression) or ESRD. See, e.g., Heerspink, et al., The Lancet, 393, pp 1937-1947 (2019). Some patients receiving the combination, however, exhibited fluid retention or an increase in B-type natriuretic peptide (BNP), as compared to the placebo group. Heerspink (2019). Thus, DKD patients may benefit from further treatment options, in addition to therapy with combinations of ACE inhibitors and ARBs, diuretics, and atrasentan.

### SUMMARY

The present application provides methods of treating diabetic kidney disease (DKD) in a subject in need thereof by administering a therapeutically effective amount of atrasentan, or a pharmaceutically acceptable salt thereof, and a therapeutically effective amount of a SGLT-2 inhibitor to a subject in need thereof. The present application is based, in part, on the unexpected observation that the combination of atrasentan (or a pharmaceutically acceptable salt thereof) and a SGLT-2 inhibitor (or a pharmaceutically acceptable salt thereof) provided an additional benefit to patients by further reducing the urine albumin to creatinine ratio (UACR) of the subject, as compared to treatment with atrasentan (or a pharmaceutically acceptable salt thereof) alone. The present application is also based, in part, on the unexpected observation that the combination of atrasentan (or a pharmaceutically acceptable salt thereof) and a SGLT-2 inhibitor (or a pharmaceutically acceptable salt thereof) provided an additional benefit to patients by reducing fluid retention (weight gain) by the subject, as compared to treatment with atrasentan (or a pharmaceutically acceptable salt thereof) alone.

Some embodiments provide a method of treating diabetic kidney disease (DKD), comprising administering a therapeutically effective amount of atrasentan, or a pharmaceutically acceptable salt thereof, and a therapeutically effective amount of a SGLT-2 inhibitor to a subject in need thereof.

Some embodiments provide a method of delaying progressive renal function decline in a subject having DKD, comprising administering a therapeutically effective amount of atrasentan, or a pharmaceutically acceptable salt thereof, and a therapeutically effective amount of a SGLT-2 inhibitor to the subject.

Some embodiments provide a method of treating chronic kidney disease associated with diabetes, comprising administering a therapeutically effective amount of atrasentan, or a pharmaceutically acceptable salt thereof, and a therapeutically effective amount of a SGLT-2 inhibitor to a subject in need thereof.

Some embodiments provide a method of improving treatment outcome of a subject having diabetic kidney disease, comprising administering a therapeutically effective amount of atrasentan, or a pharmaceutically acceptable salt thereof, and a therapeutically effective amount of a SGLT-2 inhibitor to a subject in need thereof.

Some embodiments provide a method of reducing the UACR of a subject having diabetic kidney disease in a subject, comprising administering a therapeutically effective amount of atrasentan, or a pharmaceutically acceptable salt thereof, and a therapeutically effective amount of a SGLT-2 inhibitor to the subject; wherein the UACR after administration of the atrasentan, or a pharmaceutically acceptable salt thereof, and the SGLT-2 inhibitor is less than the UACR prior to administration of the atrasentan, or a pharmaceutically acceptable salt thereof, and the SGLT-2 inhibitor.

Some embodiments provide a method of reducing fluid retention in a subject having diabetic kidney disease, comprising administering a therapeutically effective amount of atrasentan, or a pharmaceutically acceptable salt thereof, and a therapeutically effective amount of a SGLT-2 inhibitor to the subject; wherein fluid retention after administration of the atrasentan, or a pharmaceutically acceptable salt thereof, and the SGLT-2 inhibitor is less than fluid retention or body weight prior to administration of the atrasentan, or a pharmaceutically acceptable salt thereof, and the SGLT-2 inhibitor.

Some embodiments provide a method of reducing fluid retention in a subject having diabetic kidney disease during treatment with atrasentan, or a pharmaceutically acceptable salt thereof, comprising administering a therapeutically effective amount of a SGLT-2 inhibitor, to a subject in need thereof; wherein fluid retention after administration of the SGLT-2 inhibitor is less than fluid retention prior to administration of the SGLT-2 inhibitor.

Some embodiments provide a method of reducing a BNP level in a subject having diabetic kidney disease in a subject, comprising administering a therapeutically effective amount of atrasentan, or a pharmaceutically acceptable salt thereof, and a therapeutically effective amount of a SGLT-2 inhibitor to the subject; wherein the BNP level after administration of the atrasentan, or a pharmaceutically acceptable salt thereof, and the SGLT-2 inhibitor is less than the BNP level prior to administration of the atrasentan, or a pharmaceutically acceptable salt thereof, and the SGLT-2 inhibitor.

Some embodiments provide a method of reducing the rate of decrease of eGFR of a subject having diabetic kidney disease in a subject, comprising administering a therapeutically effective amount of atrasentan, or a pharmaceutically acceptable salt thereof, and a therapeutically effective amount of a SGLT-2 inhibitor to the subject; wherein the rate of decrease of eGFR after administration of the atrasentan, or a pharmaceutically acceptable salt thereof, and the SGLT-2 inhibitor is less than the rate of decrease of eGFR prior to administration of the atrasentan, or a pharmaceutically acceptable salt thereof, and the SGLT-2 inhibitor.

Some embodiments provide a method of stabilizing estimated glomerular filtration rate (eGFR) of a subject having diabetic kidney disease, comprising administering a therapeutically effective amount of atrasentan, or a pharmaceutically acceptable salt thereof, and a therapeutically effective amount of a SGLT-2 inhibitor to subject in need thereof wherein the eGFR of the subject following administration of the atrasentan, or a pharmaceutically acceptable salt thereof, and the SGLT-2 inhibitor is stabilized eGFR relative to a subject not administered the atrasentan, or a pharmaceutically acceptable salt thereof, and the SGLT-2 inhibitor.

Some embodiments provide a method of improving treatment outcome in a subject having diabetic kidney disease, comprising administering a synergistically effective amount of atrasentan, or a pharmaceutically acceptable salt thereof, and a synergistically effective amount of a SGLT-2 inhibitor to a subject in need thereof.

### BRIEF DESCRIPTION OF THE FIGURES

FIG. 1 illustrates the change in body weight and urinary albumin:creatinine ratio in subjects treated for six weeks with atrasentan and an SGLT-2 inhibitor versus subjects treated with atrasentan alone.

### DETAILED DESCRIPTION

### A. Definitions

In order that the present disclosure can be more readily understood, certain terms are first defined. As used in this application, except as otherwise expressly provided herein, each of the following terms shall have the meaning set forth below. Additional definitions are set forth throughout the application.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary Of Biochemistry And Molecular Biology, Revised, 2000, Oxford University Press, provide one of skill with a general dictionary of many of the terms used in this disclosure. For purposes of the present disclosure, the following terms are defined.

Units, prefixes, and symbols are denoted in their Systeme International de Unites (SI) accepted form. Numeric ranges are inclusive of the numbers defining the range. The headings provided herein are not limitations of the various aspects of the disclosure, which can be had by reference to the specification as a whole. Accordingly, the terms defined immediately below are more fully defined by reference to the specification in its entirety.

The terms “a,” “an,” or “the” as used herein not only include aspects with one member, but also include aspects with more than one member. For instance, the singular forms “a,” “an,” and “the” include plural referents unless the context clearly dictates otherwise. Thus, for example, reference to “an agent” or the like includes reference to one or more agents, and so forth.

The term “or” as used herein should in general be construed non-exclusively. For example, a claim to “a composition comprising A or B” would typically present an aspect with a composition comprising both A and B. “Or” should, however, be construed to exclude those aspects presented that cannot be combined without contradiction (e.g., a composition pH that is between 9 and 10 or between 7 and 8).

The phrase group “A or B” is typically equivalent to the group “selected from the group consisting of A and B”.

The term “and/or” where used herein is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

The terms “about” and “approximately” as used herein shall generally mean an acceptable degree of minor variation for the quantity specified given the nature or precision of the measurements. Typical, exemplary degrees of variation are within 20 percent (%), within 10%, or within 5% of a given value or range of values. Any reference to “about X” specifically indicates at least the values X, 0.95X, 0.96X, 0.97X, 0.98X, 0.99X, 1.01X, 1.02X, 1.03X, 1.04X, and 1.05X. Thus, “about X” is intended to provide written description support for a claim limitation of, e.g., “0.98X.” The terms “about” and “approximately,” particularly in reference to a given quantity, encompass and describe the given quantity itself.

When “about” is applied to the beginning of a numerical range, it applies to both ends of the range. Thus, “from about 5 to 20%” is equivalent to “from about 5% to about 20%.” When “about” is applied to the first value of a set of values, it applies to all values in that set. Thus, “about 0.5, 0.75, or 1.0 mg” is equivalent to “about 0.5, about 0.75, or about 1.0 mg.”

As used herein, the term “about”, when preceding a series of peak positions for X-ray powder diffraction (e.g., 20 values), means that all of the peaks of the group which it precedes are reported in terms of angular positions with a variability of ±0.1°. Accordingly, for example, the phrase about 8.3°, 9.7°, 10.0°, 13.0°, 15.6°, 17.2° or 19.5° means 8.3°±0.1° 9.7°±0.1° 10.0°±0.1° 13.0°±0.1° 15.6°±0.1° 17.2°±0.1° or 19.5°+0.1°.

“Treatment” or “therapy” of a subject refers to any type of intervention or process performed on, or the administration of an active agent to, the subject with the objective of reversing, alleviating, ameliorating, inhibiting, or slowing down, the onset, progression, development, severity, or recurrence of a symptom, complication, condition, or biochemical indicia associated with a disease.

“Administering” or “administration” refer to the physical introduction of a therapeutic agent to a subject, using any of the various methods and delivery systems known to those skilled in the art. Routes of administration can include oral, intravenous, intramuscular, subcutaneous, intraperitoneal, spinal or other parenteral routes of administration, for example by injection or infusion (e.g., intravenous infusion). Administration can also be performed, for example, once, a plurality of times, and/or over one or more extended periods.

Administration of two or more agents and/or compounds in combination can also be referred to herein as “co-administration.”

The terms “prophylactic” or “prophylactically” refer to any type of intervention or process performed on, or the administration of an active agent to, the subject with the objective of protecting or preventing a disease or condition from developing or at least not developing fully (e.g., to reduce the symptoms or severity of the disease or condition) such as in the development of a side effect.

A “subject” includes any human or non-human animal. The term “non-human animal” includes, but is not limited to, vertebrates such as non-human primates, sheep, dogs, and rodents such as mice, rats, and guinea pigs. In some embodiments, the subject is a human. The terms “subject” and “patient” and “individual” are used interchangeably herein.

An “effective amount” or “therapeutically effective amount” or “therapeutically effective dosage” of a drug or therapeutic agent is any amount of the drug that, when used alone or in combination with another therapeutic agent, slows down the onset of a disease or promotes disease regression as evidenced by a decrease in severity of disease symptoms, an increase in frequency and duration of disease symptom-free periods, and/or a ameliorating an impairment or disability due to the disease affliction. For example, when two or more compounds are administered at sub-therapeutic doses when administered alone, when administered in combination, this same amount may constitute a therapeutically effective amount of each compound. The ability of a therapeutic agent or a combination to therapeutic agents to promote disease regression can be evaluated using a variety of methods known to the skilled practitioner, such as in human subjects during clinical trials, in animal model systems predictive of efficacy in humans, or by assaying the activity of the agent in in vitro assays.

The phrase “pharmaceutically acceptable” indicates that the substance or composition must be compatible chemically and/or toxicologically with the other ingredients comprising a formulation, and/or the subject (e.g., mammal) being treated therewith.

As used herein, “polymorphs” refer to distinct solids sharing the same molecular formula, yet each polymorph may have distinct solid state physical properties. A single compound may give rise to a variety of polymorphic forms where each form has different and distinct solid state physical properties, such as different solubility profiles, melting point temperatures, flowability, dissolution rates and/or different X-ray diffraction peaks. These practical physical characteristics are influenced by the conformation and orientation of molecules in the unit cell, which defines a particular polymorphic form of a substance. Polymorphic forms of a compound can be distinguished in a laboratory by X-ray diffraction spectroscopy, such as X-ray powder diffraction (“XRPD”), and by other methods, such as infrared spectrometry. Additionally, polymorphic forms of the same drug substance or active pharmaceutical ingredient can be administered by itself or formulated as a drug product (pharmaceutical composition) and are well known in the pharmaceutical art to affect, for example, the solubility, stability, flowability, tractability and compressibility of drug substances and the safety and efficacy of drug products. For more, see Hilfiker, Rolf (ed.), Polymorphism in the Pharmaceutical Industry. Weinheim, Germany: Wiley-VCH 2006.

As used herein, the term “amorphous” means a solid in a solid state that is a non-crystalline state. Amorphous solids generally possess crystal-like short range molecular arrangement, but no long range order of molecular packing as found in crystalline solids. The solid state form of a solid may be determined by polarized light microscopy, X-ray powder diffraction (“XRPD”), differential scanning calorimetry (“DSC”), or other standard techniques known to those of skill in the art.

As used herein, the term “crystalline” means a solid in a solid state having a regularly repeating arrangement of molecules or external face planes. The solid state form of a solid may be determined by polarized light microscopy, X-ray powder diffraction (“XRPD”), differential scanning calorimetry (“DSC”), or other standard techniques known to those of skill in the art. Accordingly, the term “crystalline purity,” as used herein, means the percentage of a certain crystalline polymorph of atrasentan or pharmaceutically acceptable salt thereof in a sample that may contain amorphous atrasentan, or a pharmaceutically acceptable salt thereof, one or more additional crystalline polymorphs of atrasentan, or a pharmaceutically acceptable salt thereof, or mixtures thereof. When a crystalline polymorph of atrasentan, or a pharmaceutically acceptable salt thereof is described as having “substantial crystalline purity”, it means the polymorph is substantially free (e.g., contains <10%, <5%, <2%, <1%, <0.5%, <0.1%, or <0.05%) of other polymorphs (amorphous and/or crystalline).

The term “chemical purity,” as used herein, means percentage of a particular compound (e.g., atrasentan, or a pharmaceutically acceptable salt thereof) in a sample. Accordingly, atrasentan, or a pharmaceutically acceptable salt thereof, and compositions comprising or made therefrom may contain one or more impurity, including but not limited to: water, ethyl acetate, ethanol, (2R,3R,4S)-2-(4-methoxyphenyl)-4-(1,3-benzodioxol-5-yl)-1-(N-(n-butyl)aminocarbonylmethyl)pyrrolidine-3-carboxylic acid, (2R,3R,4S)-2-(4-methoxyphenyl)-4-(1,3-benzodioxol-5-yl)-1-((N-(n-butyl)-N-ethyl)aminocarbonylmethyl)pyrrolidine-3-carboxylic acid, (2R,4S)-2-(4-methoxyphenyl)-4-(1,3-benzodioxol-5-yl)-1-(N,N-di(n-butyl)aminocarbonylmethyl)pyrrolidine, or ethyl (2R,3R,4S)-2-(4-methoxyphenyl)-4-(1,3-benzodioxol-5-yl)-1-(N,N-di(n-butyl)aminocarbonylmethyl)pyrrolidine-3-carboxylate. When a sample of atrasentan, or a pharmaceutically acceptable salt thereof is described as having “substantial purity”, the sample is substantially free of impurities (e.g., contains <10%, <5%, <2%, <1%, <0.5%, <0.1%, or <0.05%).

The term “diastereomeric excess,” as used herein, means the amount of one diastereomer of a compound (e.g., atrasentan, or a pharmaceutically acceptable salt thereof) in a mixture which may have other diastereomers of the same compound in the mixture. The term “substantial diastereomeric purity,” as used herein, means diastereomeric excess greater than about 90%, 95%, 99%, 99.5%, 99.9%, or 100%.

As used herein, the term “pharmaceutically acceptable carrier” refers to a substance that aids the administration of an active agent to a cell, an organism, or a subject. “Pharmaceutically acceptable carrier” refers to a carrier or excipient that can be included in the compositions of the disclosure and that causes no significant adverse toxicological effect on the subject. Non-limiting examples of pharmaceutically acceptable carriers include water, NaCl, normal saline solutions, lactated Ringer's, normal sucrose, normal glucose, binders, fillers, disintegrants, lubricants, coatings, sweeteners, flavors and colors, liposomes, dispersion media, microcapsules, cationic lipid carriers, isotonic and absorption delaying agents, and the like. The carrier may also be substances for providing the formulation with stability, sterility and isotonicity (e.g., antimicrobial preservatives, antioxidants, chelating agents and buffers), for preventing the action of microorganisms (e.g. antimicrobial and antifungal agents, such as parabens, chlorobutanol, phenol, sorbic acid and the like) or for providing the formulation with an edible flavor etc. In some instances, the carrier is an agent that facilitates the delivery of a small molecule drug or antibody to a target cell or tissue. One of skill in the art will recognize that other pharmaceutical carriers are useful in the present disclosure.

The term “SGLT-2 inhibitor” as used herein refers to a compound that inhibits the Sodium Glucose Co-Transporter-2 (SGLT-2). SGLT-2 inhibitors disrupt reabsorption of glucose by the kidneys and thus exert a glucose-lowering effect. By enhancing glucosuria, independently of insulin, SLGT-2 inhibitors have been shown to treat Type 2 diabetes and to improve cardiovascular outcomes. See Wright, 2001, Am. J. Physiol. Renal Physiol. 280:F10; and Scheen, 2018, Circ. Res. 122:1439. In some embodiments, the term “SGLT-2 inhibitor” refers to compounds whose primary effect is inhibition of SGLT-2, but is not limited to compounds that only inhibit SGLT-2, thus including compounds that have other activities in addition to SGLT-2 inhibition (e.g., SGLT-1 inhibition). In some embodiments, SGLT-2 inhibitors include compounds that are approved as SGLT-2 inhibitors by a regulatory agency such as the FDA or EMA. Non-limiting examples of SGLT-2 inhibitors include bexagliflozin, canagliflozin (INVOKANA®), dapagliflozin (FARXIGA®), empagliflozin (JARDIANCE®), ertugliflozin (STEGLATRO™), ipragliflozin (SUGLAT®), luseogliflozin (LUSEFI®), remogliflozin, serfliflozin, licofliglozin, sotagliflozin (ZYNQUISTA™), and tofogliflozin.

In some embodiments, the SGLT-2 inhibitors include, but are not limited to dapagliflozin, canagliflozin, ipragliflozin, empaglifozin, bexagliflozin, licogliflozin, janagliflozin (XZP-5695), tofogliflozin, ertugliflozin, henagliflozin (SHR-3824), enavogliflozin (DWP-16001), TA-1887 (3-(4-cyclopropylbenzyl)-4-fluoro-1-(β-D-glucopyranosyl)-1H-indole), indole-N-glycoside 18 (3-(4-ethylbenzyl)-1-(β-D-glucopyranosyl)-1H-indole), sotagliflozin, luseogliflozin, sergliflozin etabonate (ethyl carbonate), remogliflozin, remogliflozin etabonate, and T-1095 (((2R,3S,4S,5R,6S)-6-(2-(3-(benzofuran-5-yl)propanoyl)-3-hydroxy-5-methylphenoxy)-3,4,5-trihydroxytetrahydro-2H-pyran-2-yl) etabonate).

In some embodiments, the SGLT-2 inhibitors include C-glycosides such as dapagliflozin, canagliflozin, ipragliflozin, empaglifozin, bexagliflozin, licogliflozin, janagliflozin (XZP-5695), tofogliflozin, ertugliflozin, henagliflozin (SHR-3824), enavogliflozin (DWP-16001). In some embodiments, the SGLT-2 inhibitors include C-glycosides with a bicyclic or spiro pyran group, such as tofogliflozin, ertugliflozin, and henagliflozin (SHR-3824). In some embodiments, the SGLT-2 inhibitors include C-glycosides that do not have a bicyclic or spiro pyran group, such as dapagliflozin, canagliflozin, ipragliflozin, empaglifozin, bexagliflozin, licogliflozin, janagliflozin (XZP-5695), and enavogliflozin (DWP-16001).

In some embodiments, the SGLT-2 inhibitors include N-glycosides such as TA-1887 (3-(4-cyclopropylbenzyl)-4-fluoro-1-(β-D-glucopyranosyl)-1H-indole) and indole-N-glycoside 18 (3-(4-ethylbenzyl)-1-(β-D-glucopyranosyl)-1H-indole).

In some embodiments, the SGLT-2 inhibitors include 2-methylthio-C-glycosides, such as sotagliflozin.

In some embodiments, the SGLT-2 inhibitors include thiopyran-C-glycosides, such as luseogliflozin.

In some embodiments, the SGLT-2 inhibitors include O-glycosides and O-glycoside prodrugs, such as sergliflozin etabonate (ethyl carbonate), remogliflozin, remogliflozin etabonate, and T-1095 (((2R,3S,4S,5R,6S)-6-(2-(3-(benzofuran-5-yl)propanoyl)-3-hydroxy-5-methylphenoxy)-3,4,5-trihydroxytetrahydro-2H-pyran-2-yl) etabonate).

In some embodiments, an SGLT-2 inhibitor, as defined herein, includes any compound exhibiting SGLT-2 inhibition activity. In some embodiments, an SGLT-2 inhibitor is selective for SGLT-2 over SGLT-1, for example, by having about 2-fold, about 5-fold, about 10-fold, about 20-fold, about 50-fold, about 100-fold, about 200-fold, about 300-fold, about 400-fold, about 500-fold, about 750-fold, about 1,000-fold, about 1,250-fold, about 1,500-fold, about 1,750-fold, about 2,000-fold, about 2,500-fold, or any value in between, greater activity against SGLT-2 than against SGLT-1. Exemplary SGLT-2 inhibitors can exhibit inhibition activity (IC₅₀) against SGLT-2 of less than about 1000 nM, less than about 500 nM, less than about 200 nM, less than about 100 nM, less than about 50 nM, less than about 25 nM, less than about 10 nM, or less than about 1 nM as measured in an assay as described herein. In some embodiments, SGLT-2 inhibitors can exhibit inhibition activity (IC₅₀) against SGLT-2 of less than about 25 nM, less than about 10 nM, less than about 5 nM, or less than about 1 nM as measured in an assay as provided herein. An exemplary assay for determining SGLT-2 inhibitory activity is described in Ryan, et al., Kidney International, Vol. 45, pp. 48-57 (1994). Briefly, CHO cells are stably transfected with cDNA encoding human SGLT-2 (GenBank #M95549). Cells are washed and then incubated with 10 μM [¹⁴C]alpha-methyl glucopyranoside (AMG), and 10 μM inhibitor. The uptake of [¹⁴C]AMG is quenched with cold buffer containing phlorizin, and cells are lysed. Suitable reagents are then used to quantify the uptake of [¹⁴C]AMG.

SGLT-2 inhibitors include pharmaceutically acceptable salts, solvates, complexes, and salts of solvates thereof, for example, “dapagliflozin” includes salts of dapagliflozin (such as the hydrochloride salt) as well as solvates (such as the propylene glycol hydrate); likewise, “canagliflozin” includes solvates (such as canagliflozin hemihydrate) and salts of solvates (such as the hydrochloride salt of the hydrate). Similarly, henagliflozin (SHR-3824) and dapagliflozin include complexes (such as the complexes henagliflozin proline and dapagliflozin proline, respectively).

As used herein, when a subject is described as having “controlled serum glucose levels”, it means the subject has a serum glucose level within the normal or healthy ranges. In some embodiments, the subject has a fasting serum glucose level of about 70 mg/dL to about 130 mg/dL. For example, the subject can be determined to have a fasting serum glucose level of below about 130 mg/dL, below about 125 mg/dL, below about 120 mg/dL, below about 115 mg/dL, below about 110 mg/dL, below about 105 mg/dL, below about 100 mg/dL, below about 95 mg/dL, below about 90 mg/dL, below about 85 mg/dL, below about 80 mg/dL, or below about 75 mg/dL.

As used in the methods described herein, the term “reducing” refers to a reduction in the indicated parameter relative to the baseline measurement (or measurements) of the same parameter in the subject taken prior to the initiation of administration (the first administration) of a therapeutic agent or combination of therapeutic agents (e.g., atrasentan, or a pharmaceutically acceptable salt thereof, and a SGLT-2 inhibitor), or a reduction in the indicated parameter relative to the baseline measurement (or measurements) of the same parameter in a healthy subject (for example, a subject that does not have DKD). Similarly, the term “increasing,” as used herein, refers to an increase in the indicated parameter relative to the baseline measurement (or measurements) of the same parameter in the subject taken prior to the initiation of administration of a therapeutic agent or combination of therapeutic agents (e.g., atrasentan, or a pharmaceutically acceptable salt thereof, and a SGLT-2 inhibitor), or an increase in the indicated parameter relative to the baseline measurement (or measurements) of the same parameter in a healthy subject (e.g., a subject that does not have DKD).

The term “glomerular filtration rate (GFR)” is defined as the volume of fluid filtered from the renal (kidney) glomerular capillaries into the Bowman's capsule per unit time. It is indicative of overall kidney function. The glomerular filtration rate (GFR) can be calculated by measuring any chemical that has a steady level in the blood, and is freely filtered but neither reabsorbed nor secreted by the kidneys. The rate therefore measured is the quantity of the substance in the urine that originated from a calculable volume of blood. The GFR is typically recorded in units of volume per time, e.g., milliliters per minute and the formula below can be used: GFR=(Urine ConcentrationxUrine Volume)/Plasma Concentration. The GFR can be determined by injecting inulin into the plasma. Since inulin is neither reabsorbed nor secreted by the kidney after glomerular filtration, its rate of excretion is directly proportional to the rate of filtration of water and solutes across the glomerular filter. A normal value for a healthy human subject is: GFR=90-125 mL/min/1.73 m², in particular GFR=100-125 mL/min/1.73 m². Other principles to determine GFR involve measuring 51Cr-EDTA, [¹²⁵I]iothalamate or iohexol. The term “estimated glomerular filtration rate (eGFR)” is defined as derived at screening from serum creatinine values based on e.g., the Chronic Kidney Disease Epidemiology Collaboration (CKD-EPI) equation, the Cockcroft-Gault formula or the Modification of Diet in Renal Disease (MDRD) formula, which are all known in the art. “Stabilizing eGFR” as used herein means reducing the rate of decrease of eGFR and/or attenuating the rate of decline of eGFR. For example, the rate of decline of eGFR can be attenuated by at least about 20%; by at least about 30%; by at least about 40%; by at least about 50%; by at least about 60%; by at least about 70%; by at least about 80%; by at least about 90%; or by at least about 95%; or any value in between, after treatment with atrasentan, or a pharmaceutically acceptable salt thereof, and a SGLT-2 inhibitor, according to context. This attenuation can be after treatment, for example, for about 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between. In some embodiments, the subject has been treated with atrasentan, or a pharmaceutically acceptable salt thereof, and a SGLT-2 inhibitor for about 15 days to about 30 days. In some embodiments, the subject has been treated with atrasentan, or a pharmaceutically acceptable salt thereof, and a SGLT-2 inhibitor for about 6 months to about 1 year.

“ESRD” is the abbreviation for end-stage renal disease. As used herein, the onset of ESRD is defined as the time point when the subject has an eGFR of below about 15 mL/min/1.73 m² and/or when the subject has initiated chronic dialysis.

As used herein, when a subject is described to “maintain a potassium level within the normal physiologic range”, the subject has a blood potassium level of from about 3.5 mEq/L to about 5.2 mEq/L.

As used herein, when a subject is described to “maintain a sodium level within the normal physiologic range”, the subject has a blood sodium level of from about 135 to about 145 mEq/L.

As used herein, an “improved treatment outcome” refers to any beneficial or desired result of treatment, including, but not limited to reducing the risk of developing ESRD, reducing the risk of kidney transplantation, reducing the risk of dialysis, reducing the frequency of dialysis, reducing the risk of kidney transplantation, increasing renal function, and reducing the risk of death due to renal failure.

As used herein, the term “proteinuria” refers to the presence of protein in the urine in excess of normal levels. “Proteinuria” includes “albuminuria” and “microalbuminuria” and. Normal human levels of protein appear in the urine in the range of about 0 mg/L to about 30 mg/L, although for any given urine sample, the level may reach about 80 mg/L. For a 24 hour urine collection, normal human levels of urinary protein are in the range of about 0 mg to about 150 mg. Proteinuria is indicated by a urinary albumin/creatinine ratio (UACR) of greater than about 30 mg/g. Typically, the UACR value in mg/g approximates the albumin excretion by the subject in mg/day. Proteinuria, including albuminuria and microalbuminuria, often leads to or is indicative of a disease, but is not limited to production of a disease. Proteinuria is intended to encompass all forms of proteinuria, including but not limited to physiological proteinuria; functional proteinuria; and athletic proteinuria, which relates to a form of functional proteinuria following excessive muscular exertion. Further, proteinuria covers benign proteinuria (also known as “essential” proteinuria), which refers to types or proteinuria that are not the result of pathologic changes in the kidneys. Proteinuria also covers pathologic proteinuria, for example levels of protein in the urine greater than normal physiological levels.

As used herein, the term “albuminuria” (also known as “macroalbuminuria”) refers to the presence of albumin in the urine in excess of normal levels. Since urinary protein is predominantly albumin, normal human levels of UACR are in the range of about 0 mg/g to about 30 mg/g. As used herein, the term “microalbuminuria” refers to the presence of albumin in the urine, excreted at a rate of about 20 μg/min to about 200 μg/min or at a level of about 30 mg/L to about 300 mg/L in humans. When defined by the UACR, “microalbuminuria” refers to a UACR of greater than about 30 mg/g, or a UACR of about 3.5 mg/g or greater for women and about 2.5 mg/g or greater for men. Microalbuminuria is often an early warning of kidney disease, but can also be present for other reasons.

The term “synergy” or “synergistic” is used herein to mean that the effect of a combination of two or more therapeutic agents is greater than the sum of the effects of each agent when administered alone. See, e.g., Chou and Talalay, Advances in Enzyme Regulation (1984), 22, 27-55. A “synergistically effective amount” is an amount of the combination of the two or more therapeutic agents that results in a synergistic effect (as “synergistic” is defined herein). In some embodiments, a synergistically effective amount of a combination may be therapeutically effective even when one or more of the compounds in the combination is administered at a dose that would be sub-therapeutic when the compound is administered alone.

It will be appreciated that different concentrations of each agent may be employed for various art-recognized factors, for example, a subject's height, weight, sex, age and medical history. Exemplary synergistic effects includes, but are not limited to, enhanced therapeutic efficacy, decreased dosage at equal or increased level of efficacy, reduced or delayed development of drug resistance, and simultaneous enhancement or equal therapeutic actions (e.g., the same therapeutic effect as at least one of the therapeutic agents) and a reduction of unwanted drug effects (e.g. side effects and adverse events) of at least one of the therapeutic agents.

In some embodiments, “synergistic effect” as used herein refers to a combination of atrasentan, or a pharmaceutically acceptable salt thereof, and a SGLT-2 inhibitor, producing an effect, for example, any of the beneficial or desired results including clinical results as described herein, which is greater than the sum of the effects observed when atrasentan, or a pharmaceutically acceptable salt thereof, and a SGLT-2 inhibitor are administered alone, and/or a reduction in the occurrence and/or severity of an unwanted drug effect, side effect, or adverse event. Such clinical results include, but are not limited to, treating DKD, reducing UACR, reducing weight gain or fluid retention, decreasing proteinuria (e.g., albuminuria), reducing the rate of decrease of eGFR, stabilizing eGFR, and/or delaying the onset of ESRD. Such unwanted drug effects, side effects, or adverse events include, but are not limited to, congestive heart failure, fluid retention, anemia, increased brain natriuretic peptide (BNP) levels, acute sodium retention, and acute increases in creatinine levels. In some embodiments, the fluid retention is associated with a weight gain of greater than about 3 kg. In some embodiments, the increased BNP levels are greater than about 300 pg/mL. In some embodiments, beneficial or desired result and/or the unwanted drug effect, side effect, or adverse event is associated with or observed in monotherapy of atrasentan, or a pharmaceutically acceptable salt thereof, or monotherapy of a SGLT-2 inhibitor.

As described herein, any concentration range, percentage range, ratio range, or integer range is to be understood to include the value of any integer within the recited range and, when appropriate, fractions thereof (such as one tenth and one hundredth of an integer), unless otherwise indicated.

Unless otherwise stated, any reference to an amount of atrasentan in this disclosure is based on the free equivalent weight of atrasentan. For example, 0.75 mg of atrasentan refers to 0.75 mg of atrasentan in the free form or an equivalent amount of a salt form of atrasentan.

Various aspects of the disclosure are described in further detail in the following subsections.

### B. Introduction

Most patients with diabetes have some form of kidney disease. In the early stages of diabetic kidney disease (DKD), small increases in urinary albumin (microalbuminuria) are detectable. As the disease progresses, urinary albumin increases and other measures of impaired kidney function, such as decreases in glomerular filtration rate (GFR), as appear. Increases in the level of brain (B-type) natriuretic protein are also detected in early stage DKD. Without treatment, patients with DKD will ultimately progress to ESRD. Current treatments for DKD include administration of angiotensin-converting enzyme (ACE) inhibitors, angiotensin II receptor blockers (ARBs) and diuretics. However, current therapies may not be sufficient for disease management and can cause adverse side effects of their own. Thus, there remains a need for additional treatment options for DKD patients.

### C. Methods of Treatment

In one aspect, provided herein is a method of treating diabetic kidney disease (DKD), comprising administering a therapeutically effective amount of atrasentan, or a pharmaceutically acceptable salt thereof, and a therapeutically effective amount of a SGLT-2 inhibitor to a subject in need thereof.

In another aspect, provided is a method of delaying progressive renal function decline in a subject having DKD, comprising administering a therapeutically effective amount of atrasentan, or a pharmaceutically acceptable salt thereof, and a therapeutically effective amount of a SGLT-2 inhibitor to the subject.

In another aspect, provided is a method of treating chronic kidney disease associated with diabetes, comprising administering a therapeutically effective amount of atrasentan, or a pharmaceutically acceptable salt thereof, and a therapeutically effective amount of a SGLT-2 inhibitor to a subject in need thereof.

In another aspect, provided is a method of improving treatment outcome of a subject having diabetic kidney disease, comprising administering a therapeutically effective amount of atrasentan, or a pharmaceutically acceptable salt thereof, and a therapeutically effective amount of a SGLT-2 inhibitor to a subject in need thereof.

In another aspect, provided is a method of reducing the UACR of a subject having diabetic kidney disease in a subject, comprising administering a therapeutically effective amount of atrasentan, or a pharmaceutically acceptable salt thereof, and a therapeutically effective amount of a SGLT-2 inhibitor to the subject; wherein the UACR after administration of the atrasentan, or a pharmaceutically acceptable salt thereof, and the SGLT-2 inhibitor is less than the UACR prior to administration of the atrasentan, or a pharmaceutically acceptable salt thereof, and the SGLT-2 inhibitor.

In another aspect, provided is a method of reducing fluid retention in a subject having diabetic kidney disease, comprising administering a therapeutically effective amount of atrasentan, or a pharmaceutically acceptable salt thereof, and a therapeutically effective amount of a SGLT-2 inhibitor to the subject; wherein fluid retention or weigh gain after administration of the atrasentan, or a pharmaceutically acceptable salt thereof, and the SGLT-2 inhibitor is less than fluid retention or body weight prior to administration of the atrasentan, or a pharmaceutically acceptable salt thereof, and the SGLT-2 inhibitor.

In another aspect, provided is a method of reducing fluid retention in a subject having diabetic kidney disease, comprising administering a therapeutically effective amount of atrasentan, or a pharmaceutically acceptable salt thereof, and a therapeutically effective amount of a SGLT-2 inhibitor, to the subject in need thereof; wherein fluid retention after administration of the SGLT-2 inhibitor is less than fluid retention prior to the administration of the SGLT-2 inhibitor.

In another aspect, provided is a method of reducing fluid retention in a subject having diabetic kidney disease during treatment with atrasentan, or a pharmaceutically acceptable salt thereof, comprising administering a therapeutically effective amount of a SGLT-2 inhibitor, to a subject in need thereof; wherein fluid retention after administration of the SGLT-2 inhibitor is less than fluid retention prior to administration of the SGLT-2 inhibitor.

In another aspect, provided is a method of reducing fluid retention in a subject having diabetic kidney disease, comprising administering a therapeutically effective amount of atrasentan, or a pharmaceutically acceptable salt thereof, and a therapeutically effective amount of a SGLT-2 inhibitor, to the subject in need thereof; wherein fluid retention after administration of the atrasentan, or a pharmaceutically acceptable salt thereof, is less than fluid retention prior to the administration of atrasentan, or a pharmaceutically acceptable salt thereof.

In another aspect, provided is a method of reducing fluid retention in a subject having diabetic kidney disease during treatment with a SGLT-2 inhibitor, comprising administering a therapeutically effective atrasentan, or a pharmaceutically acceptable salt thereof, to the subject in need thereof; wherein fluid retention after administration of the atrasentan, or a pharmaceutically acceptable salt thereof, is less than fluid retention prior to administration of the atrasentan, or a pharmaceutically acceptable salt thereof.

In another aspect, provided is a method of reducing a BNP level in a subject having diabetic kidney disease in a subject, comprising administering a therapeutically effective amount of atrasentan, or a pharmaceutically acceptable salt thereof, and a therapeutically effective amount of a SGLT-2 inhibitor to the subject; wherein the BNP level after administration of the atrasentan, or a pharmaceutically acceptable salt thereof, and the SGLT-2 inhibitor is less than the BNP level prior to administration of the atrasentan, or a pharmaceutically acceptable salt thereof, and the SGLT-2 inhibitor.

In another aspect, provided is a method of reducing the rate of decrease of eGFR of a subject having diabetic kidney disease in a subject, comprising administering a therapeutically effective amount of atrasentan, or a pharmaceutically acceptable salt thereof, and a therapeutically effective amount of a SGLT-2 inhibitor to the subject; wherein the rate of decrease of eGFR after administration of the atrasentan, or a pharmaceutically acceptable salt thereof, and the SGLT-2 inhibitor is less than the rate of decrease of eGFR prior to administration of the atrasentan, or a pharmaceutically acceptable salt thereof, and the SGLT-2 inhibitor.

In another aspect, provided is a method of stabilizing estimated glomerular filtration rate (eGFR) of a subject having diabetic kidney disease, comprising administering a therapeutically effective amount of atrasentan, or a pharmaceutically acceptable salt thereof, and a therapeutically effective amount of a SGLT-2 inhibitor to subject in need thereof; wherein the eGFR of the subject following administration of the atrasentan, or a pharmaceutically acceptable salt thereof, and the SGLT-2 inhibitor is stabilized eGFR relative to a subject not administered the atrasentan, or a pharmaceutically acceptable salt thereof, and the SGLT-2 inhibitor.

In another aspect, provided is a method of improving treatment outcome in a subject having diabetic kidney disease, comprising administering a synergistically effective amount of atrasentan, or a pharmaceutically acceptable salt thereof, and a synergistically effective amount of a SGLT-2 inhibitor to a subject in need thereof.

In some embodiments, the urine albumin to creatinine ratio (UACR) of the subject following administration of atrasentan, or a pharmaceutically acceptable salt thereof, and the SGLT-2 inhibitor decreases relative to the UACR of a subject not administered the atrasentan, or a pharmaceutically acceptable salt thereof, and the SGLT-2 inhibitor. In some embodiments, body weight or fluid retention of the subject following administration of the atrasentan, or a pharmaceutically acceptable salt thereof, and the SGLT-2 inhibitor decreases relative to body weight or fluid retention of a subject not administered the atrasentan, or a pharmaceutically acceptable salt thereof, and the SGLT-2 inhibitor. In some embodiments, the rate of decrease the estimated glomerular filtration rate (eGFR) of the subject following administration of the atrasentan, or a pharmaceutically acceptable salt thereof, and the SGLT-2 inhibitor decreases relative to the eGFR of a subject not administered the atrasentan, or a pharmaceutically acceptable salt thereof, and the SGLT-2 inhibitor. In some embodiments, the B-type (or brain) natriuretic peptide (BNP) level in the subject following administration of the atrasentan, or a pharmaceutically acceptable salt thereof, and the SGLT-2 inhibitor decreases relative to the BNP level in a subject not administered the atrasentan, or a pharmaceutically acceptable salt thereof, and the SGLT-2 inhibitor. In some embodiments, at least one, at least two, or at least three of the subject's UACR, body weight or fluid retention, BNP level and rate of decrease of eGFR are decreased following administration of the atrasentan, or a pharmaceutically acceptable salt thereof, and the SGLT-2 inhibitor, decreases relative to the subject's UACR, body weight or fluid retention, BNP level, and rate of decrease of eGFR in the subject relative to a subject not administered the atrasentan, or a pharmaceutically acceptable salt thereof, and the SGLT-2 inhibitor. In some embodiments, at least one of the subject's UACR, body weight or fluid retention, BNP level, and rate of decrease of eGFR are decreased following administration of the atrasentan, or a pharmaceutically acceptable salt thereof, and the SGLT-2 inhibitor, decreases relative to the subject's UACR, body weight or fluid retention, BNP level and rate of decrease of eGFR in the subject relative to a subject not administered the atrasentan, or a pharmaceutically acceptable salt thereof, and the SGLT-2 inhibitor. In some embodiments, at least two of the subject's UACR, body weight or fluid retention, BNP level, and rate of decrease of eGFR are decreased following administration of the atrasentan, or a pharmaceutically acceptable salt thereof, and the SGLT-2 inhibitor, decreases relative to the subject's UACR, body weight or fluid retention, BNP level and rate of decrease of eGFR in the subject relative to a subject not administered the atrasentan, or a pharmaceutically acceptable salt thereof, and the SGLT-2 inhibitor. In some embodiments, at least three of the subject's UACR, body weight or fluid retention, BNP level and rate of decrease of eGFR are decreased following administration of the atrasentan, or a pharmaceutically acceptable salt thereof, and the SGLT-2 inhibitor, decreases relative to the subject's UACR, body weight or fluid retention, BNP level, and rate of decrease of eGFR in the subject relative to a subject not administered the atrasentan, or a pharmaceutically acceptable salt thereof, and the SGLT-2 inhibitor. In some embodiments, the subject's UACR, body weight or fluid retention, BNP level, and rate of decrease of eGFR are decreased following administration of the atrasentan, or a pharmaceutically acceptable salt thereof, and the SGLT-2 inhibitor, decreases relative to the subject's UACR, body weight or fluid retention, BNP level and rate of decrease of eGFR in the subject relative to a subject not administered the atrasentan, or a pharmaceutically acceptable salt thereof, and the SGLT-2 inhibitor. In some embodiments, the subject's UACR, body weight or fluid retention, and BNP level, are decreased following administration of the atrasentan, or a pharmaceutically acceptable salt thereof, and the SGLT-2 inhibitor, wherein the decrease is relative to the subject's UACR, body weight or fluid retention, and BNP level in the subject relative to a subject not administered the atrasentan, or a pharmaceutically acceptable salt thereof, and the SGLT-2 inhibitor.

In some embodiments, the BNP level of a subject begin administered atrasentan, or a pharmaceutically acceptable salt thereof, following administration of the SGLT-2 inhibitor decreases relative to the BNP level of a subject not administered the SGLT-2 inhibitor.

In the embodiments described herein, when a subject is being administered a therapeutic agent (for example, atrasentan, or a pharmaceutically acceptable salt thereof) and is then administered another compound (for example, a SGLT-2 inhibitor) “during treatment” with the therapeutic agent, the subject will be administered both compounds (for example, atrasentan, or a pharmaceutically acceptable salt thereof, and a SGLT-2 inhibitor), whereas previously they were only administered one compound (for example, atrasentan, or a pharmaceutically acceptable salt thereof).

In some embodiments, the subject has Type 2 diabetes. In some embodiments, the subject has Type 1 diabetes. In some embodiments, the subject has been previously diagnosed with Type 2 diabetes. In some embodiments, the subject has been previously diagnosed with Type 1 diabetes. In some embodiments, the subject is currently suffering from Type 2 diabetes. In some embodiments, the subject is currently suffering from Type 1 diabetes.

In some embodiments, the subject had previously been administered atrasentan, or a pharmaceutically acceptable salt thereof. In some embodiments, the subject had previously been administered a SGLT-2 inhibitor.

In some further embodiments of the embodiments described above, the subject has not been previously diagnosed with one or more of IgA nephropathy, HIV/AIDS, HIV-related nephropathy, prostate cancer, or acute kidney failure. In some embodiments, the subject has not been previously diagnosed with any of IgA nephropathy, HIV/AIDS, HIV-related nephropathy, prostate cancer, or acute kidney failure. In some embodiments, the subject has not been previously diagnosed with IgA nephropathy. In some embodiments, the subject has not been previously diagnosed with HIV/AIDS. In some embodiments, the subject has not been previously diagnosed with HIV-related nephropathy. In some embodiments, the subject has not been previously diagnosed with prostate cancer. In some embodiments, the subject has not been previously diagnosed with acute kidney failure.

In some further embodiments of the embodiments described above, the subject has not been previously diagnosed with one or more of IgA nephropathy, HIV/AIDS, HIV-related nephropathy, cancer, or acute kidney failure. In some embodiments, the subject has not been previously diagnosed with cancer.

In some further embodiments of the embodiments described above, the subject is not currently diagnosed with cancer. In some embodiments, the subject is not currently being treated for cancer. In some embodiments, the cancer is lung cancer or prostate cancer.

In some further embodiments of the embodiments described above, the subject does not have one or more of IgA nephropathy, HIV/AIDS, HIV-related nephropathy, cancer, or acute kidney failure. In some embodiments, the subject does not have one or more of IgA nephropathy, HIV/AIDS, HIV-related nephropathy, prostate cancer, or acute kidney failure. In some embodiments, the subject does not have any of IgA nephropathy, HIV/AIDS, HIV-related nephropathy, cancer, or acute kidney failure. In some embodiments, the subject does not have any of IgA nephropathy, HIV/AIDS, HIV-related nephropathy, prostate cancer, or acute kidney failure. In some embodiments, the subject does not have IgA nephropathy. In some embodiments, the subject does not have HIV/AIDS. In some embodiments, the subject does not have HIV-related nephropathy. In some embodiments, the subject does not have cancer. In some embodiments, the cancer is prostate cancer. In some embodiments, the cancer is lung cancer. In some embodiments, the subject does not have acute kidney failure.

In some further embodiments of the embodiments described above, the subject is not being treated for one or more of IgA nephropathy, HIV/AIDS, HIV-related nephropathy, cancer, or acute kidney failure. In some embodiments, the subject is not being treated for one or more of IgA nephropathy, HIV/AIDS, HIV-related nephropathy, prostate cancer, or acute kidney failure. In some embodiments, the subject is not being treated for any of IgA nephropathy, HIV/AIDS, HIV-related nephropathy, cancer, or acute kidney failure. In some embodiments, the subject is not being treated for any of IgA nephropathy, HIV/AIDS, HIV-related nephropathy, prostate cancer, or acute kidney failure. In some embodiments, the subject is not being treated for IgA nephropathy. In some embodiments, the subject is not being treated for HIV/AIDS. In some embodiments, the subject is not being treated for HIV-related nephropathy. In some embodiments, the subject is not being treated for cancer. In some embodiments, the subject is not being treated for prostate cancer. In some embodiments, the subject is not being treated for lung cancer. In some embodiments, the subject is not being treated for acute kidney failure.

In some further embodiments of the embodiments described above, the subject has been determined to have controlled serum glucose levels prior to treatment with atrasentan, or a pharmaceutically acceptable salt thereof, and a SGLT-2 inhibitor. For example, the subject has been determined to have a fasting serum glucose level of below about 130 mg/dL, below about 125 mg/dL, below about 120 mg/dL, below about 115 mg/dL, below about 110 mg/dL, below about 105 mg/dL, below about 100 mg/dL, below about 95 mg/dL, below about 90 mg/dL, below about 85 mg/dL, below about 80 mg/dL, or below about 75 mg/dL, or any value in between. In some embodiments, the subject has been determined to have a fasting serum glucose level of below about 80 mg/dL to about 120 mg/dL, below about 80 mg/dL to about 100 mg/dL, below about 90 mg/dL to about 120 mg/dL, below about 100 mg/dL to about 120 mg/dL, or any value in between.

### Subject Selection

The subject having diabetic kidney disease (DKD) as described anywhere herein can be diagnosed using one or more methods known in the art. Non-limiting examples include: detection of microalbuminuria following a diagnosis of diabetes, increases in proteinuria following a diagnosis of diabetes, albuminuria following a diagnosis of diabetes, renal biopsy following a diagnosis of diabetes, and decrease in eGFR following a diagnosis of diabetes.

In some embodiments, the subject has a UACR of about 300 mg/g to about 5,000 mg/g prior to the first administration of atrasentan, or a pharmaceutically acceptable salt thereof. For example, about 300 mg/g to about 2,000 mg/g, about 300 mg/g to about 1,000 mg/g, about 300 mg/g to about 500 mg/g, about 300 mg/g/to about 800 mg/g, about 500 mg/g to about 1,000 mg/g, about 750 mg/g to about 1,250 mg/g, about 1,000 mg/g to about 1,500 mg/g, about 1,250 mg/g to about 1,750 mg/g, about 1,500 mg/g to about 2,000 mg/g, about 1,750 mg/g to about 2,250 mg/g, about 2,000 mg/g to about 2,500 mg/g, about 2,250 mg/g to about 2,750 mg/g, about 2,500 mg/g to about 3,000 mg/g, about 2,750 mg/g to about 3,250 mg/g, about 3,000 mg/g to about 3,500 mg/g, about 3,250 mg/g to about 3,750 mg/g, about 3,500 mg/g to about 4,000 mg/g, about 3,750 mg/g to about 4,250 mg/g, about 4,000 mg/g to about 4,500 mg/g, about 4,250 mg/g to about 4,750 mg/g, about 4,500 mg/g to about 5,000 mg/g, or any value in between. In some embodiments, the subject has a UACR of about 300 mg/g, about 400 mg/g, about 500 mg/g, about 600 mg/g, about 700 mg/g, about 800 mg/g, about 900 mg/g, about 1,000 mg/g, about 1,100 mg/g, about 1,200 mg/g, about 1,300 mg/g, about 1,400 mg/g, about 1,500 mg/g, about 1,600 mg/g, about 1,700 mg/g, about 1,800 mg/g, about 1,900 mg/g, about 2,000 mg/g, about 2,500 mg/g, about 3,000 mg/g, about 3,500 mg/g, about 4,500 mg/g, about 5000 mg/g, or any value in between.

When the phrase “prior to the first administration” of a compound or compound(s) is used herein, this refers to the last measurement, or the average of last measurements, prior to the first administration of the compound or compound(s), unless a specific time period is specified (for example, within three months prior to the first administration).

In some embodiments, the subject has a brain natriuretic peptide (BNP) concentration of about 200 pg/mL or less, prior to the first administration of atrasentan, or a pharmaceutically acceptable salt thereof. In some embodiments, the subject has a BNP concentration of about 200 pg/mL or less, after the first administration of a SGLT-2 inhibitor. In some embodiments, the subject has a brain natriuretic peptide (BNP) concentration of about 200 pg/mL or less, prior to the first administration of atrasentan, or a pharmaceutically acceptable salt thereof, and the SGLT-2 inhibitor. For example, the subject has a brain natriuretic peptide (BNP) concentration of about 20 pg/mL to about 200 pg/mL, about 20 pg/mL to about 180 pg/mL, about 20 pg/mL to about 160 pg/mL, about 20 pg/mL to about 140 pg/mL, about 20 pg/mL to about 120 pg/mL, about 20 pg/mL to about 100 pg/mL, about 20 pg/mL to about 80 pg/mL, about 20 pg/mL to about 60 pg/mL, about 20 pg/mL to about 40 pg/mL, about 40 pg/mL to about 60 pg/mL, about 40 pg/mL to about 120 pg/mL, about 60 pg/mL to about 140 pg/mL, about 80 pg/mL to about 160 pg/mL, about 100 pg/mL to about 180 pg/mL, about 120 pg/mL to about 200 pg/mL, or any value in between.

In some embodiments, the subject has systolic blood pressure of about 110 mm Hg to about 180 mm Hg prior to the first administration of atrasentan, or a pharmaceutically acceptable salt thereof. In some embodiments, the subject has systolic blood pressure of about 110 mm Hg to about 180 mm Hg prior to the first administration of atrasentan, or a pharmaceutically acceptable salt thereof, and the SGLT-2 inhibitor. For example, the subject has systolic blood pressure of about 110 mm Hg to about 130 mm Hg, about 120 mm Hg to about 140 mm Hg, about 130 mm Hg to about 150 mm Hg, about 140 mm Hg to about 160 mm Hg, about 150 mm Hg to about 170 mm Hg, about 160 mm Hg to about 180 mm Hg, or any value in between.

In some embodiments, the subject has an eGFR of about 75 mL/min/1.73 m² to about 25 mL/min/1.73 m², prior to the first administration of atrasentan, or a pharmaceutically acceptable salt thereof. In some embodiments, the subject has an eGFR of about 75 mL/min/1.73 m² to about 25 mL/min/1.73 m², prior to the first administration of atrasentan, or a pharmaceutically acceptable salt thereof, and the SGLT-2 inhibitor. For example, the subject has an eGFR of about 75 mL/min/1.73 m² to about 55 mL/min/1.73 m², about 65 mL/min/1.73 m² to about 45 mL/min/1.73 m², about 55 mL/min/1.73 m² to about 35 mL/min/1.73 m², about 45 mL/min/1.73 m² to about 25 mL/min/1.73 m², about 60 mL/min/1.73 m² to about 25 mL/min/1.73 m², about 45 mL/min/1.73 m² to about 25 mL/min/1.73 m², or any value in between.

In some embodiments, the subject has a serum albumin level of at least about 25 g/L prior to the first administration of atrasentan, or a pharmaceutically acceptable salt thereof. In some embodiments, the subject has a serum albumin level of at least about 25 g/L prior to the first administration of atrasentan, or a pharmaceutically acceptable salt thereof, and the SGLT-2 inhibitor. For example, the subject has a serum albumin level of about 25 g/L to about 55 g/L, about 25 g/L to about 40 g/L, about 30 g/L to about 45 g/L, about 35 g/L to about 50 g/L, about 40 g/L to about 55 g/L, or any value in between, prior to the first administration of atrasentan, or a pharmaceutically acceptable salt thereof.

In some embodiments, the subject has been diagnosed with diabetes, wherein prior to the first administration of atrasentan, or a pharmaceutically acceptable salt thereof, the subject has (i) a UACR of about 300 mg/g to about 5,000 mg/g; (ii) a brain natriuretic peptide (BNP) concentration of about 200 pg/mL or less; and (iii) an eGFR of about 75 mL/min/1.73 m² to about 25 mL/min/1.73 m². In some embodiments, the subject has been diagnosed with diabetes, wherein prior to the first administration of atrasentan, or a pharmaceutically acceptable salt thereof, the subject has (i) a UACR of about 300 mg/g to about 5,000 mg/g; and (ii) a brain natriuretic peptide (BNP) concentration of about 200 pg/mL or less. In some embodiments, the subject has been diagnosed with diabetes, wherein prior to the first administration of atrasentan, or a pharmaceutically acceptable salt thereof, the subject has (i) a UACR of about 300 mg/g to about 5,000 mg/g; and (ii) an eGFR of about 75 mL/min/1.73 m² to about 25 mL/min/1.73 m². In some embodiments, the subject has been diagnosed with diabetes, wherein prior to the first administration of atrasentan, or a pharmaceutically acceptable salt thereof, the subject has (i) a brain natriuretic peptide (BNP) concentration of about 200 pg/mL or less; and (ii) an eGFR of about 75 mL/min/1.73 m² to about 25 mL/min/1.73 m². In some embodiments, the subject has been diagnosed with Type 1 diabetes. In some embodiments, the subject has been diagnosed with Type 2 diabetes.

In some embodiments, the subject has been diagnosed with diabetes, wherein prior to the first administration of atrasentan, or a pharmaceutically acceptable salt thereof, and the SGLT-2 inhibitor, and the subject has (i) a UACR of about 300 mg/g to about 5,000 mg/g; (ii) a brain natriuretic peptide (BNP) concentration of about 200 pg/mL or less; and (iii) an eGFR of about 75 mL/min/1.73 m² to about 25 mL/min/1.73 m². In some embodiments, the subject has been diagnosed with diabetes, wherein prior to the first administration of atrasentan, or a pharmaceutically acceptable salt thereof, and the SGLT-2 inhibitor, and the subject has (i) a UACR of about 300 mg/g to about 5,000 mg/g; and (ii) a brain natriuretic peptide (BNP) concentration of about 200 pg/mL or less. In some embodiments, the subject has been diagnosed with diabetes, wherein prior to the first administration of atrasentan, or a pharmaceutically acceptable salt thereof, and the SGLT-2 inhibitor, and the subject has (i) a UACR of about 300 mg/g to about 5,000 mg/g; and (ii) an eGFR of about 75 mL/min/1.73 m² to about 25 mL/min/1.73 m². In some embodiments, the subject has been diagnosed with diabetes, wherein prior to the first administration of atrasentan, or a pharmaceutically acceptable salt thereof, and the SGLT-2 inhibitor, and the subject has (i) a brain natriuretic peptide (BNP) concentration of about 200 pg/mL or less; and (ii) an eGFR of about 75 mL/min/1.73 m² to about 25 mL/min/1.73 m². In some embodiments, the subject has been diagnosed with diabetes, wherein prior to the first administration of atrasentan, or a pharmaceutically acceptable salt thereof, and the SGLT-2 inhibitor, the subject has (i) a UACR of about 300 mg/g to about 1,000 mg/g; and (ii) a brain natriuretic peptide (BNP) concentration of about 100 pg/mL or less. In some embodiments, the subject has been diagnosed with diabetes, wherein prior to the first administration of atrasentan, or a pharmaceutically acceptable salt thereof, and the SGLT-2 inhibitor, the subject has (i) a UACR of about 300 mg/g to about 1,000 mg/g; and (ii) an eGFR of about 60 mL/min/1.73 m² to about 25 mL/min/1.73 m². In some embodiments, the subject has been diagnosed with diabetes, wherein prior to the first administration of atrasentan, or a pharmaceutically acceptable salt thereof, and the SGLT-2 inhibitor, the subject has (i) a brain natriuretic peptide (BNP) concentration of about 100 pg/mL or less; and (ii) an eGFR of about 50 mL/min/1.73 m² to about 25 mL/min/1.73 m². In some embodiments, the subject has been diagnosed with Type 1 diabetes. In some embodiments, the subject has been diagnosed with Type 2 diabetes.

In some embodiments, the subject has an average fasting blood glucose level of about 80 mg/dL to about 200 mg/dL or more, (such as about 80 mg/dL to about 120 mg/dL, about 100 mg/dL to about 140 mg/dL, about 120 mg/dL to about 160 mg/dL, about 140 mg/dL to about 180 mg/dL, about 160 mg/dL to about 200 mg/dL, or any value in between) for at least about 3 months (e.g., at least about 4 months, at least about 5 months, at least about 6 months, at least about 7 months, at least about 8 months, at least about 9 months, at least about 10 months, at least about 11 months, at least about a year, at least about 1.5 years, or at least about 2 years) prior to the first administration of atrasentan, or a pharmaceutically acceptable salt thereof, or prior to the first administration of atrasentan, or a pharmaceutically acceptable salt thereof, and a SGLT-2 inhibitor. For example, the subject has an average fasting blood glucose level of about 80 mg/dL, about 90 mg/dL, about 100 mg/dL, about 110 mg/dL, about 120 mg/dL, about 130 mg/dL, about 140 mg/dL, about 150 mg/dL, about 160 mg/dL, about 170 mg/dL, about 180 mg/dL, about 190 mg/dL, about 200 mg/dL, or any value in between.

In some embodiments, the subject has an average Hb_{1AC} level of about 6% to about 10%, or more, for at least about 3 months (e.g., at least about 4 months, at least about 5 months, at least about 6 months, at least about 7 months, at least about 8 months, at least about 9 months, at least about 10 months, at least about 11 months, at least about a year, at least about 1.5 years, or at least about 2 years) prior to the first administration of atrasentan, or a pharmaceutically acceptable salt thereof, or prior to the first administration of atrasentan, or a pharmaceutically acceptable salt thereof, and a SGLT-2 inhibitor. For example, the subject has an average Hb_{1AC} level of about 6%, about 6.5%, about 7%, about 7.5%, about 8%, about 8.5%, about 9%, about 9.5%, 10%, or any value in between.

In some embodiments, the subject maintains a potassium level within the normal physiologic range during treatment with atrasentan, or a pharmaceutically acceptable salt thereof, and a SGLT-2 inhibitor. In certain embodiments, the subject maintains a potassium level within the normal physiologic range for at least about 3 months (e.g., at least about 4 months, at least about 5 months, at least about 6 months, at least about 7 months, at least about 8 months, at least about 9 months, at least about 10 months, at least about 11 months, at least about a year, at least about 1.5 years, or at least about 2 years) prior to the first administration of atrasentan, or a pharmaceutically acceptable salt thereof, and a SGLT-2 inhibitor. In certain embodiments, the subject maintains a potassium level within 3.5 to 5.2 mEq/L. For example, the subject maintains an average potassium level at about 3.5 mEq/L, about 3.6 mEq/L, about 3.7 mEq/L, about 3.8 mEq/L, about 3.9, about mEq/L, about 4.0 mEq/L, about 4.1 mEq/L, about 4.2 mEq/L, about 4.3 mEq/L, about 4.4 mEq/L, about 4.5 mEq/L, about 4.6 mEq/L, about 4.7 mEq/L, about 4.8 mEq/L, about 4.9 mEq/L, about 5.0 mEq/L, about 5.1 mEq/L, or about 5.2 mEq/L, or any value in between.

In some embodiments, the subject maintains a sodium level within the normal physiologic range. In certain embodiments, the subject maintains a sodium level within the normal physiologic range for at least about 3 months (e.g., at least about 4 months, at least about 5 months, at least about 6 months, at least about 7 months, at least about 8 months, at least about 9 months, at least about 10 months, at least about 11 months, at least about a year, at least about 1.5 years, or at least about 2 years) prior to the first administration of atrasentan, or a pharmaceutically acceptable salt thereof, and a SGLT-2 inhibitor. In certain embodiments, the subject maintains a sodium level within 135 to 145 mEq/L. For example, the subject maintains an average sodium level of about 135 mEq/L, about 136 mEq/L, about 137 mEq/L, about 138 mEq/L, about 139 mEq/L, about 140 mEq/L, about 141 mEq/L, about 142 mEq/L, about 143 mEq/L, about 144 mEq/L, about or 145 mEq/L, or any value in between.

In some embodiments, the subject has been receiving one or more inhibitors of the renin-angiotensin system for at least about four weeks prior to the first administration of atrasentan, or a pharmaceutically acceptable salt thereof, and a SGLT-2 inhibitor. For example, in some embodiments, the subject has been receiving one or more inhibitors of the renin-angiotensin system for at least about 4 weeks, about 10 weeks, about 20 weeks, about 40 weeks, or about 60 weeks, or any value in between, prior to the first administration of atrasentan, or a pharmaceutically acceptable salt thereof, and a SGLT-2 inhibitor.

In some embodiments, the subject has been receiving a maximally tolerated stable dose of the one or more renin-angiotensin system inhibitor. For example, the subject has been receiving a maximally tolerated stable dose of the one or more renin-angiotensin system inhibitor for at least about 4 weeks, about 10 weeks, about 14 weeks, about 16 weeks, about 18 weeks, about 20 weeks, about 25 weeks, about 30 weeks, about 35 weeks, about 40 weeks, about 45 weeks, or about 50 weeks, or any value in between, prior to the first administration of atrasentan, or a pharmaceutically acceptable salt thereof, and a SGLT-2 inhibitor. In some embodiments, the one or more inhibitors of the renin-angiotensin system is selected from the group consisting of diuretics, angiotensin converting enzyme (ACE) inhibitors, angiotensin II receptor blockers (ARBs), calcium channel blockers, renin inhibitors, and aldosterone antagonists. For example, the one or more inhibitors of the renin-angiotensin system can be ACE inhibitor, ARB, or a combination thereof, wherein the ACE inhibitor or ARB can be as described anywhere herein. For example, the ACE inhibitor can be selected from the group consisting of: quinapril, fosinopril perindopril, captopril, enalapril, enalaprilat, ramipril, cilazapril, delapril, fosenopril, zofenopril, indolapril, benazepril, lisinopril, spirapril, trandolapril, perindep, pentopril, moexipril, rescinnamine, and pivopril. For example, the ARB can be selected from the group consisting of: candesartan, candesartan cilexetil, eprosartan, irbesartan, losartan, olmesartan, olmesartan medoxomil, telmisartan, valsartan, azilsartan medoxomil, and BRA-657.

In some embodiments, the subject is receiving a diuretic In certain embodiments, the subject is receiving one or more diuretics, such as hydrochlorothiazide, trichlormethiazide, hydroflumethiazide, quinethazone, metolazone, chlorothiazide, chlorthalidone, indapamide, methyclothiazide bemetanide, torsemide, piretanide, ethacrynic acid, bumetanide, furosemide, triamterene, spironolactone, eplerenone, and amiloride.

In some embodiments, the subject is concomitantly (i.e., during treatment with atrasentan, or a pharmaceutically acceptable salt thereof, and a SGLT-2 inhibitor) receiving an inhibitor of one or more elements of the renin-angiotensin-aldosterone system. In certain embodiments, the subject is concomitantly receiving an ACE inhibitor, an ARB, and/or a diuretic. In some embodiments, a subject is concomitantly receiving an ACE inhibitor. In some embodiments, a subject is concomitantly receiving an ACE inhibitor and a diuretic. In some embodiments, a subject is concomitantly receiving an ARB. In some embodiments, a subject is concomitantly receiving an ARB and a diuretic. The ACE inhibitor can be, for example, one or more of quinapril, fosinopril perindopril, captopril, enalapril, enalaprilat, ramipril, cilazapril, delapril, fosenopril, zofenopril, indolapril, benazepril, lisinopril, spirapril, trandolapril, perindep, pentopril, moexipril, rescinnamine, and pivopril. The ARB can be, for example, one or more of candesartan, candesartan cilexetil, eprosartan, irbesartan, losartan, olmesartan, olmesartan medoxomil, telmisartan, valsartan, azilsartan medoxomil, and BRA-657. The diuretic can be, for example, one or more of hydrochlorothiazide, trichlormethiazide, hydroflumethiazide, quinethazone, metolazone, chlorothiazide, chlorthalidone, indapamide, methyclothiazide bemetanide, torsemide, piretanide, ethacrynic acid, bumetanide, furosemide, triamterene, spironolactone, eplerenone, and amiloride.

In some embodiments, a subject is receiving an additional therapeutic agent, such as a statin, a calcium channel blocker, a beta blocker, fish oil, or a combination of any of the foregoing. In certain embodiments, the subject is further concomitantly receiving one or more statins, such as atorvastatin, fluvastatin, lovastatin, pravastatin, rosuvastatin, simvastatin, and pitavastatin.

In some embodiments, the subject has not undergone organ transplantation prior to the first administration of atrasentan, or a pharmaceutically acceptable salt thereof, and a SGLT-2 inhibitor.

In some embodiments, the subject has not been diagnosed with heart failure prior to the first administration of atrasentan, or a pharmaceutically acceptable salt thereof, and a SGLT-2 inhibitor. In some embodiments, the subject has not been previously admitted to hospital for conditions relating to fluid overload prior to the first administration of atrasentan, or a pharmaceutically acceptable salt thereof, and a SGLT-2 inhibitor. Non-limiting examples of conditions relating to fluid overload include uncontrolled peripheral edema, facial edema, pleural effusion, or ascites.

In some embodiments, the subject has not been diagnosed with clinically significant liver disease prior to the first administration of atrasentan, or a pharmaceutically acceptable salt thereof, and a SGLT-2 inhibitor. In some embodiments, the transaminase or bilirubin values of the subject are no more than twice the normal upper limit prior to the first administration of atrasentan, or a pharmaceutically acceptable salt thereof, and a SGLT-2 inhibitor. For example, the ALT level of the subject is below about 110 U/L (e.g., below about 100 U/L, below 90 U/L, below about 80 U/L, below about 70 U/L, below about 60 U/L, below about 50 U/L, or below about 40 U/L, or any value in between). As another example, the AST level of the subject is below 100 U/L (e.g., below about 90 U/L, below about 80 U/L, below about 70 U/L, below about 60 U/L, below about 50 U/L, or below about 40 U/L, or any value in between). As yet another example, the bilirubin level of the subject is below about 2.5 mg/dL (e.g., below about 2 mg/dL, below about 1.5 mg/dL, below about 1.4 mg/dL, below about 1.3 mg/dL, below about 1.2 mg/dL, below about 1.1 mg/dL, below about 1.0 mg/dL, or below about 0.9 mg/dL, or any value in between). In some embodiments, the ALT level of the subject is about 30 U/L to about 110 U/L, for example, about 30 U/L to about 80 U/L, about 50 U/L to about 90 U/L, about 70 U/L to about 110 U/L, about 30 U/L to about 50 U/L, about 40 U/L to about 60 U/L, about 50 U/L to about 70 U/L, about 60 U/L to about 80 U/L, about 70 U/L to about 90 U/L, about 80 U/L to about 100 U/L, about 100 U/L to about 110 U/L, or any value in between.

In some embodiments, the subject has a hemoglobin level of above about 9 g/dL (e.g., above about 10 g/dL, about 11 g/dL, about 12 g/dL, or about 13 g/dL, or any value in between) prior to the first administration of atrasentan, or a pharmaceutically acceptable salt thereof, and a SGLT-2 inhibitor. In some embodiments, the subject has not received blood transfusion for anemia for at least about 3 months (e.g., at least about 4 months, about 5 months, about 6 months, or about one year) prior to the first administration of atrasentan, or a pharmaceutically acceptable salt thereof, and a SGLT-2 inhibitor. In some embodiments, the subject has a hemoglobin level of about 9 g/dL to about 13 g/dL, or any value in between, prior to the first administration of atrasentan, or a pharmaceutically acceptable salt thereof, and a SGLT-2 inhibitor. For example, about 10 g/dL, about 11 g/dL, about 12 g/dL, or about 13 g/dL, or any value in between.

In some embodiments, the subject has not been diagnosed with cancer for at least 5 years prior to the first administration of atrasentan, or a pharmaceutically acceptable salt thereof, and a SGLT-2 inhibitor. In some embodiments, the subject has not been diagnosed with cancer (e.g., lung cancer or prostate cancer) for at least 5 years prior to the first administration of atrasentan, or a pharmaceutically acceptable salt thereof, and a SGLT-2 inhibitor. In some embodiments, the subject has not been diagnosed with cancer for at least 5 years prior to the first administration of atrasentan, or a pharmaceutically acceptable salt thereof, and a SGLT-2 inhibitor, unless the cancer is nonmelanoma skin cancer not requiring ongoing treatment. In some embodiments, the subject does not have cancer prior to the first administration of atrasentan, or a pharmaceutically acceptable salt thereof, and a SGLT-2 inhibitor, unless the cancer is non-melanoma skin cancer not requiring ongoing treatment. In some embodiments, the subject does not suffer from cancer prior to the first administration of atrasentan, or a pharmaceutically acceptable salt thereof, and a SGLT-2 inhibitor, unless the cancer is non-melanoma skin cancer not requiring ongoing treatment. In some embodiments, the subject is not being treated for cancer for at least 5 years prior to the first administration of atrasentan, or a pharmaceutically acceptable salt thereof, and a SGLT-2 inhibitor unless the cancer is non-melanoma skin cancer not requiring ongoing treatment.

In some embodiments, the subject has not been previously diagnosed with one or more of IgA nephropathy, HIV/AIDS, or acute kidney failure. In some embodiments, the subject has not been previously diagnosed with one or more of IgA nephropathy, HIV/AIDS, cancer (e.g., prostate cancer or lung cancer), or acute kidney failure. In some embodiments, the subject has not been previously diagnosed with one or more of IgA nephropathy, HIV/AIDS, prostate cancer, or acute kidney failure. In some embodiments, the subject has not been previously diagnosed with one or more of IgA nephropathy, HIV-related nephropathy, prostate cancer, or acute kidney failure. In some embodiments, the subject has not been previously diagnosed with one or more of IgA nephropathy, HIV-related nephropathy, cancer (e.g., lung cancer, or prostate cancer), or acute kidney failure. In some embodiments, the subject has not been previously diagnosed with one or more of IgA nephropathy, HIV-related nephropathy, or acute kidney failure. In certain embodiments, the subject has not been previously diagnosed with IgA nephropathy. In certain embodiments, the subject has not been previously diagnosed with HIV/AIDS. In certain embodiments, the subject has not been previously diagnosed with acute kidney failure. In certain embodiments, the subject has not been previously diagnosed with HIV-related nephropathy. In certain embodiments, the subject has not been diagnosed with cancer. In certain embodiments, the subject has not been diagnosed with prostate cancer. In certain embodiments, the subject has not been diagnosed with lung cancer. In certain embodiments, the subject has not been previously diagnosed with any one of IgA nephropathy, HIV/AIDS, and acute kidney failure. In certain embodiments, the subject has not been previously diagnosed with any one of IgA nephropathy, HIV/AIDS, prostate cancer, and acute kidney failure. In certain embodiments, the subject has not been previously diagnosed with any one of IgA nephropathy, HIV-related nephropathy, prostate cancer, and acute kidney failure. In certain embodiments, the subject has not been previously diagnosed with any one of IgA nephropathy, HIV-related nephropathy, and acute kidney failure.

In some embodiments, the subject does not have one or more of IgA nephropathy, HIV/AIDS, or acute kidney failure. In some embodiments, the subject does not have one or more of IgA nephropathy, HIV/AIDS, prostate cancer, or acute kidney failure. In some embodiments, the subject does not have one or more of IgA nephropathy, HIV-related nephropathy, prostate cancer, or acute kidney failure. In some embodiments, the subject does not have one or more of IgA nephropathy, HIV-related nephropathy, or acute kidney failure. In certain embodiments, the subject does not have IgA nephropathy. In certain embodiments, the subject does not have HIV/AIDS. In certain embodiments, the subject does not have acute kidney failure. In certain embodiments, the subject does not have HIV-related nephropathy. In certain embodiments, the subject does not have prostate cancer. In certain embodiments, the subject does not have any one of IgA nephropathy, HIV/AIDS, and acute kidney failure. In certain embodiments, the subject does not have any one of IgA nephropathy, HIV/AIDS, prostate cancer, and acute kidney failure. In certain embodiments, the subject does not have any one of IgA nephropathy, HIV-related nephropathy, prostate cancer, and acute kidney failure. In certain embodiments, the subject does not have any one of IgA nephropathy, HIV-related nephropathy, and acute kidney failure.

In some embodiments, the subject does not suffer from one or more of IgA nephropathy, HIV/AIDS, or acute kidney failure. In some embodiments, the subject does not suffer from one or more of IgA nephropathy, HIV/AIDS, prostate cancer, or acute kidney failure. In some embodiments, the subject does not suffer from one or more of IgA nephropathy, HIV-related nephropathy, prostate cancer, or acute kidney failure. In some embodiments, the subject does not suffer from one or more of IgA nephropathy, HIV-related nephropathy, or acute kidney failure. In certain embodiments, the subject does not suffer from IgA nephropathy. In certain embodiments, the subject does not suffer from HIV/AIDS. In certain embodiments, the subject does not suffer from acute kidney failure. In certain embodiments, the subject does not suffer from HIV-related nephropathy. In certain embodiments, the subject does not suffer from prostate cancer. In certain embodiments, the subject does not suffer from any one of IgA nephropathy, HIV/AIDS, and acute kidney failure. In certain embodiments, the subject does not suffer from any one of IgA nephropathy, HIV/AIDS, prostate cancer, and acute kidney failure. In certain embodiments, the subject does not suffer from any one of IgA nephropathy, HIV-related nephropathy, prostate cancer, and acute kidney failure. In certain embodiments, the subject does not suffer from any one of IgA nephropathy, HIV-related nephropathy, and acute kidney failure.

In some embodiments, the subject is not being treated for one or more of IgA nephropathy, HIV/AIDS, or acute kidney failure. In some embodiments, the subject is not being treated for one or more of IgA nephropathy, HIV/AIDS, prostate cancer, or acute kidney failure. In some embodiments, the subject is not being treated for one or more of IgA nephropathy, HIV-related nephropathy, prostate cancer, or acute kidney failure. In some embodiments, the subject is not being treated for one or more of IgA nephropathy, HIV-related nephropathy, or acute kidney failure. In certain embodiments, the subject is not being treated for IgA nephropathy. In certain embodiments, the subject is not being treated for HIV/AIDS. In certain embodiments, the subject is not being treated for acute kidney failure. In certain embodiments, the subject is not being treated for HIV-related nephropathy. In certain embodiments, the subject is not being treated for prostate cancer. In certain embodiments, the subject is not being treated for any one of IgA nephropathy, HIV/AIDS, and acute kidney failure. In certain embodiments, the subject is not being treated for any one of IgA nephropathy, HIV/AIDS, prostate cancer, and acute kidney failure. In certain embodiments, the subject is not being treated for any one of IgA nephropathy, HIV-related nephropathy, prostate cancer, and acute kidney failure. In certain embodiments, the subject is not being treated for any one of IgA nephropathy, HIV-related nephropathy, and acute kidney failure.

In some embodiments, the subject has been determined to have controlled serum glucose levels; or the subject has not been diagnosed with one or more of HIV-related nephropathy or acute kidney failure. In certain embodiments, the subject has been determined to have controlled serum glucose levels. For example, the subject has been determined to have a fasting serum glucose level of below about 130 mg/dL, below about 125 mg/dL, below about 120 mg/dL, below about 115 mg/dL, below about 110 mg/dL, below about 105 mg/dL, below about 100 mg/dL, below about 95 mg/dL, below about 90 mg/dL, below about 85 mg/dL, below about 80 mg/dL, or below about 75 mg/dL, or any value in between. In certain embodiments, the subject has not been diagnosed with one or more of HIV-related nephropathy or acute kidney failure. In certain embodiments, the subject has been determined to have controlled serum glucose levels as described anywhere herein; and the subject has not been diagnosed with one or more of HIV-related nephropathy or acute kidney failure.

In some embodiments, the subject has not been previously diagnosed with a chronic kidney disease that is other than DKD. Non-limiting examples include a hypertensive kidney disease, or a primary glomerulopathy that is determined to not be associated with DKD. In certain embodiments, the subject has not been previously diagnosed with a hypertensive kidney disease. In certain embodiments, the subject has not been diagnosed with a primary glomerulopathy that is determined to not be associated with IgA nephropathy.

In some embodiments, the subject does not have a chronic kidney disease that is other than DKD. Non-limiting examples include a hypertensive kidney disease, or a primary glomerulopathy that is determined to not be associated with DKD. In certain embodiments, the subject does not have a hypertensive kidney disease. In certain embodiments, the subject does not have a primary glomerulopathy that is determined to not be associated with DKD.

In some embodiments, the subject does not suffer from a chronic kidney disease that is other than DKD. Non-limiting examples include a hypertensive kidney disease, or a primary glomerulopathy that is determined to not be associated with DKD. In certain embodiments, the subject does not suffer from a hypertensive kidney disease. In certain embodiments, the subject does not suffer from a primary glomerulopathy that is determined to not be associated with DKD.

In some embodiments, the subject is not being treated for a chronic kidney disease that is other than DKD. Non-limiting examples include a hypertensive kidney disease, or a primary glomerulopathy that is determined to not be associated with DKD. In certain embodiments, the subject is not being treated for a hypertensive kidney disease. In certain embodiments, the subject is not being treated for a primary glomerulopathy that is determined to not be associated with DKD.

In some embodiments, the UACR of the subject is decreased after treatment with atrasentan, or a pharmaceutically acceptable salt thereof, and a SGLT-2 inhibitor, relative to a subject not administered atrasentan, or a pharmaceutically acceptable salt thereof, and the SGLT-2 inhibitor. In some embodiments, the UACR of the subject is decreased by at least about 10% after treatment with atrasentan, or a pharmaceutically acceptable salt thereof, and a SGLT-2 inhibitor, relative to a subject not administered atrasentan, or a pharmaceutically acceptable salt thereof, and the SGLT-2 inhibitor (e.g., after treatment for about 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between). In some embodiments, the UACR of the subject is decreased by at least about 20%, by at least about 30%, by at least about 40%, by at least about 50%, by at least about 60%, by at least about 70%, by at least about 80%, by at least about 90%, or by at least about 95%, or any value in between, relative to a subject not administered atrasentan, or a pharmaceutically acceptable salt thereof, and the SGLT-2 inhibitor. In certain of the foregoing embodiments, the subject has been treated with atrasentan, or a pharmaceutically acceptable salt thereof, and a SGLT-2 inhibitor for about 15 days to about 30 days. In certain of the foregoing embodiments, the subject has been treated with atrasentan, or a pharmaceutically acceptable salt thereof, and a SGLT-2 inhibitor for about 1 month to about 12 months.

In some embodiments, fluid retention or body weight of the subject is decreased after treatment with atrasentan, or a pharmaceutically acceptable salt thereof, and a SGLT-2 inhibitor, relative to a subject not administered atrasentan, or a pharmaceutically acceptable salt thereof, and the SGLT-2 inhibitor. In some embodiments, fluid retention or body weight of the subject is decreased by at least about 10% after treatment with atrasentan, or a pharmaceutically acceptable salt thereof, and a SGLT-2 inhibitor, relative to a subject not administered atrasentan, or a pharmaceutically acceptable salt thereof, and the SGLT-2 inhibitor (e.g., after treatment for about 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between). In some embodiments, fluid retention or body weight of the subject is decreased by at least about 1 kg, by at least about 2 kg, by at least about 3 kg, by at least about 4 kg, by at least about 5 kg, by at least about 6 kg, by at least about 7 kg, by at least about 8 kg, by at least about 9 kg or by at least about 10 kg, or any value in between, relative to a subject not administered atrasentan, or a pharmaceutically acceptable salt thereof, and the SGLT-2 inhibitor. In certain of the foregoing embodiments, the subject has been treated with atrasentan, or a pharmaceutically acceptable salt thereof, and a SGLT-2 inhibitor for about 15 days to about 30 days. In certain of the foregoing embodiments, the subject has been treated with atrasentan, or a pharmaceutically acceptable salt thereof, and a SGLT-2 inhibitor for about 1 month to about 12 months.

In some embodiments, the BNP level in the subject is decreased after treatment with atrasentan, or a pharmaceutically acceptable salt thereof, and a SGLT-2 inhibitor, relative to a subject not administered atrasentan, or a pharmaceutically acceptable salt thereof, and the SGLT-2 inhibitor. In some embodiments, the BNP level of the subject is decreased by at least about 10% after treatment with atrasentan, or a pharmaceutically acceptable salt thereof, and a SGLT-2 inhibitor, relative to a subject not administered atrasentan, or a pharmaceutically acceptable salt thereof, and the SGLT-2 inhibitor (e.g., after treatment for 1 about week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between). In some embodiments, the BNP level of the subject is decreased by at least about 20%, by at least about 30%, by at least about 40%, by at least about 50%, by at least about 60%, by at least about 70%, by at least about 80%, by at least about 90%, or by at least about 95%, or any value in between, relative to a subject not administered atrasentan, or a pharmaceutically acceptable salt thereof, and the SGLT-2 inhibitor. In certain of the foregoing embodiments, the subject has been treated with atrasentan, or a pharmaceutically acceptable salt thereof, and a SGLT-2 inhibitor for about 15 days to about 30 days. In certain of the foregoing embodiments, the subject has been treated with atrasentan, or a pharmaceutically acceptable salt thereof, and a SGLT-2 inhibitor for about 1 month to about 12 months.

In some embodiments, the rate of decrease of eGFR of the subject is reduced by at least about 10% after treatment with atrasentan, or a pharmaceutically acceptable salt thereof, and a SGLT-2 inhibitor, relative to a subject not administered atrasentan, or a pharmaceutically acceptable salt thereof, and the SGLT-2 inhibitor (e.g., after treatment for about 1 week, 2 weeks, 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between). In certain embodiments, the rate of decrease of eGFR of the subject is reduced by at least about 20%, relative to a subject not administered atrasentan, or a pharmaceutically acceptable salt thereof, and the SGLT-2 inhibitor. For example, in some embodiments, the rate of decrease of eGFR of the subject is reduced by at least about 30%, by at least about 40%, by at least about 50%, by at least about 60% by at least about 70%, by at least about 80%, by at least about 90%, or by at least about 95%, or any value in between. In certain of the foregoing embodiments, the subject has been treated with atrasentan, or a pharmaceutically acceptable salt thereof, and a SGLT-2 inhibitor for about 15 days to about 30 days. In certain of the foregoing embodiments, the subject has been treated with atrasentan, or a pharmaceutically acceptable salt thereof, and a SGLT-2 inhibitor for about 6 months to about 1 year.

In some embodiments, the rate of decrease of eGFR of the subject is reduced to below about 10 mL/min per year after treatment with atrasentan, or a pharmaceutically acceptable salt thereof, and a SGLT-2 inhibitor (e.g., after treatment for about 1 week, about 2 weeks, about 3 weeks, about 4 weeks, about 5 weeks, about 6 weeks, about 7 weeks, about 8 weeks, about 9 weeks, about 10 weeks, about 20 weeks, about 30 weeks, about 40 weeks, about 50 weeks, about 60 weeks, about 70 weeks, about 80 weeks, about 90 weeks, about 100 weeks, about 110 weeks, about 120 weeks, about 130 weeks, about 140 weeks, about 150 weeks, about 160 weeks, about 170 weeks, about 180 weeks, about 190 weeks, or about 200 weeks, or any value in between). In some embodiments, the rate of decrease of eGFR of the subject is reduced to below about 9 mL/min per year. For example, in some embodiments, the rate of decrease of eGFR of the subject is reduced to below about 8 mL/min per year, to below about 7 mL/min per year, to below about 6 mL/min per year, to below about 5 mL/min per year, to below about 4 mL/min per year, to below about 3 mL/min per year, to below about 2 mL/min per year, or to below about 1 mL/min per year, or any value in between. In certain of the foregoing embodiments, the subject has been treated with atrasentan, or a pharmaceutically acceptable salt thereof, and a SGLT-2 inhibitor for about 15 days to about 30 days. In certain of the foregoing embodiments, the subject has been treated with atrasentan, or a pharmaceutically acceptable salt thereof, and a SGLT-2 inhibitor for about 6 months to about 1 year. In some embodiments, the rate of decrease of eGFR of the subject is reduced to about 9 mL/min per year to about 1 mL/min per year, for example, about 8 mL/min per year, about 7 mL/min per year, about 6 mL/min per year, about 5 mL/min per year, about 4 mL/min per year, about 3 mL/min per year, about 2 mL/min per year, about 1 mL/min per year, or any value in between.

In some embodiments, the UACR of the subject is decreased by about 10% to about 95%; and the fluid retention of the subject is decreased by about 10% to about 95%; after treatment with atrasentan, or a pharmaceutically acceptable salt thereof, and a SGLT-2 inhibitor, relative to a subject not administered atrasentan, or a pharmaceutically acceptable salt thereof, and the SGLT-2 inhibitor. In some embodiments, the UACR of the subject is decreased by about 10% to about 40%, by about 20% to about 50%, by about 30% to about 60%, by about 50% to about 80%, or by about 65% to about 95%; and the fluid retention of the subject is decreased by about 10% to about 30%, or by about 20% to about 40%; after treatment with atrasentan, or a pharmaceutically acceptable salt thereof, and a SGLT-2 inhibitor, relative to a subject not administered atrasentan, or a pharmaceutically acceptable salt thereof, and the SGLT-2 inhibitor.

In some embodiments, the UACR of the subject is decreased by about 10% to about 95%; and the BNP level of the subject is decreased by about 10% to about 95%; after treatment with atrasentan, or a pharmaceutically acceptable salt thereof, and a SGLT-2 inhibitor, relative to a subject not administered atrasentan, or a pharmaceutically acceptable salt thereof, and the SGLT-2 inhibitor. In some embodiments, the UACR of the subject is decreased by about 10% to about 40%, by about 20% to about 50%, by about 30% to about 60%, by about 50% to about 80%, or by about 65% to about 95%; and the BNP level of the subject is decreased by about 10% to about 40%, by about 30% to about 60%, by about 50% to about 80%, or by about 65% to about 95%; after treatment with atrasentan, or a pharmaceutically acceptable salt thereof, and a SGLT-2 inhibitor, relative to a subject not administered atrasentan, or a pharmaceutically acceptable salt thereof, and the SGLT-2 inhibitor.

In some embodiments, the UACR of the subject is decreased by about 10% to about 95%; and the rate of decrease of eGFR of the subject is reduced by about 10% to about 95%; after treatment with atrasentan, or a pharmaceutically acceptable salt thereof, and a SGLT-2 inhibitor, relative to a subject not administered atrasentan, or a pharmaceutically acceptable salt thereof, and the SGLT-2 inhibitor. In some embodiments, the UACR of the subject is decreased by about 10% to about 40%, by about 20% to about 50%, by about 30% to about 60%, by about 50% to about 80%, or by about 65% to about 95%; and the rate of decrease of eGFR of the subject is reduced by about 10% to about 40%, by about 30% to about 60%, by about 50% to about 80%, or by about 65% to about 95%; after treatment with atrasentan, or a pharmaceutically acceptable salt thereof, and a SGLT-2 inhibitor, relative to a subject not administered atrasentan, or a pharmaceutically acceptable salt thereof, and the SGLT-2 inhibitor.

In some embodiments, the UACR of the subject is decreased by about 10% to about 95%; and the BNP level of the subject is decreased by about 10% to about 95%; and the rate of decrease of eGFR of the subject is reduced by about 10% to about 95%; after treatment with atrasentan, or a pharmaceutically acceptable salt thereof, and a SGLT-2 inhibitor, relative to a subject not administered atrasentan, or a pharmaceutically acceptable salt thereof, and the SGLT-2 inhibitor. In some embodiments, the UACR of the subject is decreased by about 10% to about 40%, by about 20% to about 50%, by about 30% to about 60%, by about 50% to about 80%, or by about 65% to about 95%; and the BNP level of the subject is decreased by about 10% to about 95%; and the rate of decrease of eGFR of the subject is reduced by about 10% to about 40%, by about 30% to about 60%, by about 50% to about 80%, or by about 65% to about 95%; after treatment with atrasentan, or a pharmaceutically acceptable salt thereof, and a SGLT-2 inhibitor, relative to a subject not administered atrasentan, or a pharmaceutically acceptable salt thereof, and the SGLT-2 inhibitor.

In some embodiments, the fluid retention of the subject is decreased by about 10% to about 95%; and the BNP level of the subject is decreased by about 10% to about 95%; after treatment with atrasentan, or a pharmaceutically acceptable salt thereof, and a SGLT-2 inhibitor, relative to a subject not administered atrasentan, or a pharmaceutically acceptable salt thereof, and the SGLT-2 inhibitor. In some embodiments, the fluid retention of the subject is decreased by about 10% to about 40%, by about 30% to about 60%, by about 50% to about 80%, or by about 65% to about 95%; and the BNP level of the subject is decreased by about 10% to about 40%, by about 30% to about 60%, by about 50% to about 80%, or by about 65% to about 95%; after treatment with atrasentan, or a pharmaceutically acceptable salt thereof, and a SGLT-2 inhibitor, relative to a subject not administered atrasentan, or a pharmaceutically acceptable salt thereof, and the SGLT-2 inhibitor.

In some embodiments, the fluid retention of the subject is decreased by about 10% to about 95%; and the rate of decrease of eGFR of the subject is reduced by about 10% to about 95%; after treatment with atrasentan, or a pharmaceutically acceptable salt thereof, and a SGLT-2 inhibitor, relative to a subject not administered atrasentan, or a pharmaceutically acceptable salt thereof, and the SGLT-2 inhibitor. In some embodiments, the fluid retention of the subject is decreased by about 10% to about 40%, by about 30% to about 60%, by about 50% to about 80%, or by about 65% to about 95%; and the rate of decrease of eGFR of the subject is reduced by about 10% to about 40%, by about 30% to about 60%, by about 50% to about 80%, or by about 65% to about 95%; after treatment with atrasentan, or a pharmaceutically acceptable salt thereof, and a SGLT-2 inhibitor, relative to a subject not administered atrasentan, or a pharmaceutically acceptable salt thereof, and the SGLT-2 inhibitor.

In some embodiments, the fluid retention of the subject is decreased by about 10% to about 95%; the BNP level of the subject is decreased by about 10% to about 95%; and the rate of decrease of eGFR of the subject is reduced by about 10% to about 95%; after treatment with atrasentan, or a pharmaceutically acceptable salt thereof, and a SGLT-2 inhibitor, relative to a subject not administered atrasentan, or a pharmaceutically acceptable salt thereof, and the SGLT-2 inhibitor. In some embodiments, the fluid retention of the subject is decreased by about 10% to about 40%, by about 30% to about 60%, by about 50% to about 80%, or by about 65% to about 95%; the BNP level of the subject is decreased by about 10% to about 40%, by about 30% to about 60%, by about 50% to about 80%, or by about 65% to about 95%; and the rate of decrease of eGFR of the subject is reduced by about 10% to about 95%; after treatment with atrasentan, or a pharmaceutically acceptable salt thereof, and a SGLT-2 inhibitor, relative to a subject not administered atrasentan, or a pharmaceutically acceptable salt thereof, and the SGLT-2 inhibitor.

In some embodiments, the BNP level of the subject is decreased by about 10% to about 95%; and the rate of decrease of eGFR of the subject is reduced by about 10% to about 95%; after treatment with atrasentan, or a pharmaceutically acceptable salt thereof, and a SGLT-2 inhibitor, relative to a subject not administered atrasentan, or a pharmaceutically acceptable salt thereof, and the SGLT-2 inhibitor. In some embodiments, the BNP level of the subject is decreased by about 10% to about 40%, by about 30% to about 60%, by about 50% to about 80%, or by about 65% to about 95%; and the rate of decrease of eGFR of the subject is reduced by about 10% to about 40%, by about 30% to about 60%, by about 50% to about 80%, or by about 65% to about 95%; after treatment with atrasentan, or a pharmaceutically acceptable salt thereof, and a SGLT-2 inhibitor, relative to a subject not administered atrasentan, or a pharmaceutically acceptable salt thereof, and the SGLT-2 inhibitor.

In some embodiments, the subject is about 15 to about 90 years old. In some embodiments, the subject is about 15 to about 40 years old. In some embodiments, the subject is about 18 to about 90 years old. In some embodiments, the subject is about 18 to about 40 years old. In some embodiments, the subject is about 35 to about 85 years old.

Some embodiments provide a method of treating DKD in a subject in need thereof, comprising: a) determining that the subject has diabetes and associated nephropathy; and b) administering a therapeutically effective amount of atrasentan, or a pharmaceutically acceptable salt thereof, and a SGLT-2 inhibitor to the subject. In some embodiments, the subject has not been previously diagnosed with one or more of IgA nephropathy, HIV/AIDS, or acute kidney failure. In some embodiments, the subject has not been previously diagnosed with HIV-related nephropathy. In some embodiments, the subject has not been previously diagnosed with cancer. In some embodiments, the cancer is lung cancer or prostate cancer.

In some embodiments, the subject has been administered a therapeutically effective dose of a RAS inhibitor for at least four weeks, at least 10 weeks, or at least 12 weeks, prior to administration of a therapeutically effective amount of atrasentan, or a pharmaceutically acceptable salt thereof, and a SGLT-2 inhibitor. In some embodiments, the subject is concurrently administered a therapeutically effective dose of a RAS inhibitor and a therapeutically effective amount of atrasentan, or a pharmaceutically acceptable salt thereof, and a SGLT-2 inhibitor. In some embodiments, the RAS inhibitor is an angiotensin-converting enzyme (ACE) inhibitor. In some embodiments, the RAS inhibitor is an angiotensin receptor blocker (ARB). In some embodiments, the subject is further administered a diuretic.

In some embodiments, the subject has been administered a maximally tolerated stable dose of a RAS inhibitor for at least 4 weeks, at least 10 weeks, or at least 12 weeks prior to administration of a therapeutically effective amount of atrasentan, or a pharmaceutically acceptable salt thereof, and an SGLT-2 inhibitor. In some embodiments, the subject is concurrently administered a maximally tolerated stable dose of a RAS inhibitor and a therapeutically effective amount of atrasentan, or a pharmaceutically acceptable salt thereof, and a SGLT-2 inhibitor. In some embodiments, the RAS inhibitor is an angiotensin-converting (ACE) enzyme inhibitor. In some embodiments, the RAS inhibitor is an angiotensin receptor blocker (ARB). In some embodiments, the subject is further administered a diuretic.

In some embodiments, the subject has been administered a maximally tolerated stable dose of an ACE inhibitor or an ARB for at least 4 weeks prior to the first administration of atrasentan, or a pharmaceutically acceptable salt thereof. In some embodiments, the subject has been administered a maximally tolerated stable dose of an ACE inhibitor or an ARB for at least 10 weeks prior to the first administration of atrasentan, or a pharmaceutically acceptable salt thereof. In some embodiments, the subject has been administered a maximally tolerated stable dose of an ACE inhibitor or an ARB for at least 12 weeks prior to the first administration of atrasentan, or a pharmaceutically acceptable salt thereof.

In some embodiments, the subject has been administered a maximally tolerated stable dose of an ACE inhibitor or an ARB, and a diuretic, for at least 4 weeks prior to the first administration of atrasentan, or a pharmaceutically acceptable salt thereof. In some embodiments, the subject has been administered a maximally tolerated stable dose of an ACE inhibitor or an ARB, and a diuretic, for at least 10 weeks prior to the first administration of atrasentan, or a pharmaceutically acceptable salt thereof. In some embodiments, the subject has been administered a maximally tolerated stable dose of an ACE inhibitor or an ARB, and a diuretic, for at least 12 weeks prior to the first administration of atrasentan, or a pharmaceutically acceptable salt thereof.

In any of the foregoing embodiments, a subject that is “not administered atrasentan, or a pharmaceutically acceptable salt thereof, and the SGLT-2 inhibitor” refers to a subject that is administered, for example, a placebo, atrasentan (or a pharmaceutically acceptable salt thereof) as a monotherapy, a SGLT-2 inhibitor as a monotherapy, or some other therapeutic agent(s).

### D. Atrasentan

Atrasentan, also known as (2R,3R,4S)-4-(1,3-benzodioxol-5-yl)-1-[2-(dibutylamino)-2-oxoethyl]-2-(4-methoxyphenyl)pyrrolidine-3-carboxylic acid, ABT-627, A-147627, or A-127722, is a small molecule of the following chemical structure:

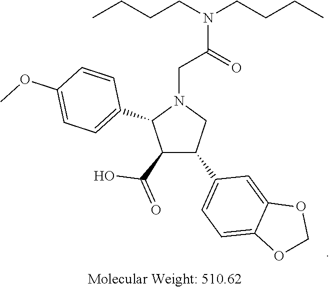 [SMILES: CCCCN(CCCC)C(=O)CN1C[C@H](c2ccc3c(c2)OCO3)[C@@H](C(=O)O)[C@@H]1c1ccc(OC)cc1]

Atrasentan and methods of preparation thereof are described in U.S. Pat. No. 7,208,517 and International Patent Application Publication No. WO 1997/030045 (see, e.g., Example 501), each of which is incorporated herein by reference in its entirety.

In some embodiments, atrasentan is administered as a free base. In some other embodiments, atrasentan is administered as a pharmaceutically acceptable salt as described anywhere herein.

Atrasentan is an ETA inhibitor which is about 1,860 times more selective for ETA relative to ETB. As used herein “ETA” is the abbreviation for endothelin receptor A; and “ETB” is the abbreviation of endothelin receptor B. See, e.g., Ann Rheum Dis., 66(11), pp. 1467-1472 (2007); Eur. Resp. J., 37, pp. 475-476 (2011); Plos One, 9, e87548 (2014); J. Clin. Oncol., 10, 31(14), pp. 1740-7 (2013); Pharmacol. Rev., 68 (2) pp. 357-418 (2016); and Nephrol. Dial. Transplant., 29, pp. i69-i73 (2014).

### Salts

In some embodiments, atrasentan is in the form of a pharmaceutically acceptable salt. The phrase “pharmaceutically acceptable salt” as used herein, refers to pharmaceutically acceptable organic or inorganic salts of a compound of the disclosure (e.g., atrasentan), Exemplary salts include acid addition salts formed by the reaction between atrasentan and an acid (e.g., organic acid or inorganic acid). Non-limiting examples include: sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, mandelate (e.g., (S)-mandelate or (R)-mandelate), gentisinate, fumarate, gluconate, glucuronate, saccharate, formate, benzoate, glutamate, methanesulfonate “mesylate”, ethanesulfonate, benzenesulfonate, and p-toluenesulfonate, pamoate (i.e., 4′-methylene-bis-(2-hydroxy-3-naphthoate)) salts. Exemplary salts also include base addition salts formed by the reaction between atrasentan and a base. Non-limiting examples include: alkali metal (e.g., sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ion. When referring to atrasentan, the term “salt” or “salts” is understood to be a salt of atrasentan that can be present alone or in a mixture with free atrasentan.

In some embodiments, atrasentan is in the form of a hydrochloride salt. The hydrochloride salt of atrasentan, also known as atrasentan hydrochloride (CAS Number: 195733-43-8); atrasentan hydrogen chloride; atrasentan hydrochloride salt; atrasentan chloride salt; atrasentan HCl; atrasentan monohydrochloride; (2R,3R,4S)-4-(1,3-benzodioxol-5-yl)-1-[2-(dibutylamino)-2-oxoethyl]-2-(4-methoxyphenyl) pyrrolidine-3-carboxylic acid, monohydrochloride; 3-pyrrolidinecarboxylic acid, 4-(1,3-benzodioxol-5-yl)-1-[2-(dibutylamino)-2-oxoethyl]-2-(4-methoxyphenyl)-, hydrochloride (1:1), (2R,3R, 4S)—; (2R,3R, 4S)-1-[2-dibutylcarbamoyl)methyl]-2-(p-methoxyphenyl)-4-[3,4-(methylenedioxy)phenyl]-3-pyrrolidinecarboxylic acid, monohydrochloride; 3-pyrrolidinecarboxylic acid, 4-(1,3-benzodioxol-5-yl)-1-[2-(dibutylamino)-2-oxoethyl]-2-(4-methoxyphenyl)-, monohydrochloride, [2R-(2α, 3β, 4α)]; ABT-627; A-147627.1; Abbott-147627.1, has the following structure:

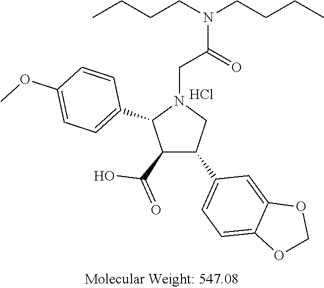 [SMILES: CCCCN(CCCC)C(=O)CN1C[C@H](c2ccc3c(c2)OCO3)[C@@H](C(=O)O)[C@@H]1c1ccc(OC)cc1 ; Cl]

wherein the molar ratio of atrasentan to chloride is 1:1. Atrasentan hydrochloride and methods of preparation thereof are further described in U.S. Pat. No. 7,208,517 and International Patent Application Publication No. WO 1997/030045 (see, e.g., Example 501), each of which is incorporated herein by reference in its entirety.

In some embodiments, atrasentan is in the form of a mandelate salt. In certain embodiments, atrasentan is in the form of a (S)-mandelate salt. In certain embodiments, atrasentan is in the form of a (R)-mandelate salt. In certain embodiments, in the atrasentan mandelate salt, atrasentan and mandelate has a molar ratio of 1:1. In certain embodiments, in the atrasentan mandelate salt, atrasentan and mandelate has a molar ratio of 2:1. Atrasentan mandelate salt and methods of preparation thereof are further described in U.S. Pat. Nos. 8,962,675 and 9,637,476, each of which is incorporated herein by reference in its entirety.

In some embodiments, atrasentan is in the form of a hemisulfate salt. Hemisulfate salt and methods of preparation thereof are further described in U.S. Pat. Nos. 8,962,675 and 9,637,476, each of which is incorporated herein by reference in its entirety.

In some embodiments, the atrasentan, or a pharmaceutically acceptable salt thereof is in the form of an anhydrate. In certain embodiments, the atrasentan, or a pharmaceutically acceptable salt thereof is in the form of a hydrate. In certain embodiments, the atrasentan, or a pharmaceutically acceptable salt thereof is in the form of a solvate.

### Stereochemistry

Atrasentan possesses three asymmetric centers and can be produced as individual stereoisomers (e.g., enantiomers or diastereomers) or as mixtures thereof as described in U.S. Pat. No. 7,208,517 and International Patent Application Publication No. WO 1997/030045. In some embodiments, atrasentan as described herein comprises the (2R,3R,4S)-stereoisomer, that is (2R,3R,4S)-4-(1,3-benzodioxol-5-yl)-1-[2-(dibutylamino)-2-oxoethyl]-2-(4-methoxyphenyl)pyrrolidine-3-carboxylic acid. In certain embodiments, atrasentan is the (2R,3R,4S)-stereoisomer that is substantially free of the other stereoisomers (e.g., contains <10%, <5%, <2%, <1%, <0.5%, <0.1%, or <0.05% of other stereoisomers).

### Polymorphs

Atrasentan, or a pharmaceutically acceptable salt thereof, as described herein, can be in one or more polymorphic forms. In some embodiments, atrasentan, or a pharmaceutically acceptable salt thereof is substantially amorphous (e.g., >75%, >80%, >85%, >90%, >95%, >98%, >99%, or >99.5% amorphous). In some embodiments, atrasentan, or a pharmaceutically acceptable salt thereof is substantially crystalline (e.g., >75%, >80%, >85%, >90%, >95%, >98%, >99%, or >99.5% crystalline).

In certain embodiments, the atrasentan, or a pharmaceutically acceptable salt thereof comprises Atrasentan Hydrochloride Crystalline Form 1. In certain embodiments, the atrasentan, or a pharmaceutically acceptable salt thereof is substantially Atrasentan Hydrochloride Crystalline Form 1 (e.g., >75%, >80%, >85%, >90%, >95%, >98%, >99%, or >99.5% Form 1). Atrasentan Hydrochloride Crystalline Form 1 and methods of making the same are described in International Patent Application Publication No. WO 2006/034094, which is incorporated by reference herein in its entirety.

In some embodiments, Atrasentan Hydrochloride Crystalline Form 1 is characterized, when measured at about 25° C. with Cu-Kα radiation, by an X-ray powder diffraction pattern with at least three peaks (e.g., 3, 4, 5, 6, or 7) having respective 2θ values of about 8.3°, 9.7°, 10.0°, 13.0°, 15.6°, 17.2° or 19.5°. In certain embodiments, Atrasentan Hydrochloride Crystalline Form 1 is characterized, when measured at about 25° C. with Cu-Kα radiation, by an X-ray powder diffraction pattern with at least three peaks having respective 2θ values of about 8.3°, 9.7°, 10.0°, 13.0°, 15.6°, 17.2° or 19.5°, and essentially without peaks having 2θ values below about 6.2° and/or between about 6.6° and 8.0°.

In some embodiments, Atrasentan Hydrochloride Crystalline Form 1 is characterized in the orthorhombic crystal system and P2₁2₁2₁ space group, when measured at about 25° C. with Cu-Kα radiation, by lattice parameters a, b and c of 17.663 Å±0.005 Å, 21.24 Å±0.01 Å and 8.005 Å±0.002 Å, respectively.

In some embodiments, Atrasentan Hydrochloride Crystalline Form 1 has substantial crystalline purity. In some embodiments, Atrasentan Hydrochloride Crystalline Form 1 has substantial chemical purity. In some embodiments, Atrasentan Hydrochloride Crystalline Form 1 has substantial diastereomeric purity.

Representative characteristic peak positions in the X-ray powder diffraction pattern of Atrasentan Hydrochloride Crystalline Form I, expressed as degrees relative to 2θ, are, when measured at about 25° C. with Cu-Kα radiation, about 8.3° ((020), 77.35%); 9.7° ((120), 76.37%); 10.0° ((200), 14.53%); 13.2° ((220), 28.03%); 13.6° ((130), 16.71%); 14.9° ((121), 38.93%); 15.8° ((310), 13.11%); 16.2° ((230), 18.09%); 17.4° ((320), 15.87%); 17.5° ((131), 37.80%); 19.6° ((240), 28.77%); 20.8° ((141), 46.26%); 23.3° ((112), 100.0%); 24.3° ((151), 52.6%); 25.3° ((341), 13.08%); and 25.9° ((132), 33.98%). Each peak position is shown with its accompanying Miller index (hkl) values and its integrated intensity (peak height). It is meant to be understood that peak heights may vary and will be dependent on variables such as the temperature, size of crystal size or morphology, sample preparation, or sample height in the analysis well of the Scintagx2 Diffraction Pattern System. It is also meant to be understood that peak positions may vary when measured with different radiation sources. For example, Cu-Kα1, Mo-Kα, Co-Kα and Fe-Kα radiation, having wavelengths of 1.54060 Å, 0.7107 Å, 1.7902 Å and 1.9373 Å, respectively, may provide peak positions that differ from those measured with Cu-Kα radiation.

In certain embodiments, the atrasentan, or a pharmaceutically acceptable salt thereof comprises Atrasentan Hydrochloride Crystalline Form 2. In certain embodiments, the atrasentan, or a pharmaceutically acceptable salt thereof is substantially Atrasentan Hydrochloride Crystalline Form 2 (e.g., >75%, >80%, >85%, >90%, >95%, >98%, >99%, or >99.5% Form 2). Atrasentan Hydrochloride Crystalline Form 2 and methods of making the same are described in International Patent Application Publication No. WO 2006/034084, which is incorporated by reference herein in its entirety.

In certain embodiments, Atrasentan Hydrochloride Crystalline Form 2 is characterized, when measured at about 25° C. with Cu-Kα radiation, by an X-ray powder diffraction pattern with peaks having respective 2θ values of about 6.7° and 22.05° and at least one peak having a respective 2θ value of about 8.4°, 15.6°, 18.0°, 18.5°, 19.8° or 20.6°.

In certain embodiments, Atrasentan Hydrochloride Crystalline Form 2 has substantial crystalline purity and is characterized, when measured at about 25° C. with Cu-Kα radiation, by an X-ray powder diffraction pattern with peaks having respective 2θ values of about 6.7° and 22.05° and at least one peak having a respective 2θ value of about 8.4°, 15.6°, 18.0°, 18.5°, 19.8° or 20.6°.

In certain embodiments, Atrasentan Hydrochloride Crystalline Form 2 has substantial crystalline purity and substantial chemical purity; and the Atrasentan Hydrochloride Crystalline Form 2 is characterized, when measured at about 25° C. with Cu-Kα radiation, by an X-ray powder diffraction pattern with peaks having respective 2θ values of about 6.7° and 22.05° and at least one peak having a respective 2θ value of about 8.4°, 15.6°, 18.0°, 18.5°, 19.8° or 20.6°.

In certain embodiments, Atrasentan Hydrochloride Crystalline Form 2 has substantial crystalline purity, substantial chemical purity, and substantial diastereomeric purity; and the Atrasentan Hydrochloride Crystalline Form 2 is characterized, when measured at about 25° C. with Cu-Kα radiation, by an X-ray powder diffraction pattern with peaks having respective 2θ values of about 6.7° and 22.05° and at least one peak having a respective 2θ value of about 8.4°, 15.6°, 18.0°, 18.5°, 19.8° or 20.6°.

In certain embodiments, the atrasentan, or a pharmaceutically acceptable salt thereof comprises Atrasentan Hydrochloride Crystalline Form 3. In certain embodiments, the atrasentan, or a pharmaceutically acceptable salt thereof is substantially Atrasentan Hydrochloride Crystalline Form 3 (e.g., >75%, >80%, >85%, >90%, >95%, >98%, >99%, or >99.5% Form 3). Atrasentan Hydrochloride Crystalline Form 3 and methods of making the same are described in International Patent Application Publication No. WO 2006/034234 and U.S. Pat. No. 9,051,301, each of which is incorporated by reference herein in its entirety.

In certain embodiments, Atrasentan Hydrochloride Crystalline Form 3 is characterized, when measured at about 25° C. with Cu-Kα radiation, by an X-ray powder diffraction pattern with peaks having respective 2θ values of about 6.7° and 21.95° and at least one peak having a respective 2θ value of about 8.4°, 15.6°, 18.0°, 18.5°, 19.8° or 20.6°.

In certain embodiments, Atrasentan Hydrochloride Crystalline Form 3 has substantial crystalline purity and is characterized, when measured at about 25° C. with Cu-Kα radiation, by an X-ray powder diffraction pattern with peaks having respective 2θ values of about 6.7° and 21.95° and at least one peak having a respective 2θ value of about 8.4°, 15.6°, 18.0°, 18.5°, 19.8° or 20.6°.

In certain embodiments, Atrasentan Hydrochloride Crystalline Form 3 has substantial crystalline purity and substantial chemical purity; and the Atrasentan Hydrochloride Crystalline Form 3 is characterized, when measured at about 25° C. with Cu-Kα radiation, by an X-ray powder diffraction pattern with peaks having respective 2θ values of about 6.7° and 21.95° and at least one peak having a respective 2θ value of about 8.4°, 15.6°, 18.0°, 18.5°, 19.8° or 20.6°.

In certain embodiments, Atrasentan Hydrochloride Crystalline Form 3 has substantial crystalline purity, substantial chemical purity, and substantial diastereomeric purity; and the Atrasentan Hydrochloride Crystalline Form 3 is characterized, when measured at about 25° C. with Cu-Kα radiation, by an X-ray powder diffraction pattern with peaks having respective 2θ values of about 6.7° and 21.95° and at least one peak having a respective 2θ value of about 8.4°, 15.6°, 18.0°, 18.5°, 19.8° or 20.6°.

In certain embodiments, the atrasentan, or a pharmaceutically acceptable salt thereof comprises amorphous atrasentan hydrochloride. In certain embodiments, atrasentan hydrochloride is substantially amorphous (e.g., >75%, >80%, >85%, >90%, >95%, >98%, >99%, or >99.5% amorphous). Amorphous atrasentan hydrochloride and methods of making the same are described in International Patent Application Publication No. WO 2006/034085, which is incorporated by reference herein in its entirety.

In certain embodiments, the amorphous atrasentan hydrochloride has substantial chemical purity. In certain embodiments, the amorphous atrasentan hydrochloride has substantial diastereomeric purity.

In some embodiments, the atrasentan, or a pharmaceutically acceptable salt thereof comprises a crystalline atrasentan mandelate salt. In certain embodiments, the atrasentan, or a pharmaceutically acceptable salt thereof is substantially a crystalline atrasentan mandelate salt (e.g., >75%, >80%, >85%, >90%, >95%, >98%, >99%, or >99.5% crystalline atrasentan mandelate salt).

In certain embodiments, the crystalline atrasentan mandelate salt is a crystalline atrasentan (S)-mandelate salt. In certain embodiments, the atrasentan (S)-mandelate salt is an anhydrous salt. In certain embodiments, the atrasentan (S)-mandelate salt is a solvated salt. In certain embodiments, the atrasentan (S)-mandelate salt is a solvated salt selected from the group consisting of an acetonitrile solvate, an ethanol solvate, and a pyridine solvate. In certain embodiments, the atrasentan (S)-mandelate salt is a hydrated salt. (a) (S)-Mandelate Salt (1:1 Stoichiometry)

In certain embodiments, the crystalline atrasentan (S)-mandelate salt is a crystalline atrasentan (S)-mandelate salt wherein the molar ratio of atrasentan to (S)-mandelate is about 1:1. In certain embodiments, the atrasentan (S)-mandelate salt is an anhydrous salt. In certain embodiments, the atrasentan (S)-mandelate salt is a solvated salt. In certain embodiments, the atrasentan (S)-mandelate salt is a solvated salt selected from the group consisting of an acetonitrile solvate, an ethanol solvate, and a pyridine solvate. In certain embodiments, the atrasentan (S)-mandelate salt is a hydrated salt. In certain embodiments, the atrasentan, or a pharmaceutically acceptable salt thereof is substantially (e.g., >75%, >80%, >85%, >90%, >95%, >98%, >99%, or >99.5%) a crystalline atrasentan (S)-mandelate salt wherein the molar ratio of atrasentan to (S)-mandelate is about 1:1.

In certain embodiments, the crystalline (S)-mandelate salt has an X-ray powder diffraction pattern comprising peaks at 5.5±0.2, 9.7±0.2, and 19.4±0.2 degrees 2θ when measured at about 25° C. with monochromatic Kα1 radiation. In certain embodiments, the crystalline (S)-mandelate salt has an X-ray powder diffraction pattern comprising peaks at 5.5±0.2, 9.7±0.2, 12.1±0.2, and 19.4±0.2 degrees 2θ when measured at about 25° C. with monochromatic Kα1 radiation. In certain embodiments, the crystalline (S)-mandelate salt has an X-ray powder diffraction pattern comprising peaks at 5.5±0.2, 9.7±0.2, 12.1±0.2, 18.0±0.2, 18.4±0.2, and 19.4±0.2 degrees 2θ when measured at about 25° C. with monochromatic Kα1 radiation. In certain embodiments, the experimental error associated with the X-ray powder diffraction peak values recited in the various embodiments above is ±0.1 degrees 2θ. In certain embodiments, the crystalline (S)-mandelate salt is an anhydrous salt. In certain embodiments, the molar ratio of atrasentan to (S)-mandelate is about 1:1.

In certain embodiments, the crystalline (S)-mandelate salt has an orthorhombic lattice type. In certain embodiments, the crystalline (S)-mandelate salt has a P212121 space group. In certain embodiments, the crystalline (S)-mandelate salt has unit cell a, b and c values of about 9.954 Å, about 11.049 Å, and about 30.861 Å, respectively. In certain embodiments, the crystalline (S)-mandelate salt has unit cell α, β and γ values of about 90°, about 90°, and about 90°, respectively. In certain embodiments, the crystalline (S)-mandelate salt has at least three or more of the following properties: (a) an orthorhombic lattice type, (b) a P212121 space group, (c) unit cell a, b and c values of about 9.954 Å, about 11.049 Å, and about 30.861 Å, respectively, and/or (d) unit cell α, β and γ values of about 90°, about 90°, and about 90°, respectively. In certain embodiments, the crystalline (S)-mandelate salt has: (a) an orthorhombic lattice type, (b) a P2₁2₁2₁ space group, (c) unit cell a, b and c values of about 9.954 Å, about 11.049 Å, and about 30.861 Å, respectively, and (d) unit cell α, β and γ values of about 90°, about 90°, and about 90°, respectively. In certain embodiments, the crystalline (S)-mandelate salt is an anhydrous salt. In certain embodiments, the molar ratio of atrasentan to (S)-mandelate is about 1:1.

### (b) (S)-Mandelate Salt (2:1 Stoichiometry)

In certain embodiments, the crystalline (S)-mandelate salt is a crystalline atrasentan (S)-mandelate salt wherein the molar ratio of atrasentan to (S)-mandelate is about 2:1. In certain embodiments, the crystalline atrasentan (S)-mandelate salt is an anhydrous salt. In certain embodiments, the crystalline atrasentan (S)-mandelate salt is a solvated salt. In certain embodiments, the crystalline atrasentan (S)-mandelate salt is a hydrated salt. In certain embodiments, the atrasentan, or a pharmaceutically acceptable salt thereof is substantially (e.g., >75%, >80%, >85%, >90%, >95%, >98%, >99%, or >99.5%) a crystalline atrasentan (S)-mandelate salt wherein the molar ratio of atrasentan to (S)-mandelate is about 2:1.

In certain embodiments, the crystalline (S)-mandelate salt has an X-ray powder diffraction pattern comprising peaks at 4.5±0.2, 8.6±0.2, and 18.1±0.2 degrees 2θ when measured at about 25° C. with monochromatic Kα1 radiation. In certain embodiments, the crystalline (S)-mandelate salt has an X-ray powder diffraction pattern comprising peaks at 4.5±0.2, 8.6±0.2, 18.1±0.2, and 18.7±0.2 degrees 2θ when measured at about 25° C. with monochromatic Kα1 radiation. In certain embodiments, the crystalline (S)-mandelate salt has an X-ray powder diffraction pattern comprising peaks at 4.5±0.2, 8.6±0.2, 9.1±0.2, 18.1±0.2, and 18.7±0.2 degrees 2θ when measured at about 25° C. with monochromatic Kα1 radiation. In certain embodiments, the experimental error associated with the X-ray powder diffraction peak values recited in the various embodiments above is ±0.1 degrees 2θ. In certain embodiments, the crystalline (S)-mandelate salt is an anhydrous salt. In certain embodiments, the crystalline (S)-mandelate salt is a hydrated salt.

In certain embodiments, the crystalline atrasentan mandelate salt is a crystalline atrasentan (R)-mandelate salt. In certain embodiments, the crystalline atrasentan (R)-mandelate salt is an anhydrous salt. In certain embodiments, the crystalline atrasentan (R)-mandelate salt is a solvated salt. In certain embodiments, the crystalline atrasentan (R)-mandelate salt is a hydrated salt.

### (c) (R)-Mandelate Salt (1:1 Stoichiometry)

In certain embodiments, the crystalline atrasentan (R)-mandelate salt is a crystalline atrasentan (R)-mandelate salt wherein the molar ratio of atrasentan to (R)-mandelate is about 1:1. In certain embodiments, the crystalline atrasentan (R)-mandelate salt is an anhydrous salt. In certain embodiments, the crystalline atrasentan (R)-mandelate salt is a solvated salt. In certain embodiments, the crystalline atrasentan (R)-mandelate salt is a hydrated salt. In certain embodiments, the atrasentan, or a pharmaceutically acceptable salt thereof is substantially (e.g., >75%, >80%, >85%, >90%, >95%, >98%, >99%, or >99.5%) a crystalline atrasentan (R)-mandelate salt wherein the molar ratio of atrasentan to (R)-mandelate is about 1:1.

In certain embodiments, the crystalline atrasentan (R)-mandelate salt has an X-ray powder diffraction pattern comprising peaks at 5.7±0.2, 11.8±0.2, and 20.9±0.2 degrees 2θ when measured at about 25° C. with monochromatic Kα1 radiation. In certain embodiments, the crystalline atrasentan (R)-mandelate salt has an X-ray powder diffraction pattern comprising peaks at 5.7±0.2, 8.2±0.2, 11.8±0.2, and 20.9±0.2 degrees 2θ when measured at about 25° C. with monochromatic Kα1 radiation. In certain embodiments, the crystalline atrasentan (R)-mandelate salt has an X-ray powder diffraction pattern comprising peaks at 5.7±0.2, 8.2±0.2, 8.6±0.2, 11.8±0.2, and 20.9±0.2 degrees 2θ when measured at about 25° C. with monochromatic Kα1 radiation. In certain embodiments, the experimental error associated with the X-ray powder diffraction peak values recited in the various embodiments above is ±0.1 degrees 2θ. In certain embodiments, the crystalline atrasentan (R)-mandelate salt is an anhydrous salt.

In some embodiments, the atrasentan, or a pharmaceutically acceptable salt thereof comprises an amorphous atrasentan mandelate salt. In certain embodiments, the atrasentan, or a pharmaceutically acceptable salt thereof is substantially an amorphous atrasentan mandelate salt (e.g., >75%, >80%, >85%, >90%, >95%, >98%, >99%, or >99.5% amorphous atrasentan mandelate salt).

In certain embodiments, the amorphous atrasentan mandelate salt is amorphous atrasentan (S)-mandelate salt. In certain embodiments, the amorphous atrasentan (S)-mandelate salt is an anhydrous salt. In certain embodiments, the amorphous atrasentan (S)-mandelate salt is a solvated salt. In certain embodiments, the amorphous atrasentan (S)-mandelate salt is a solvated salt selected from the group consisting of an acetonitrile solvate, an ethanol solvate, and a pyridine solvate. In certain embodiments, the amorphous atrasentan (S)-mandelate salt is a hydrated salt. In certain embodiments, in the amorphous atrasentan (S)-mandelate salt, the molar ratio of atrasentan and (S)-mandelate is about 1:1. In certain embodiments, in the amorphous atrasentan (S)-mandelate salt, the molar ratio of atrasentan and (S)-mandelate is about 2:1.

In certain embodiments, the amorphous atrasentan mandelate salt is amorphous atrasentan (R)-mandelate salt. In certain embodiments, the amorphous atrasentan (R)-mandelate salt is an anhydrous salt. In certain embodiments, the amorphous atrasentan (R)-mandelate salt is a solvated salt. In certain embodiments, the amorphous atrasentan (R)-mandelate salt is a solvated salt selected from the group consisting of an acetonitrile solvate, an ethanol solvate, and a pyridine solvate. In certain embodiments, the amorphous atrasentan (R)-mandelate salt is a hydrated salt. In certain embodiments, in the amorphous atrasentan (R)-mandelate salt, the molar ratio of atrasentan and (R)-mandelate is about 1:1. In certain embodiments, in the amorphous atrasentan (R)-mandelate salt, the molar ratio of atrasentan and (R)-mandelate is about 2:1.

Crystalline and amorphous atrasentan mandelate salts are further described in U.S. Pat. Nos. 8,962,675 and 9,637,476, each of which is incorporated herein by reference in its entirety.

### E. Formulation

The term “pharmaceutical composition” as used herein is intended to encompass a product or products comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure, or a pharmaceutically acceptable salt, or solvate or solvate of the salt thereof, and a pharmaceutically acceptable carrier.

In some embodiments, a pharmaceutical composition contains atrasentan, or a pharmaceutically acceptable salt thereof. In some embodiments, a pharmaceutical composition contains a SGLT-2 inhibitor. In some embodiments, a pharmaceutical composition contains atrasentan, or a pharmaceutically acceptable salt thereof, and a SGLT-2 inhibitor. The following description describes embodiments of pharmaceutical compositions of atrasentan, or a pharmaceutically acceptable salt thereof, embodiments of pharmaceutical composition of a SGLT-2 inhibitor, and embodiments of pharmaceutical composition of atrasentan, or a pharmaceutically acceptable salt thereof, and a SGLT-2 inhibitor.

The amount administered depends on the compound formulation, route of administration, etc. and is generally empirically determined, and variations will necessarily occur depending on the target, the host, and the route of administration, etc. Generally, the quantity of active compound in a unit dose of a preparation may be varied or adjusted from about 0.1 milligram (mg) to about 10 mg or from about 0.5 mg to about 2 mg, according to the particular application. For convenience, the total daily dosage may be divided and administered in portions during the day.

Pharmaceutical compositions of the present disclosure for injection comprise pharmaceutically acceptable sterile aqueous or non-aqueous solutions, dispersions, suspensions or emulsions as well as sterile powders for reconstitution into sterile injectable solutions or dispersions just prior to use. Examples of suitable aqueous and non-aqueous carriers, diluents, solvents or vehicles include water, ethanol, polyols (such as glycerol, propylene glycol, polyethylene glycol, and the like), and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactants.

These pharmaceutical compositions may also contain adjuvants such as preservative, wetting agents, emulsifying agents, and dispersing agents. Prevention of the action of micro-organisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents that delay absorption such as aluminum monostearate and gelatin. The compounds can be incorporated into slow release or targeted delivery systems such as polymer matrices, liposomes, and microspheres. Such formulations may provide more effective distribution of the compounds.

The pharmaceutical compositions that are injectable formulations can be sterilized, for example, by filtration through a bacterial-retaining filter, or by incorporating sterilizing agents in the form of sterile solid pharmaceutical compositions that can be dissolved or dispersed in sterile water or other sterile injectable medium prior to use.

Solid dosage forms of the instant pharmaceutical compositions for oral administration include capsules, tablets, pills, powders, and granules. In such solid dosage forms, the active compound(s) is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid pharmaceutical compositions of a similar type may also be employed as fillers in soft and hard-filled gelatin capsules using such excipients as lactose or milk sugar as well as high molecular weight polyethylene glycols and the like.

The solid dosage forms of the pharmaceutical compositions of tablets, dragees, capsules, pills, and granules can be prepared with coatings and shells such as enteric coatings and other pharmaceutical coatings. They may optionally contain opacifying agents and can also be of a formulation that they release the active ingredient(s) only, or preferentially, in a certain part of the intestinal tract, optionally, in a delayed manner. Examples of embedding pharmaceutical compositions which can be used include polymeric substances and waxes.

The active compounds can also be in microencapsulated form, if appropriate, with one or more of the above-mentioned excipients.

Liquid dosage forms of the instant pharmaceutical compositions for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral pharmaceutical compositions can also include adjuvants such as wetting agents, emulsifying and suspending agents, sweetening, flavoring, and perfuming agents.

Suspensions of the instant compounds, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

The compounds and compositions described herein can, for example, be administered orally or parenterally, with a dosage ranging from about 0.01 milligrams per kilogram (mg/kg) to about 0.05 mg/kg, every 4 to 120 hours, or according to the requirements of the particular drug, dosage form, and/or route of administration. The interrelationship of dosages for animals and humans (based on milligrams per meter squared of body surface) is described by Freireich et al., Cancer Chemother. Rep. 50, 219-244 (1966). Body surface area may be approximately determined from height and weight of the patient. See, e.g., Scientific Tables, Geigy Pharmaceuticals, Ardsley, N.Y., 537 (1970). In certain embodiments, the compositions are administered by oral administration or by injection. The methods herein contemplate administration of an effective amount of compound or compound composition to achieve a desired or stated effect. Typically, the pharmaceutical compositions of the present disclosure will be administered from about 1 to about 6 times per day, or alternatively, as a continuous infusion. Such administration can be used as a chronic or acute therapy.

Lower or higher doses than those recited above may be required. Specific dosage and treatment regimens for any particular patient will depend upon a variety of factors, including the activity of the specific compound employed, the age, body weight, general health status, sex, diet, time of administration, rate of excretion, drug combination, the severity and course of the disease, condition or symptoms, the patient's disposition to the disease, and the judgment of the treating physician.

Dosage forms of atrasentan, or a pharmaceutically acceptable salt thereof include from about 0.01 mg to about 10 mg (including, from about 0.1 mg to about 5 mg, from about 0.2 mg to about 4 mg, from about 0.3 mg to about 3 mg, from about 0.4 mg to about 2 mg, from about 0.5 mg to about 1.5 mg, or from about 0.6 mg to about 1 mg) of a compound of atrasentan, or a pharmaceutically acceptable salt thereof. In some embodiments, the dosage form includes about 0.1 mg, about 0.2 mg, about 0.3 mg, about 0.4 mg, about 0.5 mg, about 0.6 mg, about 0.65, about 0.7 mg, about 0.75, about 0.8 mg, about 0.85, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, about 2 mg, or any value in between, of atrasentan, or a pharmaceutically acceptable salt thereof. In some embodiments, the dosage form includes about 0.75 mg of a compound of atrasentan, or a pharmaceutically acceptable salt thereof.

The dosage forms can further include a pharmaceutically acceptable carrier and/or an additional therapeutic agent.

Appropriate dosage levels may be determined by any suitable method. Preferably, the active substance is administered at a frequency of 1 to 4 times per day for topical administration, or less often if a drug delivery system is used. Nevertheless, actual dosage levels and time course of administration of the active ingredients in the pharmaceutical compositions of the present disclosure may be varied so as to obtain an amount of the active ingredient which is effective to achieve a desired therapeutic response for a particular patient, composition and mode of administration, without being intolerably toxic to the patient. In certain cases, dosages may deviate from the stated amounts, in particular as a function of age, gender, body weight, diet and general health status of the patient, route of administration, individual response to the active ingredient, nature of the preparation, and time or interval over which administration takes place. Thus, it may be satisfactory in some cases to manage with less than the aforementioned minimum amount, whereas in other cases the stated upper limit may be exceeded. It may in the event of administration of larger amounts be advisable to divide these into multiple individual doses spread over the day.

### Exemplary Dosage Forms of Atrasentan

In some embodiments, provided herein a stable solid pharmaceutical dosage form comprising: (a) about 0.25 mg to about 1.25 mg of atrasentan, or an equivalent amount of a pharmaceutically acceptable salt thereof; wherein the weight percent of atrasentan, or pharmaceutically acceptable salt thereof, in the dosage form is from about 0.05 weight percent to about 2.0 weight percent on an atrasentan free base equivalent weight basis; and (b) a pharmaceutically acceptable diluent.

In some embodiments, provided herein a stable solid pharmaceutical dosage form comprising: (a) about 0.25 mg to about 1.25 mg of atrasentan, or an equivalent amount of a pharmaceutically acceptable salt thereof; wherein the weight percent of atrasentan, or pharmaceutically acceptable salt thereof, in the dosage form is from about 0.05 weight percent to about 2.0 weight percent on an atrasentan free base equivalent weight basis; (b) a pharmaceutically acceptable anti-oxidant; wherein the molar ratio of the anti-oxidant to atrasentan, or pharmaceutically acceptable salt thereof, is from about 10:1 to about 1:10; and (c) a pharmaceutically acceptable diluent.

In certain of these embodiments, degradation of atrasentan in the dosage form is less than degradation of atrasentan in an otherwise identical dosage form lacking the anti-oxidant when the dosage forms are stored for a storage period of six months at about 40° C. and about 75% relative humidity.

In some embodiments, the dosage form is stored during the storage period in a semi-permeable container or a substantially impermeable container. In some embodiments, the dosage form is stored during the storage period in a sealed HDPE bottle or a blister package. In some embodiments, the dosage form is stored during the storage period in a sealed HDPE bottle. In some embodiments, the dosage form is stored during the storage period in a blister package.

### (i) Atrasentan

The dosage form can comprise a free base of atrasentan, a pharmaceutically acceptable salt of atrasentan, or a combination thereof. In some embodiments, the dosage form comprises a free base of atrasentan. In some embodiments, the dosage form comprises a pharmaceutically acceptable salt of atrasentan. In some embodiments, the dosage form comprises atrasentan hydrochloride. In some embodiments, the dosage form comprises atrasentan hydrochloride having a polymorph form selected from the group consisting of amorphous atrasentan hydrochloride, Atrasentan Hydrochloride Crystalline Form 1, Atrasentan Hydrochloride Crystalline Form 2, and Atrasentan Hydrochloride Crystalline Form 3. In some embodiments, the dosage form comprises amorphous atrasentan hydrochloride. In some embodiments, the dosage form comprises Atrasentan Hydrochloride Crystalline Form 1. In some embodiments, the dosage form comprises Atrasentan Hydrochloride Crystalline form 2. In some embodiments, the dosage form comprises atrasentan hydrochloride crystalline form 3. In some embodiments, the dosage form comprises atrasentan mandelate. In certain embodiments, the dosage form comprises a crystalline atrasentan mandelate (e.g., a crystalline atrasentan (S)-mandelate and/or a crystalline atrasentan (R)-mandelate). In certain embodiments, the dosage form comprises an amorphous atrasentan mandelate (e.g., an amorphous atrasentan (S)-mandelate and/or an amorphous atrasentan (R)-mandelate). In certain of the foregoing embodiments (when the dosage form comprises a crystalline and/or amorphous atrasentan (S)- and/or (R)-mandelate), the molar ratio of atrasentan and mandelate is 1:1. In certain other embodiments, the molar ratio of atrasentan and mandelate is 2:1.

In certain embodiments, the dosage form comprises amorphous atrasentan hydrochloride; and it is substantially free (e.g., contains <10%, <5%, <1%, <0.5%, <0.1%, <0.05%) of other forms (e.g., other salts and/or other polymorphs) of atrasentan. In certain embodiments, the dosage form comprises Atrasentan Hydrochloride Crystalline Form 1; and it is substantially free of (e.g., contains <10%, <5%, <1%, <0.5%, <0.1%, <0.05%) other forms (e.g., other salts and/or other polymorphs) of atrasentan. In certain embodiments, the dosage form comprises Atrasentan Hydrochloride Crystalline form 2; and it is substantially free of other forms (e.g., other salts and/or other polymorphs) of atrasentan. In certain embodiments, the dosage form comprises atrasentan hydrochloride crystalline form 3; and it is substantially free (e.g., contains <10%, <5%, <1%, <0.5%, <0.1%, <0.05%) of other forms (e.g., other salts and/or other polymorphs) of atrasentan. In certain embodiments, the dosage form comprises crystalline atrasentan (S)-mandelate; and it is substantially free (e.g., contains <10%, <5%, <1%, <0.5%, <0.1%, <0.05%) of other forms (e.g., other salts and/or other polymorphs) of atrasentan. In certain embodiments, the dosage form comprises crystalline atrasentan (R)-mandelate; and it is substantially free (e.g., contains <10%, <5%, <1%, <0.5%, <0.1%, <0.05%) of other forms (e.g., other salts and/or other polymorphs) of atrasentan. In certain embodiments, the dosage form comprises amorphous atrasentan (S)-mandelate; and it is substantially free (e.g., contains <10%, <5%, <1%, <0.5%, <0.1%, <0.05%) of other forms (e.g., other salts and/or other polymorphs) of atrasentan. In certain embodiments, the dosage form comprises amorphous atrasentan (R)-mandelate; and it is substantially free (e.g., contains <10%, <5%, <1%, <0.5%, <0.1%, <0.05%) of other forms (e.g., other salts and/or other polymorphs) of atrasentan.

In some embodiments, the weight percent of atrasentan, or pharmaceutically acceptable salt thereof, in the dosage form is from about 0.1 weight percent to about 2.0 weight percent on an atrasentan free base equivalent weight basis. In some embodiments, the weight percent of atrasentan, or pharmaceutically acceptable salt thereof, in the dosage form is from about 0.2 weight percent to about 1.0 weight percent on an atrasentan free base equivalent weight basis. In some embodiments, the weight percent of atrasentan, or pharmaceutically acceptable salt thereof, in the dosage form is from about 0.3 weight percent to about 0.8 weight percent on an atrasentan free base equivalent weight basis. In some embodiments, the weight percent of atrasentan, or pharmaceutically acceptable salt thereof, in the dosage form is from about 0.40 weight percent to about 0.45 weight percent on an atrasentan free base equivalent weight basis. In some embodiments, the weight percent of atrasentan, or pharmaceutically acceptable salt thereof, in the dosage form is from about 0.60 weight percent to about 0.65 weight percent on an atrasentan free base equivalent weight basis.

In some embodiments, the dosage form comprises from about 0.40 mg to about 1.00 mg of atrasentan, or an equivalent amount of a pharmaceutically acceptable salt thereof. In some embodiments, the dosage form comprises from about 0.40 mg to about 0.85 mg of atrasentan, or an equivalent amount of a pharmaceutically acceptable salt thereof. In some embodiments, the dosage form comprises about 0.50 mg of atrasentan, or an equivalent amount of a pharmaceutically acceptable salt thereof. In some embodiments, the dosage form comprises about 0.75 mg of atrasentan, or an equivalent amount of a pharmaceutically acceptable salt thereof.

### (ii) Diluent

Suitable diluents for use in the disclosed dosage forms include, but are not limited to, lactose (such as lactose monohydrate, lactose anhydrous, and PHARMATOSE® DCL21), sucrose, glucose, mannitol, sorbitol, isomalt, microcrystalline cellulose (such as AVICEL® PH101 and AVICEL® PH102), silicified microcrystalline cellulose (such as PROSOLV® SMCC 50 and SMCC 90), dicalcium phosphate, starches, and combinations thereof. In some embodiments, the diluent is selected from the group consisting of lactose, mannitol, isomalt, microcrystalline cellulose, dicalcium phosphate, and combinations thereof. In some embodiments, the diluent is lactose.

In some embodiments, the weight percent of the diluent in the dosage form is from about 70 weight percent to about 99 weight percent. In some embodiments, the weight percent of the diluent in the dosage form is from about 80 weight percent to about 99 weight percent. In some embodiments, the weight percent of the diluent in the dosage form is from about 85 weight percent to about 99 weight percent. In certain of the foregoing embodiments, the diluent is selected from the group consisting of lactose, mannitol, isomalt, and combinations thereof. As a non-limiting example, the diluent can be lactose.

### (iii) Binder

In some embodiments, the dosage form further comprises a pharmaceutically acceptable binder (e.g., polymeric binder). Suitable binders for use in the disclosed dosage forms include, but are not limited to, celluloses, such as hydroxypropyl methylcellulose (e.g., Hypromellose E5 (Premium LV)), hydroxypropyl ethylcellulose, and hydroxypropyl cellulose, and other pharmaceutically acceptable substances with cohesive properties. In some embodiments, the binder is selected from the group consisting of hydroxymethylpropylcellulose, hydroxyethylpropylcellulose, and hydroxypropylcellulose. In some embodiments, the binder is hydroxypropyl methylcellulose. In some embodiments, the binder is hydroxypropylcellulose. In some embodiments, the binder is hydroxyethylpropylcellulose.

In some embodiments, the dosage form further comprises a pharmaceutically acceptable binder; and the weight percent of the binder in the dosage form is from about 1.0 weight percent to about 10.0 weight percent. In some embodiments, the weight percent of the binder in the dosage form is from about 1.0 weight percent to about 8.0 weight percent. In some embodiments, the weight percent of the binder in the dosage form is from about 1.0 weight percent to about 5.0 weight percent. In certain of the foregoing embodiments, the binder is a polymeric binder selected from the group consisting of hydroxymethylpropylcellulose, hydroxyethylpropylcellulose, and hydroxypropylcellulose

In some embodiments, the dosage form further comprises a pharmaceutically acceptable binder; and the weight to weight ratio of the binder to atrasentan, or pharmaceutically acceptable salt thereof, is from about 2:1 to about 25:1 on an atrasentan free base equivalent weight basis. In some embodiments, the weight to weight ratio of the binder to the atrasentan, or pharmaceutically acceptable salt thereof, is from about 1:1 to about 20:1 on an atrasentan free base equivalent weight basis. In some embodiments, the weight to weight ratio of the binder to the atrasentan, or pharmaceutically acceptable salt thereof, is from about 1:1 to about 15:1 on an atrasentan free base equivalent weight basis. In certain embodiments, the binder is a polymeric binder selected from the group consisting of hydroxymethylpropylcellulose, hydroxyethylpropylcellulose, and hydroxypropylcellulose;

### (iv) Disintegrant

In some embodiments, the dosage form optionally comprises a pharmaceutically acceptable disintegrant. Suitable disintegrants for use in the disclosed dosage forms include, but are not limited to, cross-linked polyvinyl pyrrolidone (such as POLYPLASDONE™ XL), corn starch, potato starch, maize starch and modified starches (including sodium starch glycolate), agar-agar, alginic acids, microcrystalline cellulose, sodium croscarmellose, and combinations thereof. In some embodiments, the disintegrant is selected from the group consisting of crospovidone, sodium starch glycolate, and sodium croscarmellose. In some embodiments, the disintegrant is a cross-linked polyvinyl pyrrolidone. In some embodiments, the disintegrant is crospovidone.

In some embodiments, the dosage form further comprises a pharmaceutically acceptable disintegrant. In certain embodiments, the weight percent of the disintegrant in the dosage form is from about 1.0 weight percent to about 10.0 weight percent. In some embodiments, the weight percent of the disintegrant in the dosage form is from about 1.0 weight percent to about 6.0 weight percent. In some embodiments, the weight percent of the disintegrant in the dosage form is from about 1.0 weight percent to about 4.0 weight percent. In certain of the foregoing embodiments, the disintegrant is crospovidone.

In some embodiments, the dosage form further comprises a pharmaceutically acceptable disintegrant and the weight to weight ratio of the disintegrant to the anti-oxidant (e.g., L-cysteine), or pharmaceutically acceptable salt or ester thereof, is from about 60:1 to about 3:1. In some embodiments, the weight to weight ratio of the disintegrant to the anti-oxidant (e.g., L-cysteine), or pharmaceutically acceptable salt or ester thereof, is from about 50:1 to about 4:1. In some embodiments, the weight to weight ratio of the disintegrant to the anti-oxidant (e.g., L-cysteine), or pharmaceutically acceptable salt or ester thereof, is from about 35:1 to about 5:1.

### (v) Additional Excipients

In further embodiments, the dosage form optionally comprises a pharmaceutically acceptable lubricant and/or glidant. Suitable lubricants and glidants for use in the disclosed dosage forms include, but are not limited to, silicon dioxide (such as SYLOID® 244FP and AEROSIL® 200), glyceryl behenate (such as COMPRITOL®), talc, stearic acid, solid polyethylene glycols, silica gel and mixtures thereof, and other substances with lubricating or gliding properties. In certain embodiments, the lubricant is glyceryl behenate (such as COMPRITOL®). In certain embodiments, the glidant is silicon dioxide (such as SYLOID® 244FP). In certain embodiments, the lubricant is glyceryl behenate and the glidant is silicon dioxide.

In some embodiments, the dosage form further comprises a pharmaceutically acceptable glidant. In another aspect, the weight percent of the glidant in the dosage form is from about 0.1 weight percent to about 1.5 weight percent. In some embodiments, the weight percent of the glidant in the dosage form is from about 0.1 weight percent to about 1.0 weight percent. In some embodiments, the weight percent of the glidant in the dosage form is from about 0.1 weight percent to about 0.8 weight percent. In some embodiments, the glidant is silicon dioxide.

In some embodiments, the dosage form further comprises a pharmaceutically acceptable lubricant. In some embodiments, the dosage form further comprises a pharmaceutically acceptable, hydrophobic lubricant. In some embodiments, the weight percent of the lubricant in the dosage form is from about 0.05 weight percent to about 5.0 weight percent. In some embodiments, the weight percent of the lubricant in the dosage form is from about 0.2 weight percent to about 3.0 weight percent. In some embodiments, the weight percent of the lubricant in the dosage form is from about 0.5 weight percent to about 2.0 weight percent. In certain embodiments, the lubricant is glyceryl behenate.

In some embodiments, the dosage form further comprises a disintegrant, a glidant, and a lubricant.

### (vi) Anti-Oxidant

Suitable anti-oxidants for use in the disclosed dosage forms include anti-oxidants that function as reducing agents and are oxidized to pharmaceutically acceptable reduced products in the dosage form. In some embodiments, the anti-oxidant has an oxidation reduction potential less than the oxidation reduction potential of atrasentan (i.e., an oxidation reduction potential less than about 900 mV) and greater than about 550 mV. In some embodiments, the anti-oxidant has an oxidation reduction potential less than about 550 mV. In some embodiments, the anti-oxidant has an oxidation reduction potential from about 1 mV to about 550 mV. In some embodiments, the solubility of the anti-oxidant in water at about 25° C. is greater than about 24 mg/mL. In some embodiments, the anti-oxidant is an amino acid, or a pharmaceutically acceptable salt or ester thereof. In some embodiments, the anti-oxidant is cysteine. In some embodiments, the anti-oxidant is L-cysteine, or a pharmaceutically acceptable salt or ester thereof. In some embodiments, the anti-oxidant is selected from the group consisting of L-cysteine hydrochloride monohydrate, L-cysteine hydrochloride anhydrate, and L-cysteine ethyl ester. In some embodiments, the dosage form comprises L-cysteine hydrochloride monohydrate.

In some embodiments, the weight percent of the anti-oxidant in the dosage form is from about 0.05 weight percent to about 1.0 weight percent. In some embodiments, the weight percent of the anti-oxidant in the dosage form is from about 0.07 weight percent to about 0.7 weight percent. In some embodiments, the weight percent of the anti-oxidant in the dosage form is from about 0.09 weight percent to about 0.5 weight percent.

In some embodiments, the molar ratio of the anti-oxidant to atrasentan, or pharmaceutically acceptable salt thereof, is from about 10:1 to about 1:10. In some embodiments, the molar ratio of the anti-oxidant to atrasentan, or pharmaceutically acceptable salt thereof, in the dosage form is from about 5:1 to about 1:5. In some embodiments, the molar ratio of the anti-oxidant to atrasentan, or pharmaceutically acceptable salt thereof, is from about 2:1 to about 1:2. In some embodiments, the molar ratio of the anti-oxidant to atrasentan, or pharmaceutically acceptable salt thereof, is about 1:1.

In some embodiments, the anti-oxidant is L-cysteine, or a pharmaceutically acceptable salt thereof. In certain embodiments, the weight percent of the L-cysteine, or pharmaceutically acceptable salt or ester thereof, in the dosage form is from about 0.05 weight percent to about 1.0 weight percent. In certain embodiments, the weight percent of the L-cysteine, or pharmaceutically acceptable salt or ester thereof, in the dosage form is from about 0.07 weight percent to about 0.7 weight percent. In certain embodiments, the weight percent of the L-cysteine, or pharmaceutically acceptable salt or ester thereof, in the dosage form is from about 0.09 weight percent to about 0.5 weight percent.

In certain embodiments, the molar ratio of the L-cysteine, or pharmaceutically acceptable salt or ester thereof, to atrasentan, or pharmaceutically acceptable salt thereof, in the dosage form is from about 10:1 to about 1:10. In certain embodiments, the molar ratio of the L-cysteine, or pharmaceutically acceptable salt or ester thereof, to atrasentan, or pharmaceutically acceptable salt thereof, in the dosage form is from about 5:1 to about 1:5. In certain embodiments, the molar ratio of the L-cysteine, or pharmaceutically acceptable salt or ester thereof, to atrasentan, or pharmaceutically acceptable salt thereof, is from about 2:1 to about 1:2. In certain embodiments, the molar ratio of the L-cysteine, or pharmaceutically acceptable salt or ester thereof, to atrasentan, or pharmaceutically acceptable salt thereof, about 1:1.

In certain embodiments, the anti-oxidant is selected from the group consisting of L-cysteine hydrochloride monohydrate, L-cysteine hydrochloride anhydrate, and L-cysteine ethyl ester. In some embodiments, the dosage form comprises L-cysteine hydrochloride monohydrate.

### (vii) Additional Embodiments

In some embodiments, the dosage form comprises atrasentan, or a pharmaceutically acceptable salt thereof, and an anti-oxidant. In certain of these embodiments, the anti-oxidant is L-cysteine, or pharmaceutically acceptable salt or ester thereof. In some embodiments, the molar ratio of the anti-oxidant (e.g., L-cysteine, or pharmaceutically acceptable salt or ester thereof) is from about 5:1 to about 1:5. In certain of the foregoing embodiments, the dosage form further comprises a pharmaceutically acceptable polymeric binder selected from the group consisting of hydroxymethylpropylcellulose, hydroxyethylpropylcellulose, and hydroxypropylcellulose; the molar ratio of the anti-oxidant (e.g., L-cysteine, or pharmaceutically acceptable salt or ester thereof) to atrasentan, or pharmaceutically acceptable salt thereof, is from about 5:1 to about 1:5; and the weight to weight ratio of the binder to atrasentan, or pharmaceutically acceptable salt thereof, is from about 1:1 to about 20:1 on an atrasentan free base equivalent weight basis. In some embodiments, this dosage form further comprises a disintegrant and the weight to weight ratio of the disintegrant to the anti-oxidant (e.g., L-cysteine, or pharmaceutically acceptable salt or ester thereof) is from about 60:1 to about 3:1. In some embodiments, the weight percent of atrasentan, or pharmaceutically acceptable salt thereof, in this dosage form is from about 0.2 weight percent to about 1.0 weight percent on an atrasentan free base equivalent weight basis. In some embodiments, this dosage form comprises from about 0.40 mg to about 0.85 mg of atrasentan, or an equivalent amount of a pharmaceutically acceptable salt thereof.

In some embodiments, the dosage form comprises a pharmaceutically acceptable polymeric binder selected from the group consisting of hydroxymethylpropylcellulose, hydroxyethylpropylcellulose, and hydroxypropylcellulose; the molar ratio of the anti-oxidant (e.g., L-cysteine, or a pharmaceutically acceptable salt or ester thereof), to atrasentan, or pharmaceutically acceptable salt thereof, is from about 2:1 to about 1:2; and the weight to weight ratio of the binder to atrasentan, or pharmaceutically acceptable salt thereof, is from about 1:1 to about 15:1 on an atrasentan free base equivalent weight basis. In some embodiments, the dosage form further comprises a disintegrant and the weight to weight ratio of the disintegrant to the anti-oxidant (e.g., L-cysteine, or a pharmaceutically acceptable salt or ester thereof), is from about 50:1 to about 4:1. In some embodiments, the weight percent of atrasentan, or pharmaceutically acceptable salt thereof, in this dosage form is from about 0.2 weight percent to about 1.0 weight percent on an atrasentan free base equivalent weight basis. In some embodiments, this dosage form comprises from about 0.40 mg to about 0.85 mg of atrasentan, or an equivalent amount of a pharmaceutically acceptable salt thereof.

In some embodiments, the dosage form comprises a pharmaceutically acceptable polymeric binder selected from the group consisting of hydroxymethylpropylcellulose, hydroxyethylpropylcellulose, and hydroxypropylcellulose; the molar ratio of the anti-oxidant (e.g., L-cysteine, or pharmaceutically acceptable salt or ester thereof), to atrasentan, or pharmaceutically acceptable salt thereof, is about 1:1; and the weight to weight ratio of the binder to atrasentan, or pharmaceutically acceptable salt thereof, is from about 1:1 to about 15:1 on an atrasentan free base equivalent weight basis. In some embodiments, this dosage form further comprises a disintegrant and the weight to weight ratio of the disintegrant to the anti-oxidant (e.g., L-cysteine, or pharmaceutically acceptable salt or ester thereof), is from about 35:1 to about 5:1. In some embodiments, the weight percent of atrasentan, or pharmaceutically acceptable salt thereof, in this dosage form is from about 0.3 weight percent to about 0.8 weight percent on an atrasentan free base equivalent weight basis. In some embodiments, this dosage form comprises from about 0.40 mg to about 0.85 mg of atrasentan, or an equivalent amount of a pharmaceutically acceptable salt thereof.

In some embodiments, the dosage form comprises a pharmaceutically acceptable polymeric binder selected from the group consisting of hydroxymethylpropylcellulose, hydroxyethylpropylcellulose, and hydroxypropylcellulose; the dosage form comprises from about 0.05 weight percent to about 1.0 weight percent of the anti-oxidant (e.g., L-cysteine, or pharmaceutically acceptable salt or ester thereof); and the dosage form comprises from about 1.0 weight percent to about 10.0 weight percent of the binder. In some embodiments, this dosage form further comprises a disintegrant and the weight percent of the disintegrant in the dosage form is from about 1.0 weight percent to about 10.0 weight percent. In some embodiments, the weight percent of atrasentan, or pharmaceutically acceptable salt thereof, in this dosage form is from about 0.1 weight percent to about 2.0 weight percent on an atrasentan free base equivalent weight basis. In some embodiments, this dosage form comprises from about 0.40 mg to about 0.85 mg of atrasentan, or an equivalent amount of a pharmaceutically acceptable salt thereof.

In some embodiments, the dosage form comprises a pharmaceutically acceptable polymeric binder selected from the group consisting of hydroxymethylpropylcellulose, hydroxyethylpropylcellulose, and hydroxypropylcellulose; the dosage form comprises from about 0.07 weight percent to about 0.70 weight percent of the anti-oxidant (e.g., L-cysteine, or pharmaceutically acceptable salt or ester thereof); and the dosage form comprises from about 1.0 weight percent to about 8.0 weight percent of the binder. In some embodiments, this dosage form further comprises a disintegrant and the weight percent of the disintegrant in the dosage form is from about 1.0 weight percent to about 6.0 weight percent. In some embodiments, the weight percent of atrasentan, or pharmaceutically acceptable salt thereof, in this dosage form is from about 0.2 weight percent to about 1.0 weight percent on an atrasentan free base equivalent weight basis. In some embodiments, this dosage form comprises from about 0.40 mg to about 0.85 mg of atrasentan, or an equivalent amount of a pharmaceutically acceptable salt thereof.

In some embodiments, the dosage form comprises a pharmaceutically acceptable polymeric binder selected from the group consisting of hydroxymethylpropylcellulose, hydroxyethylpropylcellulose, and hydroxypropylcellulose; the dosage form comprises from about 0.09 weight percent to about 0.80 weight percent of the anti-oxidant (e.g., L-cysteine, or pharmaceutically acceptable salt or ester thereof) and the dosage form comprises from about 1.0 weight percent to about 5.0 weight percent of the binder. In some embodiments, this dosage form further comprises a disintegrant and the weight percent of the disintegrant in the dosage form is from about 1.0 weight percent to about 4.0 weight percent. In some embodiments, the weight percent of atrasentan, or pharmaceutically acceptable salt thereof, in this dosage form is from about 0.3 weight percent to about 0.8 weight percent on an atrasentan free base equivalent weight basis. In some embodiments, this dosage form comprises from about 0.40 mg to about 0.85 mg of atrasentan, or an equivalent amount of a pharmaceutically acceptable salt thereof.

In some embodiments, the dosage form comprises:
(a) about 0.1 weight percent to about 2.0 weight percent of atrasentan, or pharmaceutically acceptable salt thereof, on an atrasentan free base equivalent weight basis;
(b) about 0.05 weight percent to about 1.0 weight percent of the anti-oxidant (e.g., L-cysteine, or pharmaceutically acceptable salt or ester thereof);
(c) about 75 weight percent to about 99 weight percent of the diluent;
(d) about 1.0 weight percent to about 10.0 weight percent of a pharmaceutically acceptable binder;
(e) optionally, about 1.0 weight percent to about 10.0 weight percent of a pharmaceutically acceptable disintegrant;
(f) optionally, about 0 weight percent to about 1.5 weight percent of a pharmaceutically acceptable glidant; and
(g) optionally, about 0 weight percent to about 5.0 weight percent of a pharmaceutically acceptable lubricant;
wherein the cumulative weight percent for all components of the dosage form equals 100 percent.

In some embodiments, the dosage form comprises:
(a) about 0.1 weight percent to about 2.0 weight percent of atrasentan, or pharmaceutically acceptable salt thereof, on an atrasentan free base equivalent weight basis;
(b) about 0.05 weight percent to about 1.0 weight percent of the anti-oxidant (L-cysteine, or pharmaceutically acceptable salt or ester thereof);
(c) about 75 weight percent to about 99 weight percent of the diluent;
(d) about 1.0 weight percent to about 10.0 weight percent of a pharmaceutically acceptable binder;
(e) about 1.0 weight percent to about 10.0 weight percent of a pharmaceutically acceptable disintegrant;
(f) optionally, about 0 weight percent to about 1.5 weight percent of a pharmaceutically acceptable glidant; and
(g) optionally, about 0 weight percent to about 5.0 weight percent of a pharmaceutically acceptable lubricant;
wherein the cumulative weight percent for all components of the dosage form equals 100 percent.

In some embodiments, the dosage form comprises:
(a) about 0.2 weight percent to about 1.0 weight percent of atrasentan, or pharmaceutically acceptable salt thereof, on an atrasentan free base equivalent weight basis;
(b) about 0.07 weight percent to about 0.7 weight percent of the anti-oxidant (L-cysteine, or
(c) about 82 weight percent to about 99 weight percent of the diluent;
(d) about 1.0 weight percent to about 8.0 weight percent of a pharmaceutically acceptable binder;
(e) optionally, about 1.0 weight percent to about 6.0 weight percent of a pharmaceutically acceptable disintegrant;
(f) optionally, about 0 weight percent to about 1.0 weight percent of a pharmaceutically acceptable glidant; and
(g) optionally, about 0 weight percent to about 3.0 weight percent of a pharmaceutically acceptable lubricant;
wherein the cumulative weight percent for all components of the dosage form equals 100 percent.

In some embodiments, the dosage form comprises:
(a) about 0.2 weight percent to about 1.0 weight percent of atrasentan, or pharmaceutically acceptable salt thereof, on an atrasentan free base equivalent weight basis;
(b) about 0.07 weight percent to about 0.70 weight percent of the (L-cysteine, or pharmaceutically acceptable salt or ester thereof);
(c) about 82 weight percent to about 99 weight percent of the diluent;
(d) about 1.0 weight percent to about 8.0 weight percent of a pharmaceutically acceptable binder;
(e) about 1.0 weight percent to about 6.0 weight percent of a pharmaceutically acceptable disintegrant;
(f) optionally, about 0 weight percent to about 1.0 weight percent of a pharmaceutically acceptable glidant; and
(g) optionally, about 0 weight percent to about 3.0 weight percent of a pharmaceutically acceptable lubricant;
wherein the cumulative weight percent for all components of the dosage form equals 100 percent.

In some embodiments, the dosage form comprises:
(a) about 0.3 weight percent to about 0.8 weight percent of atrasentan, or pharmaceutically acceptable salt thereof, on an atrasentan free base equivalent weight basis;
(b) about 0.09 weight percent to about 0.50 weight percent of the (L-cysteine, or
(c) about 87 weight percent to about 99 weight percent of a pharmaceutically acceptable diluent;
(d) about 1.0 weight percent to about 5.0 weight percent of a pharmaceutically acceptable binder;
(e) optionally, about 1.0 weight percent to about 4.0 weight percent of a pharmaceutically acceptable disintegrant;
(f) optionally, about 0 weight percent to about 0.75 weight percent of a pharmaceutically acceptable glidant; and
(g) optionally, about 0 weight percent to about 2.0 weight percent of a pharmaceutically acceptable lubricant;
wherein the cumulative weight percent for all components of the dosage form equals 100 percent.

In some embodiments, the dosage form comprises:
(a) about 0.3 weight percent to about 0.8 weight percent of atrasentan, or pharmaceutically acceptable salt thereof, on an atrasentan free base equivalent weight basis;
(b) about 0.09 weight percent to about 0.50 weight percent of the (L-cysteine, or pharmaceutically acceptable salt or ester thereof);
(c) about 87 weight percent to about 99 weight percent of a pharmaceutically acceptable diluent;
(d) about 1.0 weight percent to about 5.0 weight percent of a pharmaceutically acceptable binder;
(e) about 1.0 weight percent to about 4.0 weight percent of a pharmaceutically acceptable disintegrant;
(f) optionally, about 0 weight percent to about 0.75 weight percent of a pharmaceutically acceptable glidant; and
(g) optionally, about 0 weight percent to about 2.0 weight percent of a pharmaceutically acceptable lubricant;
wherein the cumulative weight percent for all components of the dosage form equals 100 percent.

In some embodiments, the dosage form comprises:
(a) about 0.1 weight percent to about 2.0 weight percent of atrasentan, or pharmaceutically acceptable salt thereof, on an atrasentan free base equivalent weight basis;
(b) about 75 weight percent to about 99 weight percent of the diluent;
(c) about 1.0 weight percent to about 10.0 weight percent of a pharmaceutically acceptable binder;
(d) optionally, about 1.0 weight percent to about 10.0 weight percent of a pharmaceutically acceptable disintegrant;
(e) optionally, about 0 weight percent to about 1.5 weight percent of a pharmaceutically acceptable glidant; and
(f) optionally, about 0 weight percent to about 5.0 weight percent of a pharmaceutically acceptable lubricant;
wherein the cumulative weight percent for all components of the dosage form equals 100 percent.

In some embodiments, the dosage form comprises:
(a) about 0.1 weight percent to about 2.0 weight percent of atrasentan, or pharmaceutically acceptable salt thereof, on an atrasentan free base equivalent weight basis;
(b) about 75 weight percent to about 99 weight percent of the diluent;
(c) about 1.0 weight percent to about 10.0 weight percent of a pharmaceutically acceptable binder;
(d) about 1.0 weight percent to about 10.0 weight percent of a pharmaceutically acceptable disintegrant;
(e) optionally, about 0 weight percent to about 1.5 weight percent of a pharmaceutically acceptable glidant; and
(f) optionally, about 0 weight percent to about 5.0 weight percent of a pharmaceutically acceptable lubricant;
wherein the cumulative weight percent for all components of the dosage form equals 100 percent.

In some embodiments, the dosage form comprises:
(a) about 0.2 weight percent to about 1.0 weight percent of atrasentan, or pharmaceutically acceptable salt thereof, on an atrasentan free base equivalent weight basis;
(b) about 82 weight percent to about 99 weight percent of the diluent;
(c) about 1.0 weight percent to about 8.0 weight percent of a pharmaceutically acceptable binder;
(d) optionally, about 1.0 weight percent to about 6.0 weight percent of a pharmaceutically acceptable disintegrant;
(e) optionally, about 0 weight percent to about 1.0 weight percent of a pharmaceutically acceptable glidant; and
(f) optionally, about 0 weight percent to about 3.0 weight percent of a pharmaceutically acceptable lubricant;
wherein the cumulative weight percent for all components of the dosage form equals 100 percent.

In some embodiments, the dosage form comprises:
(a) about 0.2 weight percent to about 1.0 weight percent of atrasentan, or pharmaceutically acceptable salt thereof, on an atrasentan free base equivalent weight basis;
(b) about 82 weight percent to about 99 weight percent of the diluent;
(c) about 1.0 weight percent to about 8.0 weight percent of a pharmaceutically acceptable binder;
(d) about 1.0 weight percent to about 6.0 weight percent of a pharmaceutically acceptable disintegrant;
(e) optionally, about 0 weight percent to about 1.0 weight percent of a pharmaceutically acceptable glidant; and
(f) optionally, about 0 weight percent to about 3.0 weight percent of a pharmaceutically acceptable lubricant;
wherein the cumulative weight percent for all components of the dosage form equals 100 percent.

In some embodiments, the dosage form comprises:
(a) about 0.3 weight percent to about 0.8 weight percent of atrasentan, or pharmaceutically acceptable salt thereof, on an atrasentan free base equivalent weight basis;
(b) about 87 weight percent to about 99 weight percent of a pharmaceutically acceptable diluent;
(c) about 1.0 weight percent to about 5.0 weight percent of a pharmaceutically acceptable binder;
(d) optionally, about 1.0 weight percent to about 4.0 weight percent of a pharmaceutically acceptable disintegrant;
(e) optionally, about 0 weight percent to about 0.75 weight percent of a pharmaceutically acceptable glidant; and
(f) optionally, about 0 weight percent to about 2.0 weight percent of a pharmaceutically acceptable lubricant;
wherein the cumulative weight percent for all components of the dosage form equals 100 percent.

In some embodiments, the dosage form comprises:
(a) about 0.3 weight percent to about 0.8 weight percent of atrasentan, or pharmaceutically acceptable salt thereof, on an atrasentan free base equivalent weight basis;
(b) about 87 weight percent to about 99 weight percent of a pharmaceutically acceptable diluent;
(c) about 1.0 weight percent to about 5.0 weight percent of a pharmaceutically acceptable binder;
(d) about 1.0 weight percent to about 4.0 weight percent of a pharmaceutically acceptable disintegrant;
(e) optionally, about 0 weight percent to about 0.75 weight percent of a pharmaceutically acceptable glidant; and
(f) optionally, about 0 weight percent to about 2.0 weight percent of a pharmaceutically acceptable lubricant;
wherein the cumulative weight percent for all components of the dosage form equals 100 percent.

In some embodiments, the dosage form satisfies one or more of the following conditions:
(a) the diluent is lactose;
(b) the dosage form comprises a pharmaceutically acceptable binder and the binder is hydroxypropyl methylcellulose;
(c) the dosage form comprises a pharmaceutically acceptable disintegrant and the disintegrant is crospovidone;
(d) the dosage form comprises a pharmaceutically acceptable glidant and the glidant is silicon dioxide;
(e) the dosage form comprises a pharmaceutically acceptable lubricant and the lubricant is glyceryl behenate.

In some embodiments, the dosage form is a solid pharmaceutical dosage form comprising from about 0.25 mg to about 1.25 mg of the atrasentan, or a pharmaceutically acceptable salt thereof (e.g., atrasentan hydrochloride) on an atrasentan parent equivalent weight basis. In some embodiments, the pharmaceutical composition comprises from about 0.40 mg to about 1.00 mg of atrasentan, or a pharmaceutically acceptable salt thereof (e.g., atrasentan hydrochloride) on an atrasentan parent equivalent weight basis. In some embodiments, the pharmaceutical composition comprises from about 0.40 mg to about 0.85 mg of atrasentan, or a pharmaceutically acceptable salt thereof (e.g., atrasentan hydrochloride) on an atrasentan parent equivalent weight basis. In some embodiments, the pharmaceutical composition comprises from about 0.50 mg of atrasentan, or a pharmaceutically acceptable salt thereof (e.g., atrasentan hydrochloride) on an atrasentan parent equivalent weight basis. In some embodiments, the pharmaceutical composition comprises from about 0.75 mg of atrasentan, or a pharmaceutically acceptable salt thereof (e.g., atrasentan hydrochloride) on an atrasentan parent equivalent weight basis. In certain of the foregoing embodiments, the dosage form is a tablet.

In some embodiments, the dosage form is a tablet. In some embodiments, the tablet has a weight from about 37.5 mg to about 1500 mg. In some embodiments, the tablet has a weight from about 50 mg to about 750 mg. In some embodiments, the tablet has a weight from about 50 mg to about 250 mg. In some embodiments, the tablet has a weight from about 75 mg to about 500 mg. In some embodiments, the tablet has a weight from about 75 mg to about 150 mg. In some embodiments, the tablet has a weight from about 100 mg to about 250 mg. In some embodiments, the tablet has a weight from about 100 mg to about 230 mg. In some embodiments, the tablet has a water content is below about 10%. In certain embodiments, the tablet has a water content of about 4% to about 6% (e.g., about 4% to about 5%).

In general, the tablet optionally can be surrounded or coated with at least one non-rate-controlling layer. The non-rate-controlling layer can be formed as a single layer, coating or membrane or a plurality of single layers, coatings or membranes. The functions of the non-rate-controlling layer can include, for example, providing further stability for the atrasentan, serving as a process aid and/or as a cosmetic enhancement for the formulation, and/or acting as a masking agent to reduce any undesired odor associated with the formulation (such as the odor commonly associated with L-cysteine).

When the dosage form comprises a non-rate-controlling layer, the non-rate-controlling layer can be made of one or more polymers, as well as, other ingredients known in the art, such as, but not limited to, plasticizers, pigments/opacifiers, waxes, etc. Examples of polymers that can be used include, but are not limited to, hydroxypropyl methylcellulose, hydroxypropyl cellulose, methylcellulose, polyvinyl alcohol and polyethylene glycol. Examples of plasticizers that can be used include, but are not limited to, polyethylene glycol(s), glycerin, triacetin, triethyl citrate, diethyl phthalate, L-cysteine, and mineral oils. Examples of pigments/opacifiers that can be used include, but are not limited to, water soluble dyes (for example, sunset yellow, quinoline yellow, erythrosine, and tartrazine), pigments (for example, aluminum lakes, titanium oxides, iron oxides and talc), and natural products (for example, riboflavin, carotenoids, chlorophyll, anthocyanins, and carmine). An example of a wax that can be used includes, but is not limited to, a paraffin wax.

In some embodiments, the dosage form is a tablet coated with a pharmaceutically acceptable polymer.

In some embodiments, the dosage form is a capsule.

In some embodiments, the dosage form is packaged in a semi-permeable container. In some embodiments, the semi-permeable container is a blister pack.

In some embodiments, the dosage form is packaged in a substantially impermeable container.

In some embodiments, the dosage form is an immediate release dosage form. In some embodiments, the dosage form is an immediate release tablet and releases at least about 85% of the atrasentan, or pharmaceutically acceptable salt thereof, within about 45 minutes as determined in an in vitro dissolution test conducted using a USP Dissolution Apparatus 2 (Paddle Apparatus), a 0.01N hydrochloric acid dissolution medium, and a paddle rotation of 50 RPM. In some embodiments, the dosage form is an immediate release tablet and releases at least about 75% of the atrasentan, or pharmaceutically acceptable salt thereof, within about 30 minutes.

In some embodiments, the dosage form comprises less than about 1.0 weight percent of total impurities resulting from degradation of the atrasentan, or pharmaceutically acceptable salt thereof, after a storage period of six months at about 40° C. and about 75% relative humidity. In some embodiments, degradation of the atrasentan, or pharmaceutically acceptable salt thereof, is analyzed using high-performance liquid chromatography.

In some embodiments, the dosage form comprises less than about 0.6 weight percent of any single impurity resulting from degradation of the atrasentan, or pharmaceutically acceptable salt thereof, after a storage period of six months at about 40° C. and about 75% relative humidity. In some embodiments, degradation of the atrasentan, or pharmaceutically acceptable salt thereof, is analyzed using high-performance liquid chromatography.

In some embodiments, the dosage form comprises less than about 1.0 weight percent of total impurities and less than about 0.6 weight percent of any single impurity resulting from degradation of the atrasentan, or pharmaceutically acceptable salt thereof, after a storage period of six months at about 40° C. and about 75% relative humidity. In some embodiments, degradation of the atrasentan, or pharmaceutically acceptable salt thereof, is analyzed using high-performance liquid chromatography.

In certain embodiments, the dosage form is selected from the group consisting of:

Tablet Core Composition

| Ingredient | Weight/Weight % | mg/Tablet |
|---|---|---|
| Atrasentan | 0.31 | 0.37^{a} |
| Monohydrochloride | | |
| Lactose Monohydrate | 91.19 | 109.4 |
| (Regular) | | |
| Hypromellose E5 (Premium | 3.00 | 3.6 |
| IV) | | |
| Crospovidone | 3.50 | 4.2 |
| (Polyplasdone ™ XL) | | |
| Silicon Dioxide (SYLOID ®) | 0.50 | 0.6 |
| Glyceryl Behenate | 1.50 | 1.8 |
| (COMPRITOL ®) | | |
| Purified Water^{b} | n/a | n/a |
| Total | 100% | 120 mg |

Film Coated Table Composition

| Ingredient | Weight/Weight %^{c} | mg/Tablet^{d} |
|---|---|---|
| PEG1450 | 3 | 0.1 |
| Hypromellose E3 (Premium | 97 | 3.5 |
| IV) | | |
| Purified Water | n/a | n/a |

^{a}Atrasentan monohydrochloride salt factor = 1.07 (i.e., 0.35 mg free base × 1.07 = 0.37 mg salt).
^{b}Granulation suspension medium. Less than 2% in final product.
^{c}Based on aqueous solution of 10% solids.
^{d}Based on a 120 mg tablet weight with a coating weight gain of 3%.

Tablet Core Composition

| Ingredient | Weight/Weight % | mg/Tablet |
|---|---|---|
| Atrasentan | 0.4460 | 0.5350³ |
| Monohydrochloride | | |
| Lactose Monohydrate | 91.05 | 109.3 |
| (Regular) | | |
| Hypromellose E5 (Premium | 3.000 | 3.600 |
| IV) | | |
| Crospovidone | 3.500 | 4.200 |
| (Polyplasdone ™ XL) | | |
| Silicon Dioxide (SYLOID ® | 0.500 | 0.600 |
| 244FP) | | |
| Glyceryl Behenate | 1.500 | 1.80 |
| (COMPRITOL ®) | | |
| Purified Water^{b} | n/a | n/a |
| Total | 100% | 120 mg |

Film Coated Table Composition

| Ingredient | Weight/Weight %^{c} | mg/Tablet^{d} |
|---|---|---|
| PEG1450 | 3 | 0.1080 |
| Hypromellose E3 (Premium | 97 | 3.492 |
| IV) | | |
| Purified Water | n/a | n/a |
| Total | 100% | 123.6 mg |

^{a}Atrasentan monohydrochloride salt factor = 1.07 (i.e., 0.50 mg free base × 1.07 = 0.5350 mg salt).
^{b}Granulation suspension medium. Less than 2% in final product.
^{c}Based on aqueous solution of 10% solids.
^{d}Based on a 120 mg tablet weight with a coating weight gain of 3%.

Tablet Core Composition

| Ingredient | Weight/Weight % | mg/Tablet |
|---|---|---|
| Atrasentan | 0.6690 | 0.8025^{a} |
| Monohydrochloride | | |
| Lactose Monohydrate | 90.83 | 109.0 |
| (Regular) | | |
| Hypromellose E5 (Premium | 3.000 | 3.600 |
| IV) | | |
| Crospovidone | 3.500 | 4.200 |
| (Polyplasdone ™ XL) | | |
| Silicon Dioxide (SYLOID ® | 0.500 | 0.600 |
| 244FP) | | |
| Glyceryl Behenate | 1.500 | 1.80 |
| (COMPRITOL ®) | | |
| Purified Water^{b} | n/a | n/a |
| Total | 100% | 120 mg |

Film Coated Table Composition

| Ingredient | Weight/Weight %^{c} | mg/Tablet^{d} |
|---|---|---|
| PEG1450 | 3 | 0.1080 |
| Hypromellose E3 (Premium | 97 | 3.492 |
| IV) | | |
| Purified Water | n/a | n/a |
| Total | 100% | 123.6 mg |

^{a}Atrasentan monohydrochloride salt factor = 1.07 (i.e., 0.75 mg free base × 1.07 = 0.8025 mg salt).
^{b}Granulation suspension medium. Less than 2% in final product.
^{c}Based on aqueous solution of 10% solids.
^{d}Based on a 120 mg tablet weight with a coating weight gain of 3%.

In certain embodiments, the dosage form is selected from the group consisting of:

Tablet Core Composition

| Ingredient | Weight/Weight % | mg/Tablet |
|---|---|---|
| Atrasentan | 0.4460 | 0.5350³ |
| Monohydrochloride | | |
| Lactose Monohydrate | 90.91 | 109.1 |
| (Regular) | | |
| L-Cysteine Hydrochloride | 0.1440 | 0.1728 |
| Monohydrate | | |
| Hypromellose E5 (Premium | 3.000 | 3.600 |
| IV) | | |
| Crospovidone | 3.500 | 4.200 |
| (Polyplasdone ™ XL) | | |
| Silicon Dioxide (SYLOID ® | 0.500 | 0.600 |
| 244FP) | | |
| Glyceryl Behenate | 1.500 | 1.800 |
| (COMPRITOL ®) | | |
| Purified Water^{b} | n/a | n/a |
| Total | 100% | 120 mg |

Film Coated Table Composition

| Ingredient | Weight/Weight %^{c} | mg/Tablet^{d} |
|---|---|---|
| PEG1450 | 3 | 0.1 |
| Hypromellose E3 (Premium | 97 | 3.5 |
| IV) | | |
| Purified Water | n/a | n/a |
| Total | 100% | 123.6 mg |

^{a}Atrasentan monohydrochloride salt factor = 1.07 (i.e., 0.5 mg free base × 1.07 = 0.5350 mg salt).
^{b}Granulation suspension medium. Less than 2% in final product.
^{c}Based on aqueous solution of 10% solids.
^{d}Based on a 120 mg tablet weight with a coating weight gain of 3%.

Tablet Core Composition

| Ingredient | Weight/Weight % | mg/Tablet |
|---|---|---|
| Atrasentan | 0.6690 | 0.8025³ |
| Monohydrochloride | | |
| Lactose Monohydrate | 90.61 | 108.7 |
| (Regular) | | |
| L-Cysteine Hydrochloride | 0.216 | 0.2592 |
| Monohydrate | | |
| Hypromellose E5 (Premium | 3.000 | 3.600 |
| IV) | | |
| Crospovidone | 3.500 | 4.200 |
| (Polyplasdone ™ XL) | | |
| Silicon Dioxide (SYLOID ® | 0.500 | 0.600 |
| 244FP) | | |
| Glyceryl Behenate | 1.500 | 1.800 |
| (COMPRITOL ®) | | |
| Purified Water^{b} | n/a | n/a |
| Total | 100% | 120 mg |

| Ingredient | Weight/Weight %^{c} | mg/Tablet^{d} |
|---|---|---|
| PEG1450 | 3 | 0.1080 |
| Hypromellose E3 (Premium | 97 | 3.492 |
| IV) | | |
| Purified Water | n/a | n/a |
| Total | 100% | 123.6 mg |

^{a}Atrasentan monohydrochloride salt factor = 1.07 (i.e., 0.75 mg free base × 1.07 = 0.8025 mg salt).
^{b}Granulation suspension medium. Less than 2% in final product.
^{c}Based on aqueous solution of 10% solids.
^{d}Based on a 120 mg tablet weight with a coating weight gain of 3%.

Tablet Core Composition

| Ingredient | Weight/Weight % | mg/Tablet |
|---|---|---|
| Atrasentan | 0.31 | 0.372^{a} |
| Monohydrochloride | | |
| Lactose Monohydrate | 91.09 | 109.3 |
| (Regular) | | |
| L-Cysteine Hydrochloride | 0.0999 | 0.120 |
| Monohydrate | | |
| Hypromellose E5 (Premium | 3.00 | 3.6 |
| IV) | | |
| Crospovidone | 3.50 | 4.2 |
| (Polyplasdone ™ XL) | | |
| Silicon Dioxide (SYLOID ® | 0.50 | 0.60 |
| 244FP) | | |
| Glyceryl Behenate | 1.50 | 1.8 |
| (COMPRITOL ®) | | |
| Purified Water^{b} | n/a | n/a |
| Total | 100% | 120 mg |

Film Coated Table Composition

| Ingredient | Weight/Weight %^{c} | mg/Tablet^{d} |
|---|---|---|
| PEG1450 | 3 | 0.1 |
| Hypromellose E3 (Premium | 97 | 3.5 |
| IV) | | |
| Purified Water | n/a | n/a |
| Total | 100% | 123.6 mg |

^{a}Atrasentan monohydrochloride salt factor = 1.07 (i.e., 0.35 mg free base × 1.07 = 0.37 mg salt).
^{b}Granulation suspension medium. Less than 2% in final product.
^{c}Based on aqueous solution of 10% solids.
^{d}Based on a 120 mg tablet weight with a coating weight gain of 3%.

Certain formulations of atrasentan, or a pharmaceutically acceptable salt thereof, and methods of making the same are further described in U.S. Pat. Nos. 9,364,458 and 10,016,393, each of which is incorporated herein by reference in its entirety.

### F. Dosage and Administration of Atrasentan

In some embodiments, a dose of atrasentan, or a pharmaceutically acceptable salt thereof is about 0.001 mg per kg of the subject's body weight (mg/kg) to about 0.1 mg/kg (e.g., about 0.001 mg/kg, about 0.002 mg/kg, about 0.003 mg/kg, about 0.004 mg/kg, about 0.005 mg/kg, about 0.006 mg/kg, about 0.007 mg/kg, about 0.008 mg/kg, about 0.009 mg/kg, about 0.01 mg/kg, about 0.015 mg/kg, about 0.02 mg/kg, about 0.025 mg/kg, about 0.03 mg/kg, about 0.035 mg/kg, about 0.04 mg/kg, about 0.045 mg/kg, about 0.05 mg/kg, about 0.055 mg/kg, about 0.06 mg/kg, about 0.065 mg/kg, about 0.07 mg/kg, about 0.075 mg/kg, about 0.08 mg/kg, about 0.085 mg/kg, about 0.09 mg/kg, about 0.095 mg/kg, or about 0.10 mg/kg, or any value in between) of atrasentan or an equivalent amount of a pharmaceutically acceptable salt thereof.

In some embodiments, a dose of atrasentan, or a pharmaceutically acceptable salt thereof is about 0.1 mg to about 10 mg (e.g. about 0.1 mg, about 0.2 mg, about 0.3 mg, about 0.4 mg, about 0.5 mg, about 0.6 mg, about 0.7 mg, about 0.75 mg, about 0.8 mg, about 0.9 mg, about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 2.5 mg, about 3.0 mg, about 3.5 mg, about 4.0 mg, about 4.5 mg, about 5.0 mg, about 5.5 mg, about 6.0 mg, about 6.5 mg, about 7.0 mg, about 7.5 mg, about 8.0 mg, about 8.5 mg, about 9.0 mg, about 9.5 mg, or about 10.0 mg, or any value in between) of atrasentan or an equivalent amount of a pharmaceutically acceptable salt thereof. In certain embodiments, a dose of atrasentan, or a pharmaceutically acceptable salt thereof is about 0.75 mg (e.g., when administered once per day) of atrasentan or an equivalent amount of a pharmaceutically acceptable salt thereof. In certain embodiments, a dose of atrasentan, or a pharmaceutically acceptable salt thereof is about 0.25 mg (e.g., when administered once per day) of atrasentan or an equivalent amount of a pharmaceutically acceptable salt thereof. In certain embodiments, a dose of atrasentan, or a pharmaceutically acceptable salt thereof is about 0.35 mg (e.g., when administered once per day) of atrasentan or an equivalent amount of a pharmaceutically acceptable salt thereof. In certain embodiments, a dose of atrasentan, or a pharmaceutically acceptable salt thereof is about 1.0 mg (e.g., when administered once per day) of atrasentan or an equivalent amount of a pharmaceutically acceptable salt thereof. In certain embodiments, a dose of atrasentan, or a pharmaceutically acceptable salt thereof is about 1.25 mg (e.g., when administered once per day) of atrasentan or an equivalent amount of a pharmaceutically acceptable salt thereof. In certain embodiments, a dose of atrasentan, or a pharmaceutically acceptable salt thereof is about 1.5 mg (e.g., when administered once per day) of atrasentan or an equivalent amount of a pharmaceutically acceptable salt thereof In certain embodiments, a dose of atrasentan, or a pharmaceutically acceptable salt thereof is about 1.75 mg (e.g., when administered once per day) of atrasentan or an equivalent amount of a pharmaceutically acceptable salt thereof. In certain of these embodiments, a dose of atrasentan, or a pharmaceutically acceptable salt thereof is 0.75 mg (e.g., 1×0.75 mg tablets; or 1.5×0.50 mg tablets) of atrasentan or an equivalent amount of a pharmaceutically acceptable salt thereof, administered once per day.

In some embodiments, a dose of atrasentan, or salt or solvate thereof, contains a therapeutically effective amount of atrasentan, or salt or solvate thereof. In other embodiments, a dose of atrasentan, or salt or solvate thereof, contains less than a therapeutically effective amount of atrasentan, or salt or solvate thereof, (e.g., when multiple doses are given in order to achieve the desired clinical or therapeutic effect).

In some embodiments, the therapeutically effective amount of atrasentan, or a pharmaceutically acceptable salt thereof, is from about 0.20 mg to about 1.5 mg of atrasentan, or an equivalent amount of a pharmaceutically acceptable salt thereof. For example, the therapeutically effective amount of atrasentan, or a pharmaceutically acceptable salt thereof can be about 0.20 mg, about 0.30 mg, about 0.40 mg, about 0.50 mg, about 0.60 mg, about 0.70 mg, about 0.80 mg, about 0.90 mg, about 1.0 mg, about 1.1 mg, about 1.2 mg, about 1.3 mg, about 1.4 mg, or about 1.5 mg of atrasentan or an equivalent amount of a pharmaceutically acceptable salt thereof. In certain embodiments, the therapeutically effective amount of atrasentan, or a pharmaceutically acceptable salt thereof, is from about 0.25 mg to about 1.25 mg of atrasentan, or an equivalent amount of a pharmaceutically acceptable salt thereof. In certain embodiments, the therapeutically effective amount of atrasentan, or a pharmaceutically acceptable salt thereof, is from about 0.40 mg to about 0.85 mg of atrasentan, or an equivalent amount of a pharmaceutically acceptable salt thereof. For example, the therapeutically effective amount of atrasentan, or a pharmaceutically acceptable salt thereof can be about 0.50 mg, about 0.55 mg, about 0.60 mg, about 0.65 mg, about 0.70 mg, about 0.75 mg, about 0.80 mg, or about 0.85 mg of atrasentan or an equivalent amount of a pharmaceutically acceptable salt thereof. As a non-limiting example, the therapeutically effective amount of atrasentan or pharmaceutically acceptable salt thereof can be about 0.75 mg of atrasentan or an equivalent amount of a pharmaceutically acceptable salt thereof.

Atrasentan, or salt or solvate thereof, can be administered by any suitable route and mode. Suitable routes of administration are well known in the art and may be selected by those of ordinary skill in the art. In some embodiments, atrasentan, or a pharmaceutically acceptable salt thereof administered parenterally. Parenteral administration refers to modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and intrasternal injection and infusion. In some embodiments, the route of administration of atrasentan is oral.

In some embodiments, atrasentan is administered to the subject daily, twice daily, three times daily or four times daily. In some embodiments, atrasentan is administered to the subject every other day, once about every week or once about every three weeks. In some embodiments, atrasentan is administered to the subject once per day. In some embodiments, atrasentan is administered to the subject twice per day. In some embodiments, atrasentan is administered to the subject at a dose of about 0.75 mg once per day. In some embodiments, atrasentan is administered to the subject at a dose of 0.75 mg once per day. In some embodiments, atrasentan is administered to the subject at a dose of about 0.25 mg once per day. In some embodiments, atrasentan is administered to the subject at a dose of 0.25 mg once per day. In some embodiments, atrasentan is administered to the subject at a dose of about 0.35 mg once per day. In some embodiments, atrasentan is administered to the subject at a dose of 0.35 mg once per day. In some embodiments, atrasentan is administered to the subject at a dose of about 0.5 mg once per day. In some embodiments, atrasentan is administered to the subject at a dose of 0.5 mg once per day. In some embodiments, atrasentan is administered to the subject at a dose of about 1.0 mg once per day. In some embodiments, atrasentan is administered to the subject at a dose of 1.0 mg once per day. In some embodiments, atrasentan is administered to the subject at a dose of about 1.75 mg once per day. In some embodiments, atrasentan is administered to the subject at a dose of 1.75 mg once per day.

### G. Dosage and Administration of an SGLT-2 Inhibitor

An atrasentan, or a pharmaceutically acceptable salt thereof is administered with a therapeutically effective amount of a SGLT-2 inhibitor. In some embodiments, the SGLT-2 inhibitor is selected from the group consisting of dapagliflozin, canagliflozin, ipragliflozin, empaglifozin, bexagliflozin, licogliflozin, janagliflozin (XZP-5695), tofogliflozin, ertugliflozin, henagliflozin (SHR-3824), enavogliflozin (DWP-16001), TA-1887 (3-(4-cyclopropylbenzyl)-4-fluoro-1-(β-D-glucopyranosyl)-1H-indole), indole-N-glycoside 18 (3-(4-ethylbenzyl)-1-(β-D-glucopyranosyl)-1H-indole), sotagliflozin, luseogliflozin, sergliflozin etabonate, remogliflozin, remogliflozin etabonate, and T-1095 (((2R,3S,4S,5R,6S)-6-(2-(3-(benzofuran-5-yl)propanoyl)-3-hydroxy-5-methylphenoxy)-3,4,5-trihydroxytetrahydro-2H-pyran-2-yl) etabonate). In some embodiments, the SGLT-2 inhibitor is selected from the group consisting of bexagliflozin, canagliflozin, dapagliflozin, empagliflozin, ertugliflozin, ipragliflozin, luseogliflozin, remogliflozin, serfliflozin, licofliglozin, sotagliflozin, and tofogliflozin. In some embodiments, the SGLT-2 inhibitor is bexagliflozin. In some embodiments, the SGLT-2 inhibitor is canagliflozin. In some embodiments, the SGLT-2 inhibitor is dapagliflozin. In some embodiments, the SGLT-2 inhibitor is empagliflozin. In some embodiments, the SGLT-2 inhibitor is ertugliflozin. In some embodiments, the SGLT-2 inhibitor is ipragliflozin. In some embodiments, the SGLT-2 inhibitor is luseogliflozin. In some embodiments, the SGLT-2 inhibitor is remogliflozin. In some embodiments, the SGLT-2 inhibitor is serfliflozin. In some embodiments, the SGLT-2 inhibitor is licofliglozin. In some embodiments, the SGLT-2 inhibitor is sotagliflozin. In some embodiments, the SGLT-2 inhibitor is tofogliflozin. In some embodiments, the SGLT-2 inhibitor is dapagliflozin propylene glycol hydrate. In some embodiments, the SGLT-2 inhibitor is canagliflozin hemihydrate. In some embodiments, the SGLT-2 inhibitor is janagliflozin (XZP-5695). In some embodiments, the SGLT-2 inhibitor is TA-1887 (3-(4-cyclopropylbenzyl)-4-fluoro-1-(β-D-glucopyranosyl)-1H-indole). In some embodiments, the SGLT-2 inhibitor is henagliflozin (SHR-3824). In some embodiments, the SGLT-2 inhibitor is enavogliflozin (DWP-16001). In some embodiments, the SGLT-2 inhibitor is indole-N-glycoside 18 (3-(4-ethylbenzyl)-1-(β-D-glucopyranosyl)-1H-indole). In some embodiments, the SGLT-2 inhibitor is sotagliflozin. In some embodiments, the SGLT-2 inhibitor is sergliflozin etabonate. In some embodiments, the SGLT-2 inhibitor is remogliflozin etabonate. In some embodiments, the SGLT-2 inhibitor is T-1095 (((2R,3S,4S,5R,6S)-6-(2-(3-(benzofuran-5-yl)propanoyl)-3-hydroxy-5-methylphenoxy)-3,4,5-trihydroxytetrahydro-2H-pyran-2-yl) etabonate).

In some embodiments, the therapeutically effective amount of the SGLT-2 inhibitor administered is from about 1 mg to about 350 mg. In some embodiments, the amount of the SGLT-2 inhibitor administered is about 1 mg to about 175 mg, about 175 mg to about 350 mg, or about 90 mg to about 260 mg. In some embodiments, the amount of the SGLT-2 inhibitor administered is from about 85 mg to about 325 mg. In some embodiments, the amount of the SGLT-2 inhibitor administered is from about 1 mg to about 50 mg, about 20 mg to about 70 mg, about 50 mg to about 100 mg, about 70 mg to about 120 mg, about 90 mg to about 140 mg, about 110 mg to about 160 mg, about 130 mg to about 180 mg, about 150 mg to about 200 mg, about 170 mg to about 220 mg, about 190 mg to about 240 mg, about 210 mg to about 260 mg, about 230 mg to about 280 mg, about 250 mg to about 300 mg, about 270 mg to about 320 mg, or about 290 mg to about 350 mg. In some embodiments, the amount of the SGLT-2 inhibitor administered is about 100 mg or about 300 mg. In some embodiments, the amount of the SGLT-2 inhibitor administered is from about 1 to about 15 mg. In some embodiments, the amount of the SGLT-2 inhibitor administered is about 1 to about 10 mg or about 5 to about 15 mg. In some embodiments, the amount of the SGLT-2 inhibitor administered is from 1 mg to about 3 mg, about 2 mg to about 4 mg, about 3 mg to about 5 mg, about 4 mg to about 6 mg, about 5 mg to about 7 mg, about 6 mg to about 8 mg, about 7 mg to about 9 mg, about 8 mg to about 10 mg, about 9 mg to about 11 mg, about 10 mg to about 12 mg, about 11 mg to about 13 mg, about 12 mg to about 14 mg, or about 13 mg to about 15 mg.

In some embodiments, the SGLT-2 inhibitor is canagliflozin. In some embodiments, 100 mg or 300 mg of canagliflozin is administered. In some embodiments, 100 mg or 300 mg of canagliflozin hemihydrate is administered. In some embodiments, the SGLT-2 inhibitor is dapagliflozin. In some embodiments, the SGLT-2 inhibitor is dapagliflozin propylene glycol hydrate. In some embodiments, 5 mg or 10 mg of dapagliflozin is administered. In some embodiments, 5 mg or 10 mg of dapagliflozin propylene glycol hydrate is administered. In some embodiments, the SGLT-2 inhibitor is empagliflozin. In some embodiments, 10 mg or 25 mg of empagliflozin is administered. In some embodiments, the SGLT-2 inhibitor is ertugliflozin. In some embodiments, 5 mg or 15 mg of ertugliflozin is administered. In some embodiments, the SGLT-2 inhibitor is ipragliflozin. In some embodiments, 25 mg or 50 mg of ipragliflozin is administered. In some embodiments, the SGLT-2 inhibitor is bexagliflozin. In some embodiments, 20 mg of bexagliflozin is administered. In some embodiments, the SGLT-2 inhibitor is sotagliflozin. In some embodiments, 200 mg or 400 mg of sotagliflozin is administered. In some embodiments, the SGLT-2 inhibitor is licogliflozin. In some embodiments, 15 mg, 50 mg, 75 mg or 150 mg of licogliflozin is administered.

In some embodiments, the SGLT-2 inhibitor is a compound that inhibits both SGLT-1 and SGLT-2. In some embodiments, the SGLT-2 inhibitor is sotagliflozin or HM41322. In some embodiments, when the subject has Type 1 diabetes, the SGLT-2 inhibitor is a compound that inhibits both SGLT-1 and SGLT-2. In some embodiments, when the subject is suffering from Type 1 diabetes, the SGLT-2 inhibitor is a compound that inhibits both SGLT-1 and SGLT-2. In some embodiments, when the subject has been previously diagnosed with Type 1 diabetes, the SGLT-2 inhibitor is a compound that inhibits both SGLT-1 and SGLT-2. In some embodiments, when the subject is suffering from Type 1 diabetes, the SGLT-2 inhibitor is sotagliflozin or HM41322.

The SGLT-2 inhibitor can be administered by any suitable route and mode. Suitable routes of administration are well known in the art and may be selected by those of ordinary skill in the art. In some embodiments, the SGLT-2 inhibitor is administered orally. In some embodiments, the SGLT-2 inhibitor is administered to the subject daily, twice daily, three times daily or four times daily. In some embodiments, the SGLT-2 inhibitor is administered to the subject every other day, once about every week or once about every three weeks.

In some embodiments, the subject was previously being administered an SGLT-2 inhibitor prior to the first administration of atrasentan, or a pharmaceutically acceptable salt thereof. In some embodiments, the subject was previously being administered an SGLT-2 inhibitor prior to the first administration of atrasentan, or a pharmaceutically acceptable salt thereof, and continues being administered the SGLT-2 inhibitor during the administration of atrasentan, or a pharmaceutically acceptable salt thereof. In some embodiments, the SGLT-2 inhibitor administered prior to the first administration of atrasentan, or a pharmaceutically acceptable salt thereof, and the SGLT-2 inhibitor administered during the administration of atrasentan, or a pharmaceutically acceptable salt thereof, are the same. In some embodiments, the SGLT-2 inhibitor administered prior to the first administration of atrasentan, or a pharmaceutically acceptable salt thereof, and the SGLT-2 inhibitor administered during the administration of atrasentan, or a pharmaceutically acceptable salt thereof, are different.

### H. Combinations

In any of the embodiments described herein, various combinations of atrasentan, or a pharmaceutically acceptable salt thereof, and a SGLT-2 inhibitor, producing an effect, are contemplated. In some embodiments, the effect, for example, any of the beneficial or desired results as described herein, is greater than the sum of the effect observed when the same amount of atrasentan, or a pharmaceutically acceptable salt thereof, when co-administered, and the same amount of the SGLT-2 inhibitor when co-administered, are each administered as a monotherapy. In some embodiments, the co-administration of atrasentan, or a pharmaceutically acceptable salt thereof, and a SGLT-2 inhibitor, produce an effect, for example, a therapeutic effect using a smaller dose of either, or both, of the compounds as a monotherapy (i.e., the dose of one or both compounds is spared, relative to the dose used for monotherapy). For example, in some embodiments, co-administration produces a therapeutic effect using a smaller dose of atrasentan, or a pharmaceutically acceptable salt thereof, and/or the SGLT-2 inhibitor compared to the amount used in monotherapy. For example, in some embodiments, the dose of atrasentan, or a pharmaceutically acceptable salt thereof, administered in combination with a SGLT-2 inhibitor may be about 50% to about 90% of the dose of atrasentan, or a pharmaceutically acceptable salt thereof, administered as a monotherapy to produce the same therapeutic effect, e.g., any of the beneficial or desired results including described herein. In some embodiments, the dose of the SGLT-2 inhibitor, administered in combination with atrasentan, or a pharmaceutically acceptable salt thereof, may be about 50% to about 90% of the dose of the SGLT-2 inhibitor, administered as a monotherapy to produce the same therapeutic effect, e.g., any of the beneficial or desired results including described herein. In some embodiments, the therapeutic effect resulting from co-administration of atrasentan, or a pharmaceutically acceptable salt thereof, and a SGLT-2 inhibitor, can include treating DKD, decreasing UACR, decreasing fluid retention (body weight gain), decreasing B-type natriuretic peptide levels, stabilizing eGFR, and/or reducing the rate of decrease of eGFR, as compared to monotherapy with a therapeutically effective dose of atrasentan, or a pharmaceutically acceptable salt thereof, or a therapeutically effective dose of a SGLT-2 inhibitor alone.

### I. Diuretics

In some embodiments, atrasentan, or a pharmaceutically acceptable salt thereof, and a SGLT-2 inhibitor may be co-administered with one or more diuretics. In some embodiments, the diuretic(s) is administered to the subject prior to administration of atrasentan, or a pharmaceutically acceptable salt thereof, and a SGLT-2 inhibitor. In some embodiments, a diuretic(s) is administered to the subject prior to administration of atrasentan, or a pharmaceutically acceptable salt thereof, and a SGLT-2 inhibitor, and during administration of atrasentan, or a pharmaceutically acceptable salt thereof, and a SGLT-2 inhibitor. In some embodiments, a diuretic(s) is administered with an angiotensin converting enzyme (ACE) inhibitor(s) and/or an angiotensin II receptor blocker(s) (ARB) to the subject prior to administration of atrasentan, or a pharmaceutically acceptable salt thereof, and a SGLT-2. In some embodiments, a diuretic is administered with an ACE inhibitor(s) and/or an ARB(s) to the subject prior to administration of atrasentan, or a pharmaceutically acceptable salt thereof, and a SGLT-2 inhibitor and during administration of atrasentan, or a pharmaceutically acceptable salt thereof, and a SGLT-2 inhibitor.

In some embodiments, the diuretic is, for example, a hydrochlorothiazide (such as MICROZIDE™ or ORETIC™), hydroflumethiazide (such as SALURON™) bemetanide (such as BUMEX™), torsemide (such as DEMADEX™), metolazone (such as ZAROXOLYN™), chlorothiazide (such as DIURIL™, ESIDRIX™ or HYDRODIURIL™) triamterene (such as DYRENIUM™), ethacrynic acid (such as EDECRIN™), chlorthalidone (such as HYGROTON™), furosemide (such as LASIX™), indapamide (such as LOZOL™) or amiloride (such as MIDAMOR™ or MODURETIC™).

In some embodiments, the diuretic is a thiazide diurectic, such as chlorothiazide, chlorthalidone, hydrochlorothiazide, trichlormethiazide, indapamide, or metolazone.

In some embodiments, the diuretic(s) is a loop diuretic, such as bumetanide, ethacrynic acid, furosemide, or torsemide.

In some embodiments, the diuretic(s) is a potassium-sparing diuretics, such as amiloride, eplerenone, spironolactone, and triamterene.

### J. Angiotensin Converting Enzyme (Ace) Inhibitors and Angiotensin II Receptor Blockers (Arbs)

In some embodiments, atrasentan, or a pharmaceutically acceptable salt thereof, and a SGLT-2 inhibitor may be co-administered with one or more ACE inhibitors and/or ARBs. In some embodiments, an ACE inhibitor(s) and/or ARB(s) is administered to the subject prior to administration of atrasentan, or a pharmaceutically acceptable salt thereof, and a SGLT-2 inhibitor. In some embodiments, an ACE inhibitor(s) and/or ARB(s) is administered to the subject prior to administration of atrasentan, or a pharmaceutically acceptable salt thereof, and a SGLT-2 inhibitor and during administration of atrasentan, or a pharmaceutically acceptable salt thereof, and a SGLT-2 inhibitor. In some embodiments, an ACE inhibitor(s) and/or ARB(s) is co-administered with a diuretic to the subject prior to administration of atrasentan, or a pharmaceutically acceptable salt thereof, and a SGLT-2 inhibitor. In some embodiments, an ACE inhibitor(s) and/or ARB(s) is co-administered with a diuretic to the subject prior to administration of atrasentan, or a pharmaceutically acceptable salt thereof, and a SGLT-2 inhibitor and during administration of atrasentan, or a pharmaceutically acceptable salt thereof, and a SGLT-2 inhibitor.

In some embodiments, the angiotensin converting enzyme (ACE) inhibitor(s) is, for example, a quinapril (such as ACCUPRIL™), fosinopril, perindopril (such as ACEON™), captopril (such as CAPOTEN™), enalapril (such as VASOTEC™), ENALAPRILAT™, ramipril (such as ALTACE™), cilazapril, delapril, fosenopril (such as MONOPRIL™), zofenopril, indolapril, benazepril (such as LOTENSIN™), lisinopril (such as PRINIVIL™ or ZESTRIL™), spirapril, trandolapril (such as MAVIK™), perindep, pentopril, moexipril (such as UNIVASC™), pivopril, temocapril, omapatrilat, imidapril, rescinnamine, benazeprilat, fosinoprilat, ramiprilat, perindoprilat, quinaprilat, trandolaprilat, moexiprilat, Quinoline Yellow WS, or cilazaprilat. In some embodiments, the ACE inhibitor(s) is selected from the group consisting of: quinapril, fosinopril perindopril, captopril, enalapril, enalaprilat, ramipril, cilazapril, delapril, fosenopril, zofenopril, indolapril, benazepril, lisinopril, spirapril, trandolapril, perindep, pentopril, moexipril, rescinnamine, and pivopril.

In some embodiments, an angiotensin II receptor blocker(s) (ARB(s)) is, for example, candesartan (such as ATACAND™), candesartan cilexetil, eprosartan (such as TEVETEN™), irbesartan (such as AVEPRO™) losartan (such as COZAAR™), olmesartan, olmesartan medoxomil (such as BENICAR™) tasosartan, telmisartan (such as MICARDIS™), valsartan (such as DIOVAN™), zolasartan, azilsartan medoxomil, F1-6828K, RNH-6270, UR-7198, Way-126227, KRH-594, TAK-536, BRA-657, or TA-606. In some embodiments, the ARB is selected from the group consisting of: candesartan, candesartan cilexetil, eprosartan, irbesartan, losartan, olmesartan, olmesartan medoxomil, telmisartan, valsartan, azilsartan medoxomil, and BRA-657.

### K. Additional Therapeutic Agents

The methods of the present disclosure also contemplate treatments comprising administering atrasentan, or a pharmaceutically acceptable salt thereof, and a SGLT-2 inhibitor as described in any of the embodiments of the disclosure, in combination with one or more additional therapeutic agents. Accordingly, atrasentan, or a pharmaceutically acceptable salt thereof, and SGLT-2 inhibitor as described anywhere herein can be administered alone or in combination with one or more additional therapeutic agents. When administered in combination with one or more additional therapeutic agents, separate dosage forms can be administered to the subject or a single dosage form comprising both atrasentan, or a pharmaceutically acceptable salt thereof, a SGLT-2 inhibitor and the additional therapeutic agent(s) can be administered to the subject. If administered as separate dosage forms, the additional therapeutic agent may be administered simultaneously with the atrasentan dosage form and/or the SGLT-2 inhibitor or sequentially (in any suitable order) with the atrasentan dosage form and the SGLT-2 inhibitor.

Representative additional therapeutic agents include, for example, antihypertensive agents, therapeutic agents for diabetes or diabetic complications, and therapeutic agents for hyperlipidemia.

In some embodiments, atrasentan, or a pharmaceutically acceptable salt thereof, and a SGLT-2 inhibitor may be co-administered with one or more calcium channel blockers such as nifedipine (such as ADALAT™, ADALAT CC™, or PROCARDIA™) verapamil (such as GALAN™, COVERA-HS™, ISOPTIN SR™, or VERELAN™), diltiazem (such as CARDIZEM™, CARDIZEM CD™, CARDIZEM LA™, CARDIZEM SR™, DILACOR™, TIAMATE™, or TIAZAC™), isradipine (such as DYNACIRC™ or DYNACIRC CR™), amlodipine (such as NORVASC™), felodipine (such as PLENDIL™) nisoldipine (such as SULAR™), bepridil (such as VASCOR™), vatanidipine, clevidipine, lercanidipine, or dilitiazem.

In some embodiments, atrasentan, or a pharmaceutically acceptable salt thereof, and a SGLT-2 inhibitor may be co-administered with one or more renin inhibitors such as aliskiren (such as TEKTURNA™).

In some embodiments, atrasentan, or a pharmaceutically acceptable salt thereof, and a SGLT-2 inhibitor may be co-administered with one or more aldosterone receptor antagonists such as eplerenone (such as INSPRA™) or spironolactone (such as ALDACTONE™).

In some embodiments, atrasentan, or a pharmaceutically acceptable salt thereof, and a SGLT-2 inhibitor may be co-administered with one or more alpha blockers such as dozazosin (such as CARDURA™) phenoxybenzamine (such as DIBENZYLINE™) terazosin (such as HYTRIN™), CDR1-93/478, or CR-2991.

In some embodiments, atrasentan, or a pharmaceutically acceptable salt thereof, and a SGLT-2 inhibitor may be co-administered with one or more beta blockers such as timolol (such as BLOCARDEN™) carteolol (such as CARTROL™), carvedilol (such as COREG™), nadolol (such as CORGARD™), propranolol (such as INNOPRAN XL™) betaxolol (such as KERLONE™) penbutolol (such as LEVATOL™), metoprolol (such as LOPRESSOR™ or TOPROL-XL™), atenolol (such as TENORMIN™), pindolol (such as VISKEN™), or bisoprolol.

In some embodiments, atrasentan, or a pharmaceutically acceptable salt thereof, and a SGLT-2 inhibitor may be co-administered with one or more alpha-beta blockers such as labetalol (such as NORMODYNE™ or TRANDATE™).

In some embodiments, atrasentan, or a pharmaceutically acceptable salt thereof, and a SGLT-2 inhibitor may be co-administered with one or more central antiadrenergics such as methyldopa (such as ALDOMET™), clonidine (such as CATAPRES™ or CATAPRES-TTS™), guanfacine (such as TENEX™), or guanabenz (such as WYTENSIN™).

In some embodiments, atrasentan, or a pharmaceutically acceptable salt thereof, and a SGLT-2 inhibitor may be co-administered with one or more glycosides/inotropic agents such as digoxin (such as LANOXIN™).

In some embodiments, atrasentan, or a pharmaceutically acceptable salt thereof, and a SGLT-2 inhibitor may be co-administered with one or more alpha glucosidase inhibitors, such as miglitol (such as GLYSET™) or acarbose (such as PRECOSE™).

In some embodiments, atrasentan, or a pharmaceutically acceptable salt thereof, and a SGLT-2 inhibitor may be co-administered with one or more biguanides, such as roseiglitazone (such as AVANDAMET™) or metformin (such as GLUCOPHAGE™ or GLUCOPHAGE XR™).

In some embodiments, atrasentan, or a pharmaceutically acceptable salt thereof, and a SGLT-2 inhibitor may be co-administered with one or more insulins, such as HUMALOG™, HUMALOG 50/50™, HUMALOG 75/25™, HUMULIN 50/50™ HUMALIN 75/25™, HUMALIN L™, HUMALIN N™, HUMALIN®, HUMALIN R U-500™, HUMALIN U™, ILETIN II LENTE™, ILETIN II NPH™, ILETIN II REGULAR™ LANTUS™, NOVOLIN 70/30™, NOVILIN N™, NOVILIN R™, NOVOLOG™, or VELOSULIN BR™, and EXUBERA™.

In some embodiments, atrasentan, or a pharmaceutically acceptable salt thereof, and a SGLT-2 inhibitor may be co-administered with one or more meglitnides, such as repaglinide (such as PRANDIN™) or nateglinide (such as STARLIX™).

In some embodiments, atrasentan, or a pharmaceutically acceptable salt thereof, and a SGLT-2 inhibitor may be co-administered with one or more sulfonylureas, such as glimepiride (such as AMARYL™), glyburide (such as DIABETA™, GLYNASE PRESTAB™ or MICRONASE™), or glipizide (such as GLUCOTROL™, or GLUCOTROL XL™).

In some embodiments, atrasentan, or a pharmaceutically acceptable salt thereof, and a SGLT-2 inhibitor may be co-administered with one or more thiazolidinediones, such as pioglitazone (such as ACTOS™) or rosiglitazone (such as AVANDIA™).

In some embodiments, atrasentan, or a pharmaceutically acceptable salt thereof, and a SGLT-2 inhibitor may be co-administered with niacin or one or more nicotinic acid derivatives, such as NIACOR™, NIASPAN™, NICOLAR™, or SLO-NIACIN™.

In some embodiments, atrasentan, or a pharmaceutically acceptable salt thereof, may be co-administered with one or more fabric acid derivatives, such as clofibrate (such as ATROMID-S™), gemfibrozil (such as LOPID™), or fenofibrate (such as TRICOR™).

In some embodiments, atrasentan, or a pharmaceutically acceptable salt thereof, and a SGLT-2 inhibitor may be co-administered with one or more bile acid sequestrants, such as colestipol (such as COLESTID™), cholestyramine (such as LOCHOLEST™, PREVALITE™, QUESTRAN™, or QUESTRAN LIGHT™), or colesevelam (such as WELCHOL™).

In some embodiments, atrasentan, or a pharmaceutically acceptable salt thereof, and a SGLT-2 inhibitor may be co-administered with one or more cholesterol absorption inhibitors, such as ezetimibe (such as ZETIA™).

In some embodiments, atrasentan, or a pharmaceutically acceptable salt thereof, and a SGLT-2 inhibitor may be co-administered with one or more 3-hydroxy-3-methylglutaryl coenzyme A (HMG-CoA) reductase inhibitors (statins) such as fluvastatin (such as LESCOL™), atorvastatin (such as LIPITOR™), lovastatin (such as ALTOCOR™ or MEVACOR™), pravastatin (such as PRAVACHOL™), rosuvastatin (such as CRESTOR™), simvastatin (such as ZOCOR™), or pitavastatin.

In some embodiments, atrasentan, or a pharmaceutically acceptable salt thereof, and a SGLT-2 inhibitor may be co-administered with one or more additional agents. In some embodiments, the one or more additional agents is an immunosuppressant. In some embodiments, the one or more additional agents are selected from the group consisting of aminopterin, azathioprine, cyclosporin A, D-penicillamine, gold salts, hydroxychloroquine, leflunomide, methotrexate, minocycline, rapamycin, sulfasalazine, tacrolimus (FK506), and pharmaceutically acceptable salts thereof. As a non-limiting example, the one or more additional agents can be hydroxychloroquine.

In some embodiments, atrasentan, or a pharmaceutically acceptable salt thereof, and a SGLT-2 inhibitor may be co-administered with one or more additional therapeutic agents selected group the group consisting of GR-immunosuppressant (such as budenoside), MASP-2 antibodies (such as OMS721), dual ET1 Å/ARB inhibitors (such as sparsentan), B cell modulators (e.g., APRIL modulators such as atacicept, APL-2, and VIS649), SYK inhibitor (such as fosamatinib), complement factor 3 convertase inhibitor (such as LNP023), NRF2 activator (such as Bardoxolone), and RNAi therapeutic targeting the C5 component of the complement pathway (e.g., cemdisiram).

In some embodiments, the present disclosure relates to the use of atrasentan, or a pharmaceutically acceptable salt thereof, and a SGLT-2 inhibitor in combination with a third therapeutic for treating a condition as described in the various embodiments of the disclosure.

In some embodiments, the present disclosure relates to the use of atrasentan, or a pharmaceutically acceptable salt thereof, and a SGLT-2 inhibitor for treating a condition as described in the various embodiments of the disclosure, wherein the use comprises one or more additional therapeutic agents.

In some embodiments, the present disclosure relates to a pharmaceutical composition comprising atrasentan, or a pharmaceutically acceptable salt thereof, and a SGLT-2 inhibitor and further comprising one or more additional therapeutic agent. In some embodiments, the present disclosure relates to a pharmaceutical composition comprising atrasentan, or a pharmaceutically acceptable salt thereof, and a pharmaceutical composition comprising a SGLT-2 inhibitor and further comprising one or more a pharmaceutical compositions comprising additional therapeutic agent(s).

In some embodiments, the one or more additional therapeutic agent inhibits one or more elements of the renin-angiotensin-aldosterone system. In some embodiments, the one or more additional therapeutic agent is selected from the group consisting of diuretics, angiotensin converting enzyme (ACE) inhibitors, and angiotensin II receptor (ARB) blockers. In certain particular embodiments, the one or more additional therapeutic agent is selected from the group consisting of angiotensin converting enzyme (ACE) inhibitors and angiotensin II receptor blockers (ARBs). In certain embodiments, the one or more additional therapeutic agent is selected from one or more angiotensin converting enzyme inhibitors. In certain embodiments, the one or more additional therapeutic agent is selected from one or more angiotensin II receptor blockers. In certain embodiments, the one or more additional therapeutic agents comprises one or more ACE inhibitors, one or more ARBs and one or more diuretics. For example, the one or more inhibitors of the renin-angiotensin system can be ACE inhibitor, ARB, or a combination thereof. For example, the ACE inhibitor can be selected from: quinapril, fosinopril perindopril, captopril, enalapril, enalaprilat, ramipril, cilazapril, delapril, fosenopril, zofenopril, indolapril, benazepril, lisinopril, spirapril, trandolapril, perindep, pentopril, moexipril, rescinnamine, and pivopril. For example, the ARB can be selected from: candesartan, candesartan cilexetil, eprosartan, irbesartan, losartan, olmesartan, olmesartan medoxomil, telmisartan, valsartan, azilsartan medoxomil, and BRA-657.

In some embodiments, atrasentan, or a pharmaceutically acceptable salt thereof, may be co-administered with a SGLT-2 inhibitor and one or more ACE inhibitors and/or one or more ARBs. In some embodiments, atrasentan, or a pharmaceutically acceptable salt thereof, may be co-administered with a SGLT-2 inhibitor and one or more ACE inhibitors. In some embodiments, atrasentan, or a pharmaceutically acceptable salt thereof, may be co-administered with a SGLT-2 inhibitor and one or more ARBs. In some embodiments, atrasentan, or a pharmaceutically acceptable salt thereof, may be co-administered with a SGLT-2 inhibitor, an ACE inhibitor, and an ARB. In some embodiments, atrasentan, or a pharmaceutically acceptable salt thereof, may be co-administered with a SGLT-2 inhibitor, an ACE inhibitor or an ARB and a diuretic.

In some embodiments, atrasentan, or a pharmaceutically acceptable salt thereof, may be co-administered with a SGLT-2 inhibitor may be administered with one or more calcium channel blockers, renin inhibitors, and aldosterone antagonists.

It is understood that the examples and embodiments described herein are for illustrative purposes only and that various modifications or changes in light thereof will be suggested to persons skilled in the art and are to be included within the spirit and purview of this application and scope of the appended claims. All publications, patents, patent applications, and sequence accession numbers cited herein are hereby incorporated by reference in their entirety for all purposes.

The disclosure will be more fully understood by reference to the following examples. They should not, however, be construed as limiting the scope of the disclosure. It is understood that the examples and embodiments described herein are for illustrative purposes only and that various modifications or changes in light thereof will be suggested to persons skilled in the art and are to be included within the spirit and purview of this application and scope of the appended claims.

### EXAMPLES

### Example 1. SONAR Study

A double-blind, randomized, placebo-controlled trial of DKD patients was conducted at 689 sites in 41 countries. The trial was conducted in two phases, an enrichment period and a double-blind treatment period. For the enrichment period, eligible patients were 18-85 years old with type 2 diabetes, an estimated glomerular filtration rate (eGFR) of 25-75 mL/min per 1.73 m² of body surface area, a urine albumin-to-creatinine ratio (UACR) of 300-5000 mg/g, a serum albumin of at least 25 g/L, a brain natriuretic peptide (BNP) concentration (level) of no more than 200 pg/mL, a serum potassium of at least 3 to 5 mmol/L, and systolic blood pressure of 110-180 mm Hg. The patients were required to receive a stable, recommended or maximally tolerated dose of an ACE inhibitor or ARB for at least four weeks prior to beginning the enrichment period. Patients also received a diuretic prior to beginning the enrichment period. Exclusion criteria included: a diagnosis of or previous hospital admission for heart failure, a history of severe peripheral or facial edema, diagnosis of type 1 diabetes, history of pulmonary hypertension, pulmonary fibrosis, or any lung diseases requiring oxygen therapy, and known non-diabetic kidney disease. See Heerspink et al., Lancet 393:1937-1947 (2019).

During the six week enrichment period, patients received atrasentan hydrochloride 0.75 mg orally daily in addition to their then current treatment with an ACE inhibitor or an ARB and a diuretic. Responding patients (responders) with at least a 30% reduction in UACR, who did not have substantial fluid retention (defined as an increase in bodyweight of 3 kg or more and a BNP increase to 300 pg/mL or more), and who did not have an increase in serum creatinine of more than 0.5 mg/dL, and more than 20% from baseline, were eligible to proceed to the treatment period.

During the treatment period, two types of patients were studied. Responders were randomly assigned to receive either atrasentan 0.75 mg orally daily or a placebo. All patients and investigators were masked to treatment assignment. Patients continued to receive an ACE inhibitor or an ARB, and a diuretic during the treatment period. The primary endpoint for the responders was a composite of doubling of serum creatinine (sustained for >30 days) or ESRD (eGFR<15 mL/min per 1.73 m² sustained for >90 days, chronic dialysis for >90 days, kidney transplantation, or death from kidney failure) in the intent-to-treat population of all responders. Safety was assessed in all patients who received at least one dose of their assigned study treatment.

A second group of non-responding patients (those who had a UACR reduction of <30% during the enrichment period and no evidence of significant fluid retention) were also randomly assigned to atrasentan or placebo to establish whether renal benefit was observed in this population.

Patient summary: 11,087 patients were screened; 5,117 entered the enrichment period, and 4,711 completed the enrichment period. Of these, 2,648 patients were responders and were randomly assigned to the atrasentan group (n=1,325) or placebo group (n=1323). Median follow-up was 2.2 years (IQR 1.4-2.9). 79 (6.0%) of 1,325 patients in the atrasentan group. 105 (7.9%) of 323 in the placebo group had a primary composite renal endpoint event (hazard ratio [HR] 065 [95% CI 0.49-0.88]; p=0.0047). Fluid retention and anemia adverse events, which have been previously attributed to endothelin receptor antagonists, were more frequent in the atrasentan group than in the placebo group. Hospital admission for heart failure occurred in 47 (3.5%) of 1,325 patients in the atrasentan group and 34 (2.6%) of 1,323 patients in the placebo group (HR 1.33 [95% CI 0.85-2.07]; p=0.208). 58 (4.4%) patients in the atrasentan group and 52 (3.9%) in the placebo group died (HR 1.09 [95% CI 0.75-1.59]; p=0.65).

Atrasentan reduced the risk of renal events in patients with diabetes and chronic kidney disease who were selected to optimize efficacy and safety. The data support a potential role for selective endothelin receptor antagonists in protecting renal function in patients with type 2 diabetes at high risk of developing end-stage kidney disease.

### Example 2. Concomitant Medication Use of Patients in the SONAR Study

An initial analysis of the concomitant medication use by patients during the enrichment period suggested that the albuminuria lowering effect of atrasentan in the responding patients was similar whether or not SGLT-2 inhibitors were taken by the patients. See Heerspink et al., Diabetes Obes Metab., 20:1829-1835 (2018). 42 responder patients and 9 patients in the non-responder group had used a SGLT-2 inhibitor at the start of enrichment. For responders, the change in UACR from baseline was −48.8% (−47.8 to −49.7) without concomitant SGLT-2 inhibitor use and −53.2% (−56.7 to −47.8) with concomitant SGLT-2 inhibitor use. For non-responders, the change in UACR from baseline was −1.3% (−6.5 to +3.9) without concomitant SGLT-2 inhibitor use and +10.4% (−29.1 to +50.0) with concomitant SGLT-2 inhibitor use. It was concluded that the UACR reduction was consistent in both groups.

This data also revealed a significant, unexpected effect of the SGLT-2 inhibitor (SGLT2i) co-administration. Referring to the following Table 1, case-control matched cohort data from the study is shown.

TABLE 1
Case-control Matched cohort

| | Atrasentan and | | Atrasentan | |
|---|---|---|---|---|
| | SGLT2i (N = 14) | | (N = 42) | |
| Age, years | 66.1 | (6) | 62.7 | (8) |
| Female sex, n/% | 4 | (28.6) | 10 | (23.8) |
| Race | | | | |
| White | 9 | (64.3) | 30 | (71.4) |
| Black | 1 | (7.1) | 5 | (11.9) |
| Asian | 4 | (28.6) | 7 | (16.7) |
| Hispanic | 0 | (0) | 0 | (0) |
| Other | 0 | (0) | 0 | (0) |
| Body weight, kg | 101.6 | (27) | 100.1 | (25) |
| Blood Pressure, mmHg | | | | |
| Systolic | 142.4 | (23) | 142.8 | (20) |
| Diastolic | 73.4 | (8) | 80.2 | (12) |
| Hba1c, % | | | | |
| Hemoglobin, g/dL | 13.4 | (1.8) | 13.1 | (1.7) |
| eGFR, ml/min/1.73 m² | 42.3 | (8) | 41.7 | (13) |
| Urinary albumin:creatinine ratio, | 465 | [353-873] | 491 | [319-701] |
| mg/g | | | | |
| B-type Natriuretic Peptide, pg/ml | 52 | [26-93] | 44 | [27-81] |

Referring to the following Table 2, a comparison of parameters in patients concomitantly treated with atrasentan hydrochloride and a SGLT-2 inhibitor (SGLT2i) versus patients treated with atrasentan alone. The data indicated that concomitant treatment with atrasentan and a SGLT-2 inhibitor significantly reduced fluid retention, B-type natriuretic peptide levels, UACR, and eGFR, as compared to treatment with atrasentan, but not a SGLT-2 inhibitor.

TABLE 2
Changes in renal efficacy parameters and proxies for fluid retention

| | Atrasentan and | | Atrasentan | |
|---|---|---|---|---|
| | SGLT2i (N = 14) | | (N = 42) | |
| Fluid retention | | | | |
| Body weight, kg | −0.7 | (2.0) | 1.1 | (1.8) |
| Hematocrit, % | −2.1 | (2.0) | −2.5 | (2.2) |
| Hemoglobin, g/dL | −0.7 | (9.3) | −0.9 | (0.7) |
| B-type Natriuretic Peptide, % | −4.1 | (−35.8 to 43.1) | 6.9 | (−13.8 to 32.7) |
| Renal efficacy | | | | |
| Systolic Blood Pressure, mmHg | −13.3 | (16) | −9.2 | (16) |
| Urinary albumin:creatinine ratio, % | −54.7 | (−62.8 to −44.9) | −38.0 | (−47.8 to −26.4) |
| eGFR, ml/min/1.73 m² | −3.5 | (5.2) | −0.6 | (7.1) |

Referring to Table 3, further analysis of the data from the enrichment period revealed that prior and concomitant administration of a SGLT-2 inhibitor with atrasentan resulted in the largest reduction in UACR.

TABLE 3
Effect of Concomitant SGLT2 Inhibitor Use on UACR Change
by Study Period and Treatment Log Transformed UACR

| Enrichment | | UACR | | |
|---|---|---|---|---|
| Atrasentan | Statistic | Baseline | Final | Change |
| Prior | N | 6 | 6 | 6 |
| SGLT2 only | Mean (SD) | 1554.98 (1361.425) | 1457.19 (1448.751) | −97.80 (194.266) |
| | Min, Max | 142.0, 3928.0 | 83.0, 3862.0 | −284.0, 242.0 |
| | Median | 1149.44 | 939.56 | −101.00 |
| Concomitant | N | 64 | 64 | 64 |
| sGLT2 only | Mean (SD) | 1279.39 (1324.067) | 809.47 (956.422) | −469.92 (680.739) |
| | Min, Max | 129.0, 7000.0 | 100.0, 4781.0 | −3786.0, 761.0 |
| | Median | 737.50 | 437.50 | −311.00 |
| Prior and | N | 5 | 5 | 5 |
| concomitant | Mean (SD) | 489.08 (269.276) | 379.37 (556.751) | −109.71 (366.699) |
| sGLT2 | Min, Max | 136.0, 876.0 | 92.0, 1374.0 | −421.0, 498.0 |
| | Median | 492.40 | 136.00 | −261.00 |
| No prior or | N | 4768 | 4768 | 4768 |
| concomitant | Mean (SD) | 1256.95 (1153.800) | 891.95 (1352.518) | −365.00 (1213.365) |
| SGLT2 | Min, Max | 3.0, 11414.0 | 4.0, 65669.0 | −9393.0, 63666.0 |
| | Median | 876.00 | 540.16 | −257.00 |

### Example 3. Concomitant SGLT-2 Inhibitor Use of Patients in the SONAR Study

Further analysis of concomitant SGLT-2 inhibitor use by patients during the enrichment period suggested that initiation of treatment with atrasentan and a SGLT-2 inhibitor provide a syngergistic reduction in albuminuria while offsetting fluid retention.

During the enrichment period, 14 subjects with type 2 diabetes and DKD started treatment with a SGLT2i in combination with atrasentan. SGLT-2 inhibitors included canagliflozin (N=6), empagliflozin (N=4), dapagliflozin (N=3), and luseogliflozin (N=1). These 14 subjects were matched in a 1 to 3 ratio with patients who used atrasentan alone, and the baseline characteristics between the combination group and atrasentan alone group were balanced. See Table 4.

TABLE 4
Combination Matched Cohort

| | Atrasentan and | | Atrasentan | | Standardized | Atrasentan | |
|---|---|---|---|---|---|---|---|
| | SGLT2 (N = 14) | | (N = 42) | | Difference | (N = 5093) | |
| Age, years, mean (SD) | 66.1 | (6) | 65.0 | (10) | 0.131 | 64.4 | (8.8) |
| Female sex, n/% | 4 | (28.6) | 13 | (31.0) | 0.051 | 1390 | (27.2) |
| Race, n % | | | | | 0.121 | | |
| White | 9 | (64.3) | 25 | (69.0) | | 3001 | (58.9) |
| Black | 1 | (7.1) | 5 | (11.9) | | 351 | (6.9) |
| Asian | 4 | (28.6) | 9 | (21.4) | | 1552 | (30.5) |
| Other | 0 | (0) | 3 | (7.1) | | 189 | (3.7) |
| Body weight, kg mean | 101.6 | (27) | 101.7 | (27) | 0.003 | 86.0 | (20) |
| (SD) | | | | | | | |
| Systolic Blood Pressure, | 142.4 | (23) | 142.7 | (14) | 0.015 | 137.5 | (15) |
| mmHg mean (SD) | | | | | | | |
| Hba1c, % mean (SD) | 8.1 | (1.4) | 7.9 | (1.4) | 0.122 | 7.6 | (1.5) |
| eGFR, mL/min/1.73 m² | 42.3 | (8) | 40.6 | (13) | 0.155 | 41.5 | (13) |
| mean (SD) | | | | | | | |
| Urinary | 465 | [353-873] | 632 | [414-1111] | 0.070 | 871 | [474-1675] |
| albumin:creatinine ratio, | | | | | | | |
| mg/g median [IQR] | | | | | | | |
| B-type Natriuretic Peptide, | 52 | [26-93] | 51 | [28-86] | 0.089 | 50 | [27-91] |
| pg/ml median [IQR] | | | | | | | |
| Diuretics, n/% | 13 | (92.9) | 38 | (90.5) | 0.084 | 4112 | (80.7) |

We examined the effect of combined treatment with atrasentan and SGLT2i versus atrasentan alone on body weight and UACR as surrogates for fluid retention and kidney protection, respectively during the enrichment period of the SONAR trial. Change in body weight was used as a surrogate for fluid retention. See, e.g., Hoekman, et al., Clin. J. Am. Soc. Nephrol. 2013; 9(3):490-498. The change in BNP was monitored as an additional surrogate for fluid retention (each 1 kg increase in body weight during the enrichment period was associated with a 7.3% increase in BNP (p-value<0.001)).

In subjects treated with atrasentan alone, body weight increased by 0.6 kg after six weeks (95% CI 0.0 to 1.1). In contrast, body weight decreased by 0.7 kg in subjects administered atrasentan and a SGLT-2 inhibitor (95% CI −0.3 to 1.6), which provided a between-group difference of 1.2 kg (95% CI 0.1 to 2.3, p=0.028). Similarly, the combination treatment group also saw a reduction in BNP levels relative to the atrasentan alone group See Table 5.

TABLE 5
Cardio-renal Risk Markers in Combination Matched Cohort

| | Atrasentan and | | Atrasentan | |
|---|---|---|---|---|
| | SGLT2i (N = 14) | | (N = 42) | |
| Urinary albumin:creatinine ratio, % | −54.7 | (−64.6 to −42.0) | −37.6 | (−45.9 to −28.0) |
| Systolic Blood Pressure, mmHg | −13.3 | (−21.4 to −5.3) | −6.7 | (−10.1 to −3.3) |
| eGFR, ml/min/1.73 m² | −3.5 | (−6.4 to −0.6) | −2.1 | (−3.7 to −0.4) |
| Body weight, kg | −0.7 | (−1.6 to 0.3) | 0.6 | (0.0 to 1.1) |
| B-type Natriuretic Peptide, % | −4.1 | (−35.3 to 42.1) | 5.1 | (−16.5 to 32.3) |

*Geometric mean (95% confidence interval)
95% confidence intervals based on marginal means from ANCOVA model.

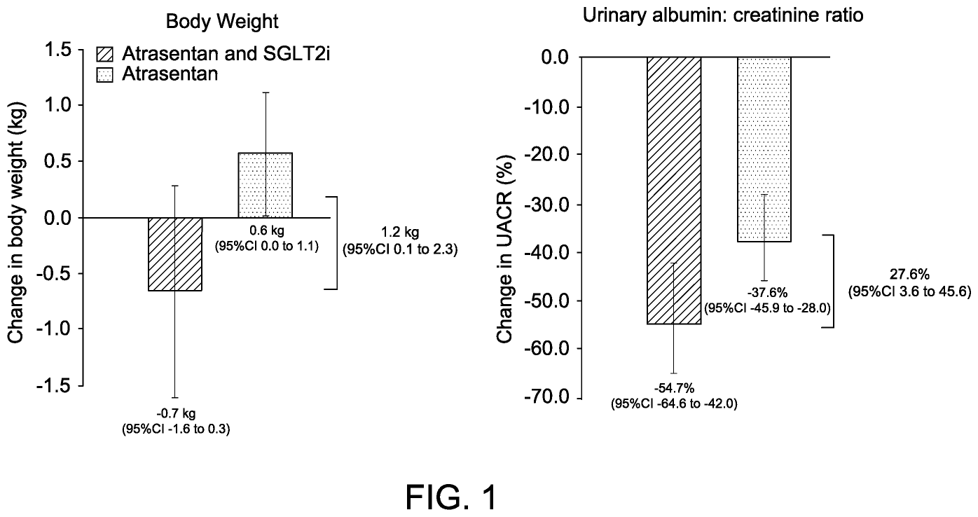

The combination treatment was also associated with a 27.6% (95% CI 3.6% to 45.6%, p=0.028) greater reduction in UACR compared to atrasentan alone. See FIG. 1. Analysis of the combination group relative to all subjects treated with atrasentan alone during the enrichment period provided similar results. See Table 6. This analysis demonstrates that administration of a SGLT-2 inhibitor with atrasentan not only reduces fluid retention, but surprisingly provides improved UACR and kidney protection in subjects with DKD relative to treatment with atrasentan alone.

TABLE 6
Cardio-renal Risk Markers in the Enrichment Period

| | Atrasentan and | | Atrasentan | |
|---|---|---|---|---|
| | SGLT2i (N = 14) | | (N = 5093) | |
| Urinary albumin:creatinine ratio, %* | −54.7 | (−64.9 to −41.6) | −37.6 | (−38.4 to −36.8) |
| Systolic Blood Pressure, mmHg | −13.3 | (−20.3 to −6.3) | −4.7 | (−5.0 to to −4.3) |
| eGFR, ml/min/1.73 m² | −3.5 | (−7.1 to 0.1) | −1.0 | (−1.2 to −0.9) |
| Body weight, kg | −0.7 | (−1.5 to 0.2) | 0.6 | (0.5 to 0.6) |
| B-type Natriuretic Peptide, %* | −4.1 | (−35.8 to 43.1) | 8.3 | (6.4 to 10.3) |

*Geometric mean (95% confidence interval)
95% confidence intervals based on marginal means from ANCOVA model.

1. A method of treating diabetic kidney disease, or chronic kidney disease associated with diabetes, comprising administering a therapeutically effective amount of atrasentan, or a pharmaceutically acceptable salt thereof, and a therapeutically effective amount of a SGLT-2 inhibitor to a subject in need thereof.

2. The method of claim 1, wherein said administration delays progressive renal function decline in the subject.

3. (canceled)

4. The method of claim 1, wherein treatment outcome in the subject is improved relative to treatment outcome in a subject not administered atrasentan, or a pharmaceutically acceptable salt thereof, and the SGLT-2 inhibitor.

5. The method of claim 1, wherein the urine albumin to creatinine ratio (UACR) of the subject is reduced, wherein the UACR of the subject following administration of the atrasentan, or a pharmaceutically acceptable salt thereof, and the SGLT-2 inhibitor is less than the UACR of the subject prior to the first administration of atrasentan, or a pharmaceutically acceptable salt thereof, and the SGLT-2 inhibitor.

6. The method of claim 1, wherein fluid retention in the subject is reduced, wherein fluid retention after administration of the SGLT-2 inhibitor is less than fluid retention prior to the administration of the SGLT-2 inhibitor.

7. (canceled)

8. The method of claim 1, wherein B-type natriuretic peptide (BNP) levels in the subject is reduced, and wherein the BNP level of the subject following administration of the atrasentan, or a pharmaceutically acceptable salt thereof, and the SGLT-2 inhibitor is less than the BNP level of the subject upon administration of atrasentan, or a pharmaceutically acceptable salt thereof, or the SGLT-2 inhibitor alone.

9. The method of claim 1, wherein estimated glomerular filtration rate (eGFR) of the subject is stabilized, and wherein the eGFR of the subject following administration of the atrasentan, or a pharmaceutically acceptable salt thereof, and the SGLT-2 inhibitor is stabilized relative to a subject not administered the atrasentan, or a pharmaceutically acceptable salt thereof, and the SGLT-2 inhibitor.

10. The method of claim 1, wherein the subject's UACR, body weight or fluid retention, BNP level, rate of decrease of eGFR, or a combination of any of the foregoing are reduced following administration of the atrasentan, or a pharmaceutically acceptable salt thereof, and the SGLT-2 inhibitor, wherein the reduction is greater relative to a subject not administered atrasentan, or a pharmaceutically acceptable salt thereof, and the SGLT-2 inhibitor.

11. The method of claim 10, wherein at least two of the subject's UACR, body weight or fluid retention, BNP level, and rate of decrease of eGFR, are reduced.

12. The method of claim 10, wherein at least three of the subject's UACR, body weight or fluid retention, BNP level, and rate of decrease of eGFR, are reduced.

13. The method of claim 10, wherein the subject's UACR, body weight or fluid retention, BNP level, and rate of decrease of eGFR are reduced.

14. The method of claim 1, wherein the subject has Type 2 diabetes.

15. The method of claim 1, wherein the subject has Type 1 diabetes.

16. The method of claim 1, further comprising administering an Angiotensin-Converting Enzyme (ACE) inhibitor, an Angiotensin II Receptor Blocker (ARB), a diuretic, or a combination thereof to the subject.

17. The method of claim 16, wherein an ACE inhibitor and a diuretic are administered to the subject.

18. The method of claim 16, wherein an ARB and a diuretic are administered to the subject.

19. The method of claim 16, wherein an ACE inhibitor and an ARB are administered to the subject.

20. The method of claim 16, wherein an ACE inhibitor, an ARB, and a diuretic are administered to the subject.

21. The method of claim 18, wherein the subject has been administered a maximally tolerated stable dose of an ACE inhibitor or an ARB for at least 4 weeks prior to the first administration of atrasentan, or a pharmaceutically acceptable salt thereof.

22. The method of claim 18, wherein the subject has been administered a maximally tolerated stable dose of an ACE inhibitor or an ARB for at least 10 weeks prior to the first administration of atrasentan, or a pharmaceutically acceptable salt thereof.

23. The method of claim 18, wherein the subject has been administered a maximally tolerated stable dose of an ACE inhibitor or an ARB for at least 12 weeks prior to the first administration of atrasentan, or a pharmaceutically acceptable salt thereof.

24. The method of claim 21, wherein the subject has also been administered a diuretic prior to the first administration of atrasentan, or a pharmaceutically acceptable salt thereof.

25. The method of claim 18, wherein the subject is administered a maximally tolerated stable dose of an ACE inhibitor or an ARB.

26. The method of claim 24, wherein the diuretic is selected from the group consisting of: hydrochlorothiazide, trichlormethiazide, hydroflumethiazide, quinethazone, metolazone, chlorothiazide, chlorthalidone, indapamide, methyclothiazide bemetanide, torsemide, piretanide, ethacrynic acid, bumetanide, furosemide, triamterene, spironolactone, eplerenone, and amiloride.

27. The method of claim 18, wherein the ACE inhibitor is selected from the group consisting of: quinapril, fosinopril perindopril, captopril, enalapril, enalaprilat, ramipril, cilazapril, delapril, fosenopril, zofenopril, indolapril, benazepril, lisinopril, spirapril, trandolapril, perindep, pentopril, moexipril, rescinnamine, and pivopril.

28. The method of claim 18, wherein the ARB is selected from the group consisting of: candesartan, candesartan cilexetil, eprosartan, irbesartan, losartan, olmesartan, olmesartan medoxomil, telmisartan, valsartan, azilsartan medoxomil, and BRA-657.

29. The method of claim 1, wherein the subject has been administered a SGLT-2 inhibitor for at least 12 weeks prior to the first administration of atrasentan, or a pharmaceutically acceptable salt thereof.

30. The method of claim 1, wherein the SGLT-2 inhibitor is selected from the group consisting of dapagliflozin, canagliflozin, ipragliflozin, empaglifozin, bexagliflozin, licogliflozin, janagliflozin (XZP-5695), tofogliflozin, ertugliflozin, henagliflozin (SHR-3824), enavogliflozin (DWP-16001), TA-1887 (3-(4-cyclopropylbenzyl)-4-fluoro-1-(β-D-glucopyranosyl)-1H-indole), indole-N-glycoside 18 (3-(4-ethylbenzyl)-1-(β-D-glucopyranosyl)-1H-indole), sotagliflozin, luseogliflozin, sergliflozin etabonate, remogliflozin, remogliflozin etabonate, and T-1095 (((2R,3S,4S,5R,6S)-6-(2-(3-(benzofuran-5-yl)propanoyl)-3-hydroxy-5-methylphenoxy)-3,4,5-trihydroxytetrahydro-2H-pyran-2-yl) etabonate).

31. The method of claim 1, wherein the SGLT-2 inhibitor is selected from the group consisting of bexagliflozin, canagliflozin, dapagliflozin, empagliflozin, ertugliflozin, ipragliflozin, luseogliflozin, remogliflozin, serfliflozin, licofliglozin, sotagliflozin, and tofogliflozin.

32. The method of claim 1, wherein the SGLT-2 inhibitor is canagliflozin, dapagliflozin, empagliflozin, or ertugliflozin.

33. The method of claim 1, wherein the atrasentan is administered as a pharmaceutically acceptable salt.

34. The method of claim 1, wherein the atrasentan is administered as atrasentan hydrochloride or atrasentan mandelate.

35. The method of claim 1, wherein the atrasentan is administered as atrasentan hydrochloride.

36. The method of claim 1, wherein the atrasentan is administered as a free base.

37. The method of claim 1, wherein the therapeutically effective amount of atrasentan, or a pharmaceutically acceptable salt thereof, is from about 0.20 mg to about 1.5 mg of atrasentan, or an equivalent amount of a pharmaceutically acceptable salt thereof.

38. The method of claim 1, wherein the therapeutically effective amount of atrasentan, or a pharmaceutically acceptable salt thereof, is from about 0.25 mg to about 1.25 mg of atrasentan, or an equivalent amount of a pharmaceutically acceptable salt thereof.

39. The method of claim 1, wherein the therapeutically effective amount of atrasentan, or a pharmaceutically acceptable salt thereof, is from about 0.40 mg to about 0.85 mg of atrasentan, or an equivalent amount of a pharmaceutically acceptable salt thereof.

40. The method of claim 1, wherein the therapeutically effective amount of atrasentan, or a pharmaceutically acceptable salt thereof, is about 0.75 mg of atrasentan, or an equivalent amount of a pharmaceutically acceptable salt thereof.

41. The method of claim 1, wherein the subject has a UACR of about 300 mg/g to about 5000 mg/g prior to the first administration of atrasentan, or a pharmaceutically acceptable salt thereof.

42. The method of claim 1, wherein the subject has a UACR of about 300 mg/g to about 2000 mg/g prior to the first administration of atrasentan, or a pharmaceutically acceptable salt thereof.

43. The method of claim 1, wherein the subject has a UACR of about 300 mg/g to about 1000 mg/g prior to the first administration of atrasentan, or a pharmaceutically acceptable salt thereof.

44. The method of claim 1, wherein the subject has a UACR of about 300 mg/g to about 500 mg/g prior to the first administration of atrasentan, or a pharmaceutically acceptable salt thereof.

45. The method of claim 1, wherein the subject has a brain natriuretic peptide (BNP) concentration of about 200 pg/mL or less prior to the first administration of atrasentan, or a pharmaceutically acceptable salt thereof.

46. The method of claim 1, wherein the subject has a brain natriuretic peptide (BNP) concentration of about 20 pg/mL to about 100 pg/mL, prior to the first administration of atrasentan, or a pharmaceutically acceptable salt thereof.

47. The method of claim 1, wherein the subject has a serum potassium level of at least about 3.5 mmol/L prior to the first administration of atrasentan, or a pharmaceutically acceptable salt thereof, and the SGLT-2 inhibitor.

48. The method of claim 1, wherein the subject has systolic blood pressure of about 110 mm Hg to about 180 mm Hg prior to the first administration of atrasentan, or a pharmaceutically acceptable salt thereof.

49. The method of claim 1, wherein the subject has been determined to have controlled serum glucose levels prior to the first administration of atrasentan, or a pharmaceutically acceptable salt thereof.

50. The method of claim 1, wherein the subject has an estimated glomerular filtration rate (eGFR) of about 75 mL/min/1.73 m² to about 25 mL/min/1.73 m² prior to the first administration of atrasentan, or a pharmaceutically acceptable salt thereof.

51. The method of claim 1, wherein the subject has a serum albumin level of at least 25 g/L prior to the first administration of atrasentan, or a pharmaceutically acceptable salt thereof.

52. The method of claim 1, wherein the subject maintains a potassium level within the normal physiologic range during treatment.

53. The method of claim 1, wherein the subject maintains a sodium level within the normal physiologic range during treatment.

54. The method of claim 1, wherein the fluid retention in the subject is reduced by at least about 1 kg after treatment with the atrasentan, or a pharmaceutically acceptable salt thereof, and the SGLT-2 inhibitor, for at least about 15 to about 30 days.

55. The method of claim 1, wherein the average rate of decrease in eGFR is reduced by from about 15% to about 30% after about 1 month to about 12 months of treatment with atrasentan, or a pharmaceutically acceptable salt thereof, and the SGLT-2 inhibitor.

56. The method of claim 1, wherein the UACR of the subject is reduced by from about 10% to about 30% after about 1 month to about 12 months of treatment with atrasentan, or a pharmaceutically acceptable salt thereof, and the SGLT-2 inhibitor.

57. The method of claim 1, wherein the BNP level of the subject is reduced by from about 1% to about 10% after about 1 month to about 12 months of treatment with atrasentan, or a pharmaceutically acceptable salt thereof, and the SGLT-2 inhibitor.

58. The method of claim 1, wherein the subject is concomitantly receiving a statin, a calcium channel blocker, a beta blocker, an aldosterone antagonist, fish oil, or a combination of any of the foregoing.

59. The method of claim 58, wherein the statin is selected from the group consisting of: atorvastatin, fluvastatin, lovastatin, pravastatin, rosuvastatin, simvastatin, and pitavastatin.

60. The method of claim 1, wherein the subject has not been previously diagnosed with one or more of IgA nephropathy, HIV/AIDS, HIV-related nephropathy or acute kidney failure.

61. The method of claim 1, wherein the subject is not currently being treated for one or more of IgA nephropathy, HIV/AIDS, HIV-related nephropathy or acute kidney failure.

62. The method of claim 1, wherein the subject is not currently diagnosed with cancer.

63. The method of claim 1, wherein the subject is not currently being treated for cancer.

64. The method of claim 62, wherein the cancer is lung cancer or prostate cancer.
